SEQUENCE LISTING (74 sequences)

SEQ ID NO: 1 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 2 - protein, 353 aa, Mus musculus
MDYTMEPNVTMTDYYPDFFTAPCDAEFLLRGSMLYLAILYCVLFVLGLLGNSLVILVLVGCKKLRSITDIYLLNLAASDLLFVLSIPFQTHNLLDQWVFGTAMCKVVSGLYYIGFFSSMFFITLMSVDRYLAIVHAVYAIKVRTASVGTALSLTVWLAAVTATIPLMVFYQVASEDGMLQCFQFYEEQSLRWKLFTHFEINALGLLLPFAILLFCYVRILQQLRGCLNHNRTRAIKLVLTVVIVSLLFWVPFNVALFLTSLHDLHILDGCATRQRLALAIHVTEVISFTHCCVNPVIYAFIGEKFKKHLMDVFQKSCSHIFLYLGRQMPVGALERQLSSNQRSSHSSTLDDIL

SEQ ID NO: 3 - protein, 356 aa, Macaca mulatta
MDYTLDPSMTTMTDYYYPDSLSSPCDGELIQRNDKLLLAVFYCLLFVFSLLGNSLVILVLVVCKKLRNITDIYLLNLALSDLLFVFSFPFQTYYQLDQWVFGTVMCKVVSGFYYIGFYSSMFFITLMSVDRYLAVVHAVYAIKVRTIRMGTTTLSLLVWLTAIMATIPLLVFYQVASEDGVLQCYSFYNQQTLKWKIFTNFEMNILGLLIPFTIFMFCYIKILHQLKRCQNHNKTKAIRLVLIVVIASLLFWVPFNVVLFLTSLHSMHILDGCSISQQLNYATHVTEIISFTHCCVNPVIYAFVGEKFKKHLSEIFQKSCSHIFIYLGRQMPRESCEKSSSCQQHSFRSSSIDYIL

SEQ ID NO: 4 - protein, 355 aa, Oryctolagus cuniculus
MDYTLEPNVTAVTDYYYPDIFSSPCDGELTQRRSKLPLAIFYCLLFVSGLLGNGLVILVLVAYKKLQSITDIYLLNLALSDLLFVFSFPFLTHYQLDQWVFGTPMCKLVSGLYYVGFFSNMFFITLMSVDRYLAVVHAVCALRVRTARTGVALSLAVWLTAIMATSPLLVFYQVVPEDGVLQCYLSYNQQTLRWKIFTHLEMNVLGLLIPFAVLMFCYVRILQQLRHCQNHNKTKAIKLVLIVVLASLLFWVPFNVVLFLTSLHNMHVLDGCVINQRLIYATHVTEAISFTHCCVNPVIYAFMGEKFKKYLSDIFQKSCSHVVFYIGRNVSRQGYERSSSFPPSSSRSSIMDHIL

SEQ ID NO: 5 - protein, 5 aa, synthetic construct
TFAMN

SEQ ID NO: 6 - protein, 17 aa, synthetic construct
RIRSKSNNYATYYADSV

SEQ ID NO: 7 - protein, 12 aa, synthetic construct
GKENNYGYAMDY

SEQ ID NO: 8 - protein, 16 aa, synthetic construct
RSSQSLLHSNGNTYLY

SEQ ID NO: 9 - protein, 7 aa, synthetic construct
RMSNLAS

SEQ ID NO: 10 - protein, 9 aa, synthetic construct
MQHLEYPFT

SEQ ID NO: 11 - protein, 123 aa, synthetic construct
EVQLVETGGGLVQPKGSLRLSCAASGFPFNTFAMNWVRQAPGKGLEWLARIRSKSNNYATYYADSVKDRFTISRDDSRRMLYLLMNNVKFEDTAIYYCVRGKENNYGYAMDYWGQGTSVTVSS

SEQ ID NO: 12 - protein, 112 aa, synthetic construct
DIVMTQAAPSVPVTPGESVSISCRSSQSLLHSNGNTYLYWFLQRPGQSPQLLIYRMSNLASGVPDRFSGSGSGTAFTLRISRVEAEDVGVYYCMQHLEYPFTFGIGTKLELK

SEQ ID NO: 13 - protein, 453 aa, synthetic construct
EVQLVETGGGLVQPKGSLRLSCAASGFPFNTFAMNWVRQAPGKGLEWLARIRSKSNNYATYYADSVKDRFTISRDDSRRMLYLLMNNVKFEDTAIYYCVRGKENNYGYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 219 aa, synthetic construct
DIVMTQAAPSVPVTPGESVSISCRSSQSLLHSNGNTYLYWFLQRPGQSPQLLIYRMSNLASGVPDRFSGSGSGTAFTLRISRVEAEDVGVYYCMQHLEYPFTFGIGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 15 aa, synthetic construct
CQQHSSRSSSVDYIL

SEQ ID NO: 16 - protein, 35 aa, synthetic construct
MDYTLDLSVTTVTDYYYPDIFSSPCDAELIQTNGK

SEQ ID NO: 17 - protein, 16 aa, synthetic construct
CSQIFNYLGRQMPRES

SEQ ID NO: 18 - protein, 355 aa, Homo sapiens
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 19 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 20 - protein, 355 aa, Homo sapiens
MTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 21 - protein, 360 aa, Homo sapiens
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 22 - protein, 352 aa, Homo sapiens [sequence omitted]

SEQ ID NO: 23 - protein, 374 aa, Homo sapiens
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 24 - protein, 378 aa, Homo sapiens
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 25 - protein, 369 aa, Homo sapiens
MTPTDFTSPIPNMADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 26 - protein, 9 aa, synthetic construct
MQHLEYPFT

SEQ ID NO: 27 - protein, 13 aa, synthetic construct
AASGFPFNTFAMN

SEQ ID NO: 28 - protein, 12 aa, synthetic construct
RIRSKSNNYATY

SEQ ID NO: 29 - protein, 14 aa, synthetic construct
VRGKENNYGYAMDY

SEQ ID NO: 30 - protein, 14 aa, synthetic construct
RSSQSLLHSNGNTY

SEQ ID NO: 31 - protein, 7 aa, synthetic construct
RMSNLAS

SEQ ID NO: 32 - protein, 9 aa, synthetic construct
MQHLEYPFT

SEQ ID NO: 33 - protein, 10 aa, synthetic construct
GFPFNTFAMN

SEQ ID NO: 34 - protein, 19 aa, synthetic construct
RIRSKSNNYATYYADSVKD

SEQ ID NO: 35 - protein, 12 aa, synthetic construct
GKENNYGYAMDY

SEQ ID NO: 36 - protein, 14 aa, synthetic construct
RSSQSLLHSNGNTY

SEQ ID NO: 37 - protein, 7 aa, synthetic construct
RMSNLAS

SEQ ID NO: 38 - protein, 9 aa, synthetic construct
MQHLEYPFT

SEQ ID NO: 39 - protein, 452 aa, synthetic construct
EVQLVETGGGLVQPKGSLRLSCAASGFPFNTFAMNWVRQAPGKGLEWLARIRSKSNNYATYYADSVKDRFTISRDDSRRMLYLLMNNVKFEDTAIYYCVRGKENNYGYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 40 - protein, 219 aa, synthetic construct
DIVMTQAAPSVPVTPGESVSISCRSSQSLLHSNGNTYLYWFLQRPGQSPQLLIYRMSNLASGVPDRFSGSGSGTAFTLRISRVEAEDVGVYYCMQHLEYPFTFGIGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 41 - protein, 449 aa, synthetic construct
EVQLVESGGGLVQPGGSLKLSCAASGFIFSNAVMYWVRQASGKGLEWVAIKTKFNNYATYYADAVKGRFTISRDDSKNMVYLQMNSLKTEDTAVYYCTAGDRNKPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 214 aa, synthetic construct
ETVVTQSPATLSLSPGERATLSCRASTSVITLLHWFQQKPGQAPRLLIHGASNLESRVPARFSGSGSGTDFTLTISSLEPEDFATYFCQQSWNDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 453 aa, synthetic construct
EVQLVESGGGLVQPGGSLKLSCAASGFTFNTYAMNWVRQASGKGLEWVARIRSKANNYATYYADSVKDRFTISRDDSKNTLYLQMNNLKTEDTAVYYCVRDRSRGEDYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 219 aa, synthetic construct
DIVMTQSPLSLPVTPGEPASISCRSSKSLLHSNANTYLYWFLQKPGQSPQLLIYRMSNLASGVPDRFSGSGSGTAFTLKISRVEAEDVGVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 452 aa, synthetic construct
EVQLLESGGGLVQPGGSLRLSCAASGFTFSSYGMHWVRQAPGKGLEWVSAINWNGGSTGYADSVKGRFTISRDNSKNTLYLQMNSLRAEDTAVYYCARGHHSGYDGRFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 200 aa, synthetic construct
QSVLTQPPSVSGAPGQRVTISCTGSSSNIGAGYNVHWYQQLPGTAPKLLIYTNNRRPSGVPDRFSGSKSGTSASLAISGLRSEDEADYYCAAWDASLSGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQV

SEQ ID NO: 47 - protein, 445 aa, synthetic construct
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 219 aa, synthetic construct
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 449 aa, synthetic construct
EVQLLESGGGLVQPGGSLRLSCAASGIDLSTYAMGWVRQAPGKGLEWVGLIHRSGRTYYATWAKGRFTISKDSSKNTLYLQMNSLRAEDTAVYYCTRSYPDYSATASIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 218 aa, synthetic construct
DIQVTQSPSSLSASVGDRVTITCQASENIANALAWYQQKPGKPPKFLIYGASNLASGVPSRFSGSGSGTDFTFTISSLQPEDIATYYCQQAYYGNSFVEGTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 449 aa, synthetic construct; identical to SEQ ID NO: 49

SEQ ID NO: 52 - protein, 218 aa, synthetic construct; identical to SEQ ID NO: 50

SEQ ID NO: 53 - protein, 10 aa, synthetic construct
GFTFNTYAMN

SEQ ID NO: 54 - protein, 19 aa, synthetic construct
RIRSKSNNYATYYADSVKD

SEQ ID NO: 55 - protein, 12 aa, synthetic construct
VRGLLRYRFFDV

SEQ ID NO: 56 - protein, 16 aa, synthetic construct
RSSKSLLHSNGNTYLY

SEQ ID NO: 57 - protein, 7 aa, synthetic construct
RMSNLAS

SEQ ID NO: 58 - protein, 9 aa, synthetic construct
MQHLEYPFT

SEQ ID NO: 59 - protein, 121 aa, synthetic construct
EVQLVESGGGLVQPGGSLKLSCAASGFTFNTYAMNWVRQASGKGLEWVGRIRSKSNNYATYYADSVKDRFTISRDDSKNTAYLQMNSLKTEDTAVYYCVRGLLRYRFFDVWGQGTTVTVSS

SEQ ID NO: 60 - protein, 112 aa, synthetic construct
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHLEYPFTFGGGTKVEIK

SEQ ID NO: 61 - protein, 451 aa, synthetic construct
EVQLVESGGGLVQPGGSLKLSCAASGFTFNTYAMNWVRQASGKGLEWVGRIRSKSNNYATYYADSVKDRFTISRDDSKNTAYLQMNSLKTEDTAVYYCVRGLLRYRFFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 219 aa, synthetic construct
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHLEYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 10 aa, synthetic construct
GFPFNTFAMN

SEQ ID NO: 64 - protein, 8 aa, synthetic construct
RSKSNNYA

SEQ ID NO: 65 - protein, 12 aa, synthetic construct
GKENNYGYAMDY

SEQ ID NO: 66 - protein, 16 aa, synthetic construct
RSSQSLLHSNGNTYLY

SEQ ID NO: 67 - protein, 7 aa, synthetic construct
RMSNLAS

SEQ ID NO: 68 - protein, 9 aa, synthetic construct
MQHLEYPFT

SEQ ID NO: 69 - protein, 8 aa, synthetic construct
GFPFNTFA

SEQ ID NO: 70 - protein, 10 aa, synthetic construct
IRSKSNNYAT

SEQ ID NO: 71 - protein, 14 aa, synthetic construct
VRGKENNYGYAMDY

SEQ ID NO: 72 - protein, 16 aa, synthetic construct
RSSQSLLHSNGNTYLY

SEQ ID NO: 73 - protein, 7 aa, synthetic construct
RMSNLAS

SEQ ID NO: 74 - protein, 6 aa, synthetic construct
HHHHHH

(19) United States
(12) Patent Application Publication
(10) Pub. No.: US 2026/0056209 A1
(43) Pub. Date: Feb. 26, 2026

(54) ANTI-CCR8 ANTIBODIES AND USES THEREOF

(71) Applicant: Gilead Sciences, Inc., Foster City, CA (US)

(72) Inventors: Kelli L. Boyd, Madison, TN (US); Rosanna Win, Daly City, CA (US)

(21) Appl. No.: 19/236,227

(22) Filed: Jun. 12, 2025

Related U.S. Application Data
(60) Provisional application No. 63/660,379, filed on Jun. 14, 2024.

(51) Int. Cl. G01N 33/68; C07K 16/28; G01N 33/569; G01N 33/574

(52) U.S. Cl. CPC G01N 33/6863; C07K 16/2866; G01N 33/56972; G01N 33/57492; C07K 2317/34; C07K 2317/565; G01N 2333/7158

(57) ABSTRACT

Provided herein are various embodiments relating to anti-CCR8 binding agents, that bind to CCR8, and uses thereof. Some of the embodiments include antibodies, fragments thereof, variants thereof, derivatives thereof, and binding polypeptides targeting the same that bind CCR8. Such anti-CCR8 binding agents can be used in methods to treat, for example, cancer.

### CROSS-REFERENCE TO RELATED APPLICATIONS

This application claims the benefit of priority of U.S. Provisional Application No. 63/660,379, filed Jun. 14, 2024, the entire contents of which is incorporated herein by reference.

### SEQUENCE LISTING

The instant application contains a Sequence Listing, which has been submitted via Patent Center. The Sequence Listing titled 210196-315002_US_SL.xml, which was created on May 23, 2025, and is 75,413 bytes in size, is hereby incorporated by reference it its entirety.

### FIELD OF INVENTION

Antibodies that bind to C-C Motif Chemokine Receptor 8 (CCR8) are provided. Methods of detecting the level of CCR8 expression and/or CCR8 expressing cells in a sample using such antibodies are provided. Methods of treatment that include detecting CCR8 using such antibodies and/or administering such antibodies are also provided. Related methods for monitoring the level of CCR8 expression and/or CCR8 expressing cells that are useful in diagnostic and/or therapeutic settings, such as in the treatment of cancer with an anti-CCR8 antibody, are also provided.

### BACKGROUND

CCR8 belongs to the G protein-coupled receptor (GPCR) family. CCR8 is primarily expressed on tumor regulatory T (Treg) cells, a type of immunosuppressive cell found in the tumor microenvironment. Inhibition of CCR8 in the context of cancer treatment is an expanding therapeutic area. However, there remains a need for antibodies that bind CCR8 for treatment of cancer and other diseases and disorders. There also remains a need for antibodies that bind to CCR8 in a sample from a patient treated with a CCR8 antibody. The emergence of therapeutic CCR8 antibodies poses a new problem for diagnostic CCR8 antibodies that remains to be solved, i.e., the need for non-competing antibodies that can reliably and specifically detect CCR8 in the samples of patients that may contain potentially interfering therapeutic CCR8 antibodies. The compositions and methods disclosed herein satisfy this need and provide related advantages.

### SUMMARY

In one aspect, provided herein is a system of specifically binding CCR8 in a sample obtained from a subject, the system comprising components comprising an isolated antibody or an antigen-binding fragment thereof that binds to human C-C motif chemokine receptor (CCR8), wherein the antibody or fragment thereof comprises a heavy chain variable (VH) region comprising an HCDR1 comprising the amino acid sequence of SEQ ID NO: 5, an HCDR2 comprising the amino acid sequence of SEQ ID NO: 6, an HCDR3 comprising the amino acid sequence of SEQ ID NO: 7; and a light chain variable (VL) region comprising an LCDR1 comprising the amino acid sequence of SEQ ID NO: 8, an LCDR2 comprising the amino acid sequence of SEQ ID NO: 9, and an LCDR3 comprising the amino acid sequence of SEQ ID NO: 10.

In a second aspect, provided herein is a system of detecting CCR8 expressing cells in a sample obtained from a subject, the system comprising components comprising an isolated antibody or an antigen-binding fragment thereof that binds to human C-C motif chemokine receptor (CCR8), wherein the antibody or fragment thereof comprises a heavy chain variable (VH) region comprising an HCDR1 comprising the amino acid sequence of SEQ ID NO: 5, an HCDR2 comprising the amino acid sequence of SEQ ID NO: 6, an HCDR3 comprising the amino acid sequence of SEQ ID NO: 7; and a light chain variable (VL) region comprising an LCDR1 comprising the amino acid sequence of SEQ ID NO: 8, an LCDR2 comprising the amino acid sequence of SEQ ID NO: 9, and an LCDR3 comprising the amino acid sequence of SEQ ID NO: 10, optionally wherein the components further comprise a detection reagent or an amplification reagent.

In some embodiments of the first or second aspect, the detection of the CCR8 expressing cells in a sample obtained from a subject with cancer indicates the presence of a diseased microenvironment related to CCR8 expression. In some embodiments, the diseased microenvironment is a tumor microenvironment (TME), and wherein the CCR8 expressing cells comprise tumor infiltrating CCR8+ Treg cells. In some embodiments, (a) each component is suspended in a solution, or any combination of components is suspended in the same solution; (b) each component is housed in a container, or any combination of components is housed in a container; (c) each component is provided in a device, or any combination of components is provided in a device; or any combination of (a)-(c).

In a third aspect, provided herein is a device for detecting CCR8 expressing cells in a sample obtained from a subject, the device comprising an isolated antibody or an antigen-binding fragment thereof that binds to human C-C motif chemokine receptor (CCR8), wherein the antibody or fragment thereof comprises a heavy chain variable (VH) region comprising an HCDR1 comprising the amino acid sequence of SEQ ID NO: 5, an HCDR2 comprising the amino acid sequence of SEQ ID NO: 6, an HCDR3 comprising the amino acid sequence of SEQ ID NO: 7; and a light chain variable (VL) region comprising an LCDR1 comprising the amino acid sequence of SEQ ID NO: 8, an LCDR2 comprising the amino acid sequence of SEQ ID NO: 9, and an LCDR3 comprising the amino acid sequence of SEQ ID NO: 10; and wherein the antibody or fragment thereof is comprised in a reagent that is housed in a container.

In a fourth aspect, provided herein is a kit for detecting CCR8 expressing cells in a sample obtained from a subject, the kit comprising an isolated antibody or an antigen-binding fragment thereof that binds to human C-C motif chemokine receptor (CCR8), wherein the antibody or fragment thereof comprises a heavy chain variable (VH) region comprising an HCDR1 comprising the amino acid sequence of SEQ ID NO: 5, an HCDR2 comprising the amino acid sequence of SEQ ID NO: 6, an HCDR3 comprising the amino acid sequence of SEQ ID NO: 7; and a light chain variable (VL) region comprising an LCDR1 comprising the amino acid sequence of SEQ ID NO: 8, an LCDR2 comprising the amino acid sequence of SEQ ID NO: 9, and an LCDR3 comprising the amino acid sequence of SEQ ID NO: 10; and instructions for using the antibody or fragment thereof for detecting CCR8 expressing cells in the sample obtained from a subject.

In a fifth aspect, provided herein is a method of detecting CCR8 expressing cells in a sample obtained from a subject, comprising: (a) contacting a sample with an isolated antibody or an antigen-binding fragment thereof that binds to human C-C motif chemokine receptor (CCR8), wherein the antibody or fragment thereof comprises a heavy chain variable (VH) region comprising an HCDR1 comprising the amino acid sequence of SEQ ID NO: 5, an HCDR2 comprising the amino acid sequence of SEQ ID NO: 6, an HCDR3 comprising the amino acid sequence of SEQ ID NO: 7; and a light chain variable (VL) region comprising an LCDR1 comprising the amino acid sequence of SEQ ID NO: 8, an LCDR2 comprising the amino acid sequence of SEQ ID NO: 9, and an LCDR3 comprising the amino acid sequence of SEQ ID NO: 10; and (b) detecting the level of CCR8 expressing cells in said sample.

In a sixth aspect, provided herein is a method of diagnosing a diseased microenvironment associated with CCR8 expression in a subject, comprising: (a) obtaining a sample from the subject; (b) performing a method for detecting CCR8 expressing cells in a sample provided herein; and (c) associating the level of CCR8 expressing cells with a diseased microenvironment.

In a seventh aspect, provided herein is a method of selecting a subject suspected of having or having cancer for treatment with an anti-CCR8 agent, the method comprising: (a) obtaining a sample from the subject; (b) performing a method for detecting CCR8 expressing cells in a sample provided herein; (c) comparing the level of the subject's CCR8 expressing cells with a threshold level of CCR8 expressing cells; and (d) selecting the subject for treatment with an anti-CCR8 agent if the level of the subject's CCR8 expressing cells exceed the threshold level of CCR8 expressing cells.

In some embodiments, the CCR8 expressing cell is a CCR8+ Treg cell.

In an eight aspect, provided herein is a method of treating cancer in a subject in need thereof, the method comprising (a) detecting the level of CCR8 expressing cells in a sample obtained from the subject, detecting comprising: (i) contacting the sample with an isolated antibody or an antigen-binding fragment thereof that binds to human C-C motif chemokine receptor (CCR8), wherein the antibody or fragment thereof comprises a heavy chain variable (VH) region comprising an HCDR1 comprising the amino acid sequence of SEQ ID NO: 5, an HCDR2 comprising the amino acid sequence of SEQ ID NO: 6, an HCDR3 comprising the amino acid sequence of SEQ ID NO: 7; and a light chain variable (VL) region comprising an LCDR1 comprising the amino acid sequence of SEQ ID NO: 8, an LCDR2 comprising the amino acid sequence of SEQ ID NO: 9, and an LCDR3 comprising the amino acid sequence of SEQ ID NO: 10, under conditions sufficient and for a period of time to permit binding between the isolated antibody and/or the binding polypeptide and CCR8; (ii) detecting the level of CCR8 expressing cells in said sample; and (iii) associating the level of CCR8 expressing cells with a diseased microenvironment; and (b) administering to the subject an effective amount of a therapeutic anti-CCR8 agent comprising an antibody or antigen-binding fragment thereof that can specifically bind to CCR8.

In a ninth aspect, provided herein is a method of detecting CCR8 in a solid tumor sample obtained from a subject, comprising: (a) contacting the solid tumor sample with an isolated antibody or an antigen-binding fragment thereof that binds to human C-C motif chemokine receptor (CCR8), wherein the antibody or fragment thereof comprises a heavy chain variable (VH) region comprising an HCDR1 comprising the amino acid sequence of SEQ ID NO: 5, an HCDR2 comprising the amino acid sequence of SEQ ID NO: 6, an HCDR3 comprising the amino acid sequence of SEQ ID NO: 7; and a light chain variable (VL) region comprising an LCDR1 comprising the amino acid sequence of SEQ ID NO: 8, an LCDR2 comprising the amino acid sequence of SEQ ID NO: 9, and an LCDR3 comprising the amino acid sequence of SEQ ID NO: 10; under conditions sufficient and for a period of time to permit binding between the isolated antibody and/or the binding polypeptide and CCR8; and (b) detecting the level of CCR8 expressing cells in said solid tumor sample.

In a tenth aspect, provided herein is a method of selecting a subject having a solid cancer for treatment with a therapeutic anti-CCR8 agent, the method comprising: (a) obtaining a solid tumor sample from the subject; (b) detecting the level of CCR8 expressing cells in the solid tumor sample by performing a method of detecting CCR8 in a solid tumor sample provided herein; (c) comparing the level of the subject's CCR8 expressing cells with a threshold level of CCR8 expressing cells; and (d) selecting the subject for treatment with an anti-CCR8 agent if the level of the subject's CCR8 expressing cells exceed the threshold level of CCR8 expressing cells.

In an eleventh aspect, provided herein is a method of treating a subject having a solid cancer with a therapeutic anti-CCR8 agent, the method comprising: (a) selecting the subject for treatment by performing a method of selecting a subject having a solid cancer for treatment with a therapeutic anti-CCR8 agent provided herein; and (b) administering an effective amount of the therapeutic anti-CCR8 agent.

In a twelfth aspect, provided herein is a method of monitoring therapeutic efficacy of a treatment with a therapeutic anti-CCR8 agent in a subject having a solid cancer, the method comprising: (a) obtaining a solid tumor sample from the subject; (b) detecting the level of CCR8 expressing cells in the solid tumor sample by performing a method of detecting CCR8 in a solid tumor sample provided herein; and (c) comparing the detected level of the subject's CCR8 expressing cells with a threshold level of CCR8 expressing cells; wherein the treatment of the therapeutic anti-CCR8 agent has therapeutic efficacy if the detected level CCR8 expressing cells is depleted relative to the threshold level of CCR8 expressing cells.

In a thirteenth aspect, provided herein is a method of evaluating the prognosis of a subject having a solid cancer and undergoing treatment with a therapeutic anti-CCR8 agent, the method comprising: (a) obtaining a solid tumor sample from the subject; (b) detecting the level of CCR8 expressing cells in the solid tumor sample by a method of monitoring therapeutic efficacy of a treatment with a therapeutic anti-CCR8 agent in a subject having a solid cancer provided herein; and (c) comparing the detected level of the subject's CCR8 expressing cells with a threshold level of CCR8 or CCR8 expressing cells; wherein the subject has an improved prognosis if the detected level CCR8 expressing cells is depleted relative to the threshold level of CCR8 expressing cells.

In some embodiments of the twelfth of thirteenth aspects, the threshold level of CCR8 expressing cells is a baseline level of CCR8 expressing cells of the subject prior to receiving the initial treatment with the therapeutic anti-CCR8 agent, or any subsequent treatment with the therapeutic anti-CCR8 agent. In some embodiments, the CCR8 expressing cells are Treg cells and the detection measures the depletion of CCR8+ Treg cells.

In some embodiments of the seventh, eighth, ninth, tenth, eleventh, twelfth or thirteenth aspects, the solid cancer is chosen from squamous cell carcinoma, small cell lung cancer (SCLC), non-small cell lung cancer (NSCLC), squamous NSCLC, non-squamous NSCLC, head and neck cancer, breast cancer, cancer of the esophagus, gastric cancer, gastrointestinal cancer, cancer of the small intestine, liver cancer, hepatocellular carcinoma (HCC), pancreatic cancer (PAC), kidney cancer, renal cell carcinoma (RCC), bladder cancer, cancer of the urethra, cancer of the ureter, colorectal cancer (CRC), colon cancer, colon carcinoma, cancer of the anal region, endometrial cancer, prostate cancer, a fibrosarcoma, neuroblastoma, glioma, glioblastoma, germ cell tumor, pediatric sarcoma, sinonasal natural killer, melanoma, skin cancer, bone cancer, cervical cancer, uterine cancer, carcinoma of the endometrium, carcinoma of the fallopian tubes, ovarian cancer, carcinoma of the cervix, carcinoma of the vagina, carcinoma of the vulva, testicular cancer, cancer of the endocrine system, thyroid cancer, cancer of the parathyroid gland, cancer of the adrenal gland, sarcoma of soft tissue, cancer of the penis, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, solid tumors of childhood, environmentally-induced cancers, virus related cancers, cancers of viral origin, advanced cancer, unresectable cancer, metastatic cancer, refractory cancer, recurrent cancer, and any combination thereof.

In some embodiments of the eighth, tenth, eleventh, twelfth or thirteenth aspects, the therapeutic anti-CCR8 agent is selected from TPP-23411, TPP-29338, TPP-27454, TPP-31741, BMS-986340, LM-108, S-531011, FPA157, IPG-7236, ICP-B05, SRF-114, HBM1022, HFB1011, BAY-3375968, 10-1, ZL-1218, GB2101, PSB-114, dekitinug, IPG-A05, PM-1024, DT-7012, BCG-005, GNUV-202, CHS-3318, CTM-033, DT-7012, EGL-002, BGB-A3055, ABBV-514, ABT-863, or CHS-114.

In some embodiments of the eighth, tenth, eleventh, twelfth or thirteenth aspects, the therapeutic anti-CCR8 agent selected from azirkitug, cafelkibart, lanerkitug, imzokitug, denikitug, or enzelkitug.

In some embodiments of the eighth, tenth, eleventh, twelfth or thirteenth aspects, the therapeutic anti-CCR8 agent comprises (a) a heavy chain complementary determining region 1 (HCDR1) having the amino acid sequence of SEQ ID NO: 53, (b) an HCDR2 having the amino acid sequence of SEQ ID NO: 54, (c) an HCDR3 having the amino acid sequence of SEQ ID NO: 55, (d) a light chain complementarity determining region 1 (LCDR1) having the amino acid sequence of SEQ ID NO: 56, (e) an LCDR2 having the amino acid sequence of SEQ ID NO: 57, and (f) an LCDR3 having the amino acid sequence of SEQ ID NO: 58.

In some embodiments of the eighth, tenth, eleventh, twelfth or thirteenth aspects, the therapeutic anti-CCR8 agent comprises (a) a heavy chain variable region (VH) having at least 90%, 91%, 92%, 93%, 94%, 95%, 96%, 97%, 98%, 99%, or 100% sequence identity to the amino acid sequence of SEQ ID NO: 59; and (b) a light chain variable region (VL) having at least 90%, 91%, 92%, 93%, 94%, 95%, 96%, 97%, 98%, 99%, or 100% sequence identity to the amino acid sequence of SEQ ID NO: 60.

In some embodiments of the eighth, tenth, eleventh, twelfth or thirteenth aspects, the therapeutic anti-CCR8 agent comprises (a) a heavy chain variable region (VH) having the amino acid sequence of SEQ ID NO: 59; and (b) a light chain variable region (VL) having the amino acid sequence of SEQ ID NO: 60.

In some embodiments of the eighth, tenth, eleventh, twelfth or thirteenth aspects, the therapeutic anti-CCR8 agent comprises (a) a heavy chain having at least 90%, 91%, 92%, 93%, 94%, 95%, 96%, 97%, 98%, 99%, or 100% sequence identity to the amino acid sequence of SEQ ID NO: 61; and (b) a light chain having at least 90%, 91%, 92%, 93%, 94%, 95%, 96%, 97%, 98%, 99%, or 100% sequence identity to the amino acid sequence of SEQ ID NO: 62.

In some embodiments of the eighth, tenth, eleventh, twelfth or thirteenth aspects, the therapeutic anti-CCR8 agent comprises (a) a heavy chain having the amino acid sequence of SEQ ID NO: 61; and (b) a light chain having the amino acid sequence of SEQ ID NO: 62.

In some embodiments of the eighth, tenth, eleventh, twelfth or thirteenth aspects, the therapeutic anti-CCR8 agent is afucosylated.

In some embodiments of the eighth, tenth, eleventh, twelfth or thirteenth aspects, the therapeutic anti-CCR8 agent is denikitug.

In some embodiments of the first, second, third, fourth, fifth, sixth, seventh, eighth, ninth, tenth, eleventh, twelfth or thirteenth aspects, the sample is a tissue sample. In some embodiments, the tissue sample is a formalin-fixed paraffin-embedded (FFPE) tissue sample.

In some embodiments of the first, second, third, fourth, fifth, sixth, seventh, eighth, ninth, tenth, eleventh, twelfth or thirteenth aspects, the sample comprises a therapeutic anti-CCR8 antibody. In some embodiments, the therapeutic anti-CCR8 antibody binds to an epitope of the extracellular domain (ECD) of human CCR8. In some embodiments, the therapeutic anti-CCR8 antibody binds to an epitope of the amino acid sequence set forth in SEQ ID NO: 16, and wherein the epitope is different from that of the isolated antibody or antigen-binding fragment thereof that binds to human CCR8.

In some embodiments of the first, second, third, fourth, fifth, sixth, seventh, eighth, ninth, tenth, eleventh, twelfth or thirteenth aspects, the antibody or fragment thereof further comprises a detectable label.

### BRIEF DESCRIPTION OF THE DRAWINGS

FIG. 1 shows an exemplary cell pellet array (CPA) map indicating the level of CCR8 expression for the indicated cell type, including NSO wildtype (CCR8 negative), NSO huCCR8 (CCR8 high), HEK293 wildtype (CCR8 negative), HEK293 untagged CCR8 (CCR8 high), and Hut78 wildtype (CCR8 medium) cell pellets at various concentrations (0.2, 1.0, and 5 μg/mL) as used for anti-CCR8 antibody screening and depicted in FIGS. 2A-2E. As used in the figure: NSO WT=NSO wildtype (CCR8 negative) cells; NSO huCCR8=NSO huCCR8-expressing (CCR8 high) cells; Hut78=Hut78 wildtype (CCR8 medium) cells; HEK293 untagged huCCR8=HEK293 untagged huCCR8-expressing (CCR8 high) cells; and HEK293=HEK293 wildtype (CCR8 negative) cells.

FIGS. 2A-2E show exemplary results of IHC detection of CCR8 on NSO wildtype (CCR8 negative), NSO huCCR8 (CCR8 high), HEK293 wildtype (CCR8 negative), HEK293 untagged CCR8 (CCR8 high), and Hut78 wildtype (CCR8 medium) cell pellets using five different anti-CCR8 antibodies: BD 566379 (Clone 433H; FIG. 2A), used at 0.2 μg/mL; Abcam ab140796 (rabbit polyclonal; FIG. 2B) used at 5 μg/mL; BioLegend 360602 (Clone L263G8; FIG. 2C), used at 0.2 μg/mL; R&D MAB1429 (Clone 191704; FIG. 2D), used at 5 μg/mL; and Sigma HPA042383 (FIG. 2E), used at 0.2 μg/mL. As used in the figure: NSO WT=NSO wildtype (CCR8 negative) cells; NSO+huCCR8=NSO huCCR8-expressing (CCR8 high) cells; HUT78=Hut78 wildtype (CCR8 medium) cells; HEK293+huCCR8=HEK293 untagged huCCR8-expressing (CCR8 high) cells; and HEK293=HEK293 wildtype (CCR8 negative) cells.

FIG. 3 shows exemplary results of the immunohistochemistry (IHC) detection of CCR8 in human head and neck squamous cell carcinoma Hu-P-002132 (panel A) and Hu-P-002140 (panel B) formalin-fixed paraffin-embedded (FFPE) tissue samples using BD 566379 (Clone 433H).

FIG. 4 shows exemplary results of IHC detection of CCR8 on NSO wildtype (CCR8 negative), NSO huCCR8 (CCR8 high), HEK 293 wildtype (CCR8 negative), HEK293 untagged CCR8 (CCR8 high), Hut78 wildtype (CCR8 medium), and DT 40 (CCR8 medium-high) cell pellets using unconjugated 433H antibody. The staining was performed at antibody concentrations of 0.1 μg/mL of the unconjugated BD 566379 (Clone 433H) antibody. As used in the figure: NSO=NSO wildtype (CCR8 negative) cells; NSO-huCCR8 OE=NSO huCCR8-expressing (CCR8 high) cells; Hut78=Hut78 wildtype (CCR8 medium) cells; HEK293-huCCR8 OE=HEK293 untagged huCCR8-expressing (CCR8 high) cells; DT40=DT40 (CCR8 medium-high) cells, and HEK293=HEK293 wildtype (CCR8 negative) cells.

FIG. 5 shows exemplary results of IHC detection of CCR8 in human thymus FFPE tissue samples (Hu-P-003724) using unconjugated BD 566379 (Clone 433H) antibody. The staining was performed at antibody concentrations of 0.1 μg/mL, 0.2 μg/mL, 0.5 μg/mL, or 1 μg/mL of the unconjugated BD 566379 (Clone 433H) antibody.

FIG. 6 shows exemplary results of IHC detection of CCR8 in human head and neck squamous cell carcinoma (Hu-P-003302), triple-negative breast cancer (Hu-P-003650), and non-small cell lung cancer non-squamous (Hu-P-003658) FFPE tissue samples (Hu-P-003724) using unconjugated BD 566379 (Clone 433H) antibody. The staining was performed at antibody concentrations of 0.2 μg/mL of the unconjugated BD 566379 (Clone 433H) antibody.

FIG. 7 shows exemplary results of a competition assay using antibody 7-B16, which targets a N-terminal epitope (also referred to as the “extracellular domain” or “ECD” of CCR8). CCR8-expressing HEK293T (“293T”) cells, treated and not treated with 7-B16, were stained with BD 566379 (Clone 433H) antibody, or an IgG control. Wildtype HEK293T cells were used as a negative control.

### DETAILED DESCRIPTION

### Definitions

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

As is understood by one skilled in the art, reference to “about” a value or parameter herein includes (and describes) embodiments that are directed to that value or parameter per se. For example, description referring to “about X” includes description of “X”.

The term “affinity” as used herein in reference to an antibody or binding polypeptide refers to the strength of the sum total of noncovalent interactions between a single binding site of a molecule (for example, an antibody or binding polypeptide) and its binding partner (for example, an antigen). The affinity of a molecule X for its partner Y can generally be represented by the dissociation constant (KD). Affinity can be measured by common methods known in the art (such as, for example, ELISA KD, KinExA, bio-layer interferometry (BLI), and/or surface plasmon resonance devices (such as a BIAcore® device), including those described herein).

The term “antibody” (Ab) refers to an immunoglobulin molecule (e.g. without limitation human IgG1, IgG2, IgG3, IgG4, IgM, IgD, IgE, IgA1, IgA2, mouse IgG1, IgG2a, IgG2b, IgG2c, IgG3, IgA, IgD, IgE or IgM, rat IgG1, IgG2a, IgG2b, IgG2c, IgA, IgD, IgE or IgM, rabbit IgA1, IgA2, IgA3, IgE, IgG, IgM, goat IgA, IgE, IgG1, IgG2, IgE, IgM or chicken IgY) that binds to, or is immunologically reactive with, a particular antigen. The term antibody includes, but is not limited to, fragments that are capable of binding to an antigen, such as antigen-binding regions and/or effector regions of the antibody, e.g., Fab, Fab′, F(ab′)₂, Fv, scFv, (scFv)₂, single chain antibody molecule, dual variable region antibody, single variable region antibody, linear antibody, V region, a multispecific antibody formed from antibody fragments, F(ab)₂ (including a chemically linked F(ab′)₂), Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled). Accordingly, it is understood that the term “antibody” also encompasses any antigen binding fragments thereof, and thus the terms “antibody”, “antibody fragment” and “antigen-binding fragment thereof” are used interchangeably herein. Depending on the respective context, the term antibody may also refer to any proteinaceous binding molecule with immunoglobulin-like function, such as a “binding polypeptide” as described herein.

The term antibody also includes, specifically covers, but is not limited to, for example polyclonal antibodies, monoclonal antibodies (including agonist, antagonist, neutralizing antibodies, full length monoclonal antibodies), antibody compositions with polyepitopic or monoepitopic specificity, recombinantly produced antibodies, monospecific antibodies, multispecific antibodies (including bispecific antibodies), synthetic antibodies, chimeric antibodies, humanized antibodies, or human versions of antibodies having full length heavy and/or light chains. As the term antibody includes chimeric antibodies, humanized antibodies, and antibodies of various species such as mouse, human, cynomolgus monkey, etc. are also encompassed by an antibody described herein. Furthermore, for all antibody constructs provided herein, variants having the sequences from other organisms are also contemplated. Thus, if a human version of an antibody is disclosed, one of skill in the art will appreciate how to transform the human sequence based antibody into a mouse, rat, cat, dog, horse, etc. sequence.

A variable (V) region domain may be any suitable arrangement of immunoglobulin heavy (VH) and/or light (VL) chain variable domains. For example, the present disclosure also includes tetrameric antibodies comprising two heavy chain and two light chain molecules, an antibody light chain monomer, and an antibody heavy chain monomer. Thus, for example, the V region domain may be dimeric and contain VH-VH, VH-VL, or VL-VL dimers that bind CCR8. If desired, the VH and VL chains may be covalently coupled either directly or through a linker to form a single chain Fv (scFv) (e.g., an antibody fragment described herein). Another form of an antibody fragment is a peptide comprising one or more complementarity determining regions (CDRs) of an antibody. CDRs (also termed “minimal recognition units” or “hypervariable region”) can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). The more highly conserved portions of the variable domains are called the framework (FR). As is known in the art, the amino acid position/boundary delineating a hypervariable region of an antibody can vary, depending on the context and the various definitions known in the art. Antibody fragments, regions and/or domains are further described in detail throughout the present disclosure. Antibody fragments may be incorporated into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23(9):1126-1136, 2005).

The term “antibody-dependent cell-mediated cytotoxicity” or “ADCC” refers to a form of cytotoxicity in which secreted Ig bound onto Fc receptors (FcRs) present on certain cytotoxic cells (for example NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 or 6,737,056 (Presta), may be performed. Useful effector cells for such assays include PBMC and NK cells. Alternatively, or additionally, ADCC activity of the molecule of interest may be assessed in vivo, for example, in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, for example, in U.S. Pat. Nos. 7,923,538, and 7,994,290.

The term “antibody-dependent cellular phagocytosis” or “ADCP” as used herein refers to the mechanism by which antibody-opsonized target cells activate the FcγRs on the surface of immune cells (e.g., macrophages to induce phagocytosis), resulting in internalization and degradation of the target cell. For ADCP, binding to macrophages as effector cells typically occurs via the interaction of the antibodies FC part with FcγRIIa (CD32a) expressed by macrophages.

The term “complement-dependent cytotoxicity” or “CDC” as used herein refers to an effector function of IgG and IgM antibodies when they are bound to a surface antigen on a target cell (e.g. T regulatory cell), the classical complement pathway is triggered by bonding protein C1q to these antibodies, resulting in formation of a membrane attack complex (MAC) and target cell lysis. Complement system is efficiently activated by antibodies, such as human IgG1, IgG3 and IgM antibodies.

The term “CCR8” as used herein refers to C-C chemokine receptor type 8. Synonyms for CCR8 are inter alia CC-CKR-8, CCR-8, CDw198, CKRL1, CMKBR8, CMKBRL2, GPRCY6, CY6, TERI. The CCR8 protein is encoded by the gene CCR8 (NCBI gene ID 1237). The term includes human, murine, rabbit, and Rhesus macaque CCR8 protein and further mammalian and non-mammalian homologues. Different isoforms and variants, including splice variants or allelic variants. may exist for the different species and are all comprised by the term CCR8. Also comprised are CCR8 molecules before and after maturation, e.g., independent of cleavage of one or more pro-domains. In addition, synthetic variants of the CCR8 protein may be generated and are comprised by the term CCR8. The protein CCR8 may furthermore be subject to various modifications, e.g., synthetic or naturally occurring modifications, such as post translational modifications. Recombinant human CCR8 is commercially available or can be manufactured by any suitable technique. CCR8 is a receptor for the chemokine CCL1/SCYA1/I-309.

The term “CDR” as used in reference to an antibody provided herein describes a complementarity determining region as defined by at least one manner of identification to one of skill in the art. In some instances, CDRs can be defined in accordance with any one of the well-known schemes in the art, such as the Chothia numbering scheme, the Kabat numbering scheme, the IMGT numbering scheme, the CCG numbering scheme, the North numbering scheme, the Honeggar numbering scheme, or a combination of Kabat, Chothia, IMGT, CCG, North, and/or Honeggar. The various CDRs within an antibody can be designated by their appropriate number and chain type, including, without limitation as: a) CDR-L1, CDR-L2, CDR-L3, CDR-H1, CDR-H2, and CDR-H3; b) CDRL1, CDRL2, CDRL3, CDRH1, CDRH2, and CDRH3; c) LCDR-1, LCDR-2, LCDR-3, HCDR-1, HCDR-2, and HCDR-3; d) LCDR1, LCDR2, LCDR3, HCDR1, HCDR2, and HCDR3; e) VH CDR1, VH CDR2, VH CDR3, VL CDR1, VL CDR2, VL CDR3; etc. The term “CDR” is used herein to also encompass HVR or a “hyper variable region”, including hypervariable loops. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).)

The term “chimeric antibody” as used herein refers to an antibody in which a portion of the heavy and/or light chain is derived from a particular source or species, while at least a part of the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some instances, a chimeric antibody comprises at least one mouse variable region and at least one human constant region. In some instances, a chimeric antibody comprises at least one cynomolgus variable region and at least one human constant region. In some instances, all of the variable regions of a chimeric antibody are from a first species and all of the constant regions of the chimeric antibody are from a second species. The chimeric construct can also be a functional fragment, as noted above.

The term “conjugate” as used herein, refers to antibody or binding polypeptide that is chemically linked to a second agent, such as a therapeutic or cytotoxic agent. The term “agent” includes a chemical compound, a mixture of chemical compounds, a biological macromolecule, or an extract made from biological materials.

The term “detect” and grammatical equivalents thereof as used herein encompasses quantitative or qualitative indication of the presence, absence or abundance of the item being detected. Relatedly, the term “quantify” and grammatical equivalents thereof as used herein encompasses the quantitative indication of the abundance of the item being detected.

The term “dissociation constant” (KD) as used herein is measured by molar units (M) and refers to the concentration of the binder/antibody at which half of the target proteins are occupied at equilibrium. The smaller the dissociation constant is, the higher is the affinity between the binder or antibody and its target. In some instances, antibodies and/or binding polypeptides provided herein have a target affinity of at least 10-7 M (as KD value), more preferably of at least 10-8 M, even more preferably in the range from 10-9 M to 10-11 M. The KD values can be preferably determined by means of surface plasmon resonance spectroscopy, e.g. as described elsewhere herein. Where assay conditions were found to influence the determined KD, the assay setup with the least standard deviation shall be used.

The term “effective amount” or “therapeutically effective amount” as used interchangeably herein, refer to an amount sufficient to achieve a particular biological result or to modulate or ameliorate a symptom in a subject, or the time of onset of a symptom, typically by at least about 10%; usually by at least about 20%, preferably at least about 30%, or more preferably at least about 50%. Efficacy of the use of an antibody in cancer therapy can be assessed based on the change in tumor burden. Both tumor shrinkage (objective response) and time to the development of disease progression are important endpoints in cancer clinical trials. Standardized response criteria, known as RECIST (Response Evaluation Criteria in Solid Tumors), were published in 2000. An update (RECIST 1.1) was released in 2009. RECIST criteria are typically used in clinical trials where objective response is the primary study endpoint, as well as in trials where assessment of stable disease, tumor progression or time to progression analyses are undertaken because these outcome measures are based on an assessment of anatomical tumor burden and its change over the course of the trial. An effective amount for a particular subject may vary depending on factors such as the condition being treated, the overall health of the subject, the method, route, and dose of administration and the severity of side effects. When in combination, an effective amount is in ratio to a combination of components and the effect is not limited to individual components alone.

The term “epitope” as used herein refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example, an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

The term “heavy chain” or “HC” as used in reference to an antibody provided herein describes a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “heavy chain variable region” or “VH” as used in reference to an antibody provided herein describes a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FR1 and/or at least a portion of an FR4.

The term “human antibody” as used herein encompasses antibodies produced in humans, antibodies produced in non-human animals that comprise human immunoglobulin genes, such as XenoMouse® mice, and antibodies selected using in vitro methods, such as phage display (Vaughan et al., Nature Biotechnology, 14:309-314 (1996); Sheets et al., 1998, Proc. Natl. Acad. Sci. (USA) 95:6157-6162; Hoogenboom and Winter, J. Mol. Biol., 227:381 (1991); Marks et al., J. Mol. Biol., 222:581 (1991)), wherein the antibody repertoire is based on a human immunoglobulin sequence. The term “human antibody” denotes the genus of sequences that are human sequences. Thus, the term is not designating the process by which the antibody was created, but the genus of sequences that are relevant.

The term “humanized antibody” as used herein refers to an antibody in which at least one amino acid in a framework region of a non-human variable region has been replaced with the corresponding amino acid from a human variable region. In some instances, a humanized antibody comprises at least one human constant region or fragment thereof. In some instances, a humanized antibody is an antibody fragment, such as Fab, an scFv, a (Fab′)2, etc. The term humanized also denotes forms of non-human (for example, murine) antibodies that are chimeric immunoglobulins, immunoglobulin chains, or fragments thereof (such as Fv, Fab, Fab′, F(ab′)2 or other antigen-binding subsequences of antibodies) that contain minimal sequence of non-human immunoglobulin. Humanized antibodies can include human immunoglobulins (recipient antibody) in which residues from a complementary determining region (CDR) of the recipient are substituted by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody can comprise residues that are found neither in the recipient antibody nor in the imported CDR or framework sequences, but are included to further refine and optimize antibody performance. In some instances, the humanized antibody can comprise substantially all of at least one, and typically two, variable domains, in which all or substantially all of the CDR regions correspond to those of a non-human immunoglobulin and all or substantially all of the FR regions are those of a human immunoglobulin consensus sequence. In some instances, the humanized antibody can also comprise at least a portion of an immunoglobulin constant region or domain (Fc), typically that of a human immunoglobulin. Other forms of humanized antibodies have one or more CDRs (CDR L1, CDR L2, CDR L3, CDR H1, CDR H2, and/or CDR H3) which are altered with respect to the original antibody, which are also termed one or more CDRs “derived from” one or more CDRs from the original antibody. As will be appreciated, a humanized sequence can be identified by its primary sequence and does not necessarily denote the process by which the antibody was created.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”. An isolated polypeptide or antibody may however be immobilized, e.g. on beads or particles, e.g. via a suitable linker.

The terms “label” and “detectable label” as used herein mean a moiety or molecule attached to an antibody, a binding polypeptide or its analyte to render a reaction (for example, binding) between the members of the specific binding pair, detectable. The labeled member of the specific binding pair is referred to as “detectably labeled.” In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, 3H, 14C, 35S, 90Y, 99Tc, 111In, 125I, 131I, 177Lu, 166Ho, or 153Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety. Detectable labels are further described herein.

The term “light chain” or “LC” as used in reference to an antibody provided herein describes a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

The term “light chain variable region” or “VL” as used in reference to an antibody provided herein describes a region comprising at least three light chain CDRs. In some embodiments, the light chain variable region includes the three CDRs and at least FR2 and FR3. In some embodiments, the light chain variable region includes at least light chain LCDR1, framework (FR) 2, LCDR2, FR3, and LCDR3. For example, a light chain variable region may comprise light chain CDR1, framework (FR) 2, CDR2, FR3, and CDR3. In some embodiments, a light chain variable region also comprises at least a portion of an FR1 and/or at least a portion of an FR4.

The term “linker” as used herein refers to any molecule enabling a direct topological connection between two moieties. A moiety may be inter alia a polypeptide, a protein, an antibody, an antibody fragment, a cytotoxic moiety, a binding moiety, a moiety for detection such as a fluorophore, a moiety for immobilization or retrieval such as beads or magnetic beads, a reactive moiety, or any other molecule. The two moieties may be of the same type or different. Linkers may be part of conjugates and may even contribute to their function. For instance, for a conjugate comprising a polypeptide and a biotin, the presence of a spacer of approximately 4 Å (5 atoms) between the carboxy group of the biotin and the 1st bulky amino acid of the peptide allows the biotin to reach the (strept)avidin binding pocket. Any suitable can be selected, including linkers selected based on the moieties which shall be connected. The linker length typically ranges between 4 atoms and more than 200 atoms. Linkers exceeding 60 atoms in length generally comprise a population of compounds having an average length.

The term “mediate” as used herein means to bring about a result. For example, when in reference to an agent (e.g., an anti-CCR8 binding agent) or a cell (e.g., a Treg cell), the term mediate describes the action of the agent or cell to induce an action and/or ability of the agent or cell to affect another agent, cell process, or mechanism to induce an outcome.

The term “monoclonal antibody” as used herein refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., Nature 348:552-554 (1990), for example.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (e.g., conservative substitutions), to the native sequence, as long as the protein maintains the desired activity. These modifications may be deliberate, as through site-directed mutagenesis, or may be accidental, such as through mutations of hosts, which produce the proteins or errors due to PCR amplification. Where generic reference is to a protein or gene from a certain species such as mouse, the analogue from human shall likewise be meant, if not stated otherwise or obviously incompatible.

The terms “polynucleotide”, “nucleic acid molecule”, and “nucleic acid” may be used interchangeably, and refer to a polymer of nucleotides. Such polymers of nucleotides may contain natural and/or non-natural nucleotides, and include, but are not limited to, DNA, RNA, and PNA. “Nucleotide sequence” refers to the linear sequence of nucleotides that comprise the polynucleotide.

The term “Regulatory T cells”, “Tregs”, “Treg cells”, or “suppressor T cells” used interchangeable herein describes a subpopulation of T cells that are immunosuppressive and generally suppress or downregulate induction and proliferation of effector T cells. Tregs express CD4, FOXP3, and CD25 (IL-2 receptor α-chain). Human Foxp3+CD4+ T cells have been divided into three subfractions based upon the expression level of Foxp3 and the cell surface molecules CD25 and CD45RA. The Foxp3hiCD45RA-CD25hi and Foxp3loCD45RA+CD25lo phenotypes correspond to immunosuppressive Treg cells, whereas the Foxp3loCD45RA-CD25lo fraction marks activated T effector (Teff) cells without immunosuppressive activity. In addition, Treg cells from cancer patients, as compared to those in healthy subjects, are usually characterized by a distinct expression profile of chemokine receptors, such as CCR4, CXCR4, and CCR5, which facilitates their migration into tumors in response to the corresponding chemokine ligands derived from tumor microenvironment. See, e.g., Liu, et al., FEBS J283(14):2731-48, (2016) and Miyara, et al., Immunity 30, 899-911 (2009).

Under physiological conditions, Treg cells maintain immunological tolerance. During an immune response, Treg cells stop T cell-mediated immunity and suppress auto-reactive T cells that have escaped negative selection within the thymus. Treg cells can also suppress other types of immune cells such as NK cells and B cells. Adaptive Treg cells (called Th3 or Tr cells) are thought to be generated during an immune response.

Treg cells furthermore play an important role in immune escape by suppressing antitumor immunity, thereby providing an environment of immune tolerance. T cells that recognize cancer cells are often present in large numbers in tumors, but their cytotoxic function is suppressed by nearby immune-suppressor cells. Tregs are abundant in many different cancers, are highly enriched in the tumor microenvironment, and are well known for their role in tumor progression. Tregs that reside in diseased microenvironments, e.g., tumor microenvironments, are described further herein.

The term “specifically binds” as used herein in reference to an antigen or epitope describes an antibody or a binding polypeptide that reacts or associates more frequently, more rapidly, with greater selectivity, duration and/or with greater affinity with a particular antigen or epitope than it does with alternative antigens or epitopes. For example, an antibody that specifically binds to a certain CCR8 epitope (e.g., an intracellular C-terminal CCR8 epitope) is an antibody that binds this epitope with greater affinity, avidity, selectivity, more readily, and/or with greater duration than it binds to other CCR8 epitopes or non-CCR8 epitopes. It is also understood by reading this definition that, for example, an antibody (or antigen-binding fragment thereof) that specifically binds to a first target may or may not specifically bind to a second target. As such, “specific binding” does not necessarily require (although it can include) exclusive binding.

The term “subject” as used herein to refers to an animal; for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, a “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

The term “threshold level” refers generally to an assay cutoff value that is used to assess diagnostic/prognostic/therapeutic efficacy results by comparing the assay results against the predetermined cutoff value, already has been linked or associated with various clinical parameters (for example, severity of disease, progression/nonprogression/improvement, etc.). While the present disclosure may provide exemplary threshold levels, it is well-known that cutoff values may vary depending on the nature of the immunoassay (for example, antibodies employed, etc.). It further is well within the skill of one of ordinary skill in the art to adapt the disclosure herein for other immunoassays to obtain immunoassay-specific threshold levels for those other immunoassays based on this disclosure. Whereas the precise value of the predetermined cutoff value may vary between assays, correlations as described herein (if any) may be generally applicable.

The term “treatment” as used herein is an approach for obtaining beneficial or desired clinical results. The term covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder. In the context of cancer, the term “treating” includes any or all of: inhibiting growth of cancer cells, inhibiting replication of cancer cells, lessening of overall tumor burden and ameliorating one or more symptoms associated with the disease.

The term “tumor infiltrating”, “tumoral”, “intra-tumoral”, or “intratumoral” as used herein in the context of cells, structures, proteins, antibodies, or markers refers to their localization within the tumor tissue. Cells which are “positive” or “+” for a certain marker or protein are cells characterized by substantial expression of that marker or protein. Marker or protein expression can be determined and quantified as known in the art, e.g. to define different cell populations. For the characterization of (immune) cell populations, the marker expression can be determined by FACS or using any other technique described herein.

The term “tumor infiltrating Treg cells” and equivalents thereof as used herein refers to Tregs that express CCR8. Without being bound by theory, it is postulated that CCR8 is expressed with much higher prevalence and at higher levels on the surface of cancer-resident Tregs compared to circulating or normal tissue Tregs and conventional T effector (Teff) cells. Treg cell infiltration in solid tumors is associated with poor clinical outcome, and Tregs suppress the anti-cancer immune response through inhibition of Teff cell cytotoxicity.

The term “vector” as used herein describes a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, 0-galactosidase). In some instances, a vector provided herein is an expression vector which is used to express a polypeptide of interest in a host cell.

### Overview

The present disclosure provides anti-CCR8 binding agents (e.g., antibodies, fragments thereof, binding polypeptides, and any modified derivatives or variants of the same) that bind to CCR8, and in some embodiments, specifically to a CCR8 polypeptide, CCR8 polypeptide fragment, a CCR8 peptide or a CCR8 epitope comprised in CCR8. In some embodiments, a CCR8 binding agent (e.g., an antibody), such as a human CCR8 binding agent, can bind to CCR8 expressed on the surface of a mammalian (e.g., human) cell, including a CCR8 expressing cell. In some embodiments, described herein is a CCR8 binding agent (e.g., an antibody) that binds to CCR8, such as human CCR8 or portions thereof. In some embodiments, CCR8 is a human CCR8. In some embodiments, a CCR8 binding agent is a human CCR8 binding agent (e.g., an antibody that binds to human CCR8).

In some embodiments, a CCR8 binding agent (e.g., an antibody) binds a CCR8 epitope of a CCR8 expressing cell (e.g., a target CCR8 epitope). In some embodiments, a CCR8 binding agent (e.g., an antibody) selectively binds a CCR8 epitope of a CCR8 expressing cell over an epitope of any one or combinations of CCR1, CCR2, CCR3, CCR4, CCR5, CCR6, CCR7, and/or CCR9 (e.g., a target CCR8 epitope).

CCR8 as described herein can be human CCR8 having an amino acid sequence as set forth in SEQ ID NO: 1 (UniProt Identifier P51685). However, CCR8 from any species corresponding to the species of any subject in compositions and methods described herein. For example, CCR8 as described herein can be mouse CCR8 (Mus musculus) having an amino acid sequence as set forth in SEQ ID NO: 2 (UniProt Identifier P56484); monkey CCR8 (Macaca mulatta) having an amino acid sequence as set forth in SEQ ID NO: 3 (UniProt Identifier 097665); or a rabbit CCR8 (Oryctolagus cuniculus) having an amino acid sequence as set forth in SEQ ID NO: 4 (Uniprot Identifier G1T2B2).

Specifically, CCR8 encompassed by the present disclosure has been shown to be selectively upregulated in tumor-resident regulatory T cells (Tregs or Treg cells) in multiple cancers (De Simone et al., Immunity, 45(5): 1135-1147 (2016); Plitas et al. Immunity. 45(5):1122-1134 (2016), and selectively expressed in the majority of tumor-infiltrating Tregs (WO 2021/194942) which suppress the anti-cancer immune response through inhibition of immune cell cytotoxicity. It has also been found that CCR8 expression has the highest correlation with FOXP3 (master transcriptional regulator of Tregs) in most cancer types and is expressed on the most activated and suppressive subset of FOXP3^{hi} tumor Tregs (e.g., immunosuppressive CD4+FOXP3^{high} Tregs), and can be associated with poor clinical outcome (see e.g., Plitas et al. Immunity. 45(5):1122-1134 (2016); Wang et al., Nat Immunol. (9):1220-1230 (2019); WO 2021/194942). On the other hand. CCR8 is rarely observed on Tregs and effector T cells (Teffs or Teff cells) in the peripheral blood (WO 2021/194942); on FOXP3^{mid} and FOXP3^{neg} CD8 and CD4 effector T cells in patient tumors, or on tumor-infiltrating CD4+ T cells and CD8+ T cells (WO 2021/194942). Thus, depletion of CCR8 expressing immune cells, or depletion of CCR8 expression would likely not significantly alter the peripheral Treg compartment.

A CCR8-related disease, disorder, or condition as encompassed by the present disclosure refers to refer to any disease, disorder or condition that is characterized by aberrant, upregulated, or selective expression of CCR8, and/or alternatively any disease, disorder, or condition in which it is desirable to deplete the expression of CCR8 and/or CCR8 expressing cells. A CCR8-related disease includes a disease, disorder or condition that is characterized by or associated with immunosuppressive activity of Treg cells, decreased ADCC activity of immune cells, decreased ADCP activity of immune cells, and/or decreased CDC activity, in a diseased microenvironment, such as a tumor microenvironment. In some embodiments, a CCR8-related disease is one in which is characterized by or associated with resistance to cancer treatments and/or decreased anti-tumor immunity. Thus, targeting a CCR8-related disease, disorder, or condition, such as diseased microenvironment characterized by CCR8 expression and/or CCR8 expressing cells, would likely also ameliorate resistance to cancer treatments and/or anti-tumor immunity.

In some embodiments, anti-CCR8 binding agents providing herein bind, or specifically bind to a target CCR8 polypeptide, CCR8 polypeptide fragment, a CCR8 peptide or a CCR8 epitope comprised in CCR8. In some embodiments, anti-CCR8 binding agents providing herein specifically bind to a target CCR8 epitope. The target epitope may be continuous or non-continuous, and may be determined by a method known to a person of ordinary skill, including flow cytometry of bound antibody to peptides, hydrogen-deuterium exchange, alanine scanning, and/or x-ray crystallography. The target epitope can comprise or consist of amino acid residues that are determined by anti-CCR8 binding agent binding to CCR8 as described herein. The target epitope can be an epitope comprising or consisting of amino acid residues that are determined by epitope binning. The target epitope can be an epitope comprising or consisting of amino acid residues that are determined by anti-CCR8 binding agent binding to CCR8 peptide-nanobody complexes. The target epitope can be an epitope comprising or consisting of amino acid residues that are determined by screening anti-CCR8 binding agent binding to CCR8 by phage display. The target epitope can be an epitope comprising or consisting of amino acid residues that are determined by in silico screening anti-CCR8 binding agent binding to CCR8 by computer learning models. The target epitope can be an epitope comprising or consisting of amino acid residues that are determined by anti-CCR8 binding agent binding to CCR8 in a non-competitive assay. The target epitope can be an epitope comprising or consisting of non-conserved amino acid residues as determined by multiple sequence alignment of CCR1-9.

In some embodiments, a desired target epitope is an epitope that targeting of the same will limit cross-reactivity with other receptors in the chemokine receptor family (e.g., CCR1, CCR2, CCR3, CCR4, CCR5, CCR6, CCR7, and CCR9). In some embodiments, a desired target epitope is an epitope of CCR8 that is different from the epitope of CCR8 that a therapeutic anti-CCR8 antibody binds to.

The compositions and methods disclosed herein are based at least in part on the recognition that, with the emergence of therapeutic anti-CCR8 antibodies for the treatment of cancer, there is a need to reliably and specifically detect and monitor CCR8 in a subject having received a therapeutic anti-CCR8 antibody. There is limited understanding of the targeting of CCR8 by Treg depleting therapeutic antibodies currently in development as anticancer treatments. While CCR8 is known to be activated by the endogenous C-C motif chemokine ligand 1 (CCL1) and coupled to the inhibitory signaling protein Gi, limited information is available about its molecular structure and activation mechanism, which further limits its targeting and inhibition by antibodies. While detection methods and reagents for CCR8 have been developed, it has remained difficult to accurately detect CCR8 is tissue samples in the presence of a therapeutic anti-CCR8 antibody in the sample that can block binding of CCR8 by detection reagents. The compositions and methods disclosed herein provide a way to reliably and specifically detect and monitor CCR8 and cells expressing CCR8 in tissue samples, even in the presence of a therapeutic anti-CCR8 antibody in the sample.

In some embodiments, the target epitope is not an epitope present in any one of CCR1-7 or 9. In some embodiments, the target epitope is not an epitope present in CCR1. In some embodiments, the target epitope is not an epitope present in CCR2. In some embodiments, the target epitope is not an epitope present in CCR3. In some embodiments, the target epitope is not an epitope present in CCR4. In some embodiments, the target epitope is not an epitope present in CCR5. In some embodiments, the target epitope is not an epitope present in CCR6. In some embodiments, the target epitope is not an epitope present in CCR7. In some embodiments, the target epitope is not an epitope present in CCR9. In some embodiments, the target epitope does not comprise or consist of any one of the amino acid sequences set forth in TABLE 1.1. In some embodiments, the target epitope does not comprise or consist of an epitope of any one of the amino acid sequences set forth in TABLE 1.1.

In some embodiments, the target epitope comprises an amino acid sequence that is about 60% about 65% about 70% about 75% about 80% about 85% about 90% about 95%, about 97%, about 98%, about 99% identical or is identical to any one of the sequences set forth in TABLE 1. In some embodiments, the target epitope comprises an amino acid sequence that is 1 amino acid, 2 amino acids, 3 amino acids, 4 amino acids, 5 amino acids, 6 amino acids, 7 amino acids, 8 amino acids, 9 amino acids, 10 amino acids, 11 amino acids, 12 amino acids, 13 amino acids, 14 amino acids, 15 amino acids, 16 amino acids, 17 amino acids, 18 amino acids, 19 amino acids, or 20 amino acids which are contiguous or non-contiguous and selected from any one of the sequences set forth in TABLE 1.

In some embodiments, anti-CCR8 binding agents provided herein can have a strong binding affinity and/or specificity for CCR8. In some embodiments, the anti-CCR8 binding agents provided herein can have a unique and selective binding to CCR8, for example, which does not interfere with other, commercially available anti-CCR8 binding agents. In some embodiments, anti-CCR8 binding agents provided herein are useful for the detection of CCR8 expression and/or CCR8 expressing cells. Accordingly, in some embodiments, anti-CCR8 binding agents provided herein are useful for patient diagnosis, selection for treatment of a disease associated with CCR8 expression, and/or monitoring the therapeutic efficacy of the treatment and/or the prognosis of a subject receiving such treatment. In some embodiments, anti-CCR8 binding agents provided herein are useful for the detection and/or quantification of CCR8 expression and/or expressing cells in a cancer sample. In some embodiments the sample is a solid tumor biopsy sample. In some embodiments the sample is a fixed tumor tissue sample. In some embodiments the tissue sample is formalin fixed. In some embodiments, the tissue sample is a formalin-fixed paraffin embedded (FFPE) tissue sample. In some embodiments, the sample comprises a therapeutic anti-CCR8 antibody. In some embodiments, the therapeutic anti-CCR8 antibody binds to an epitope of the extracellular domain (ECD) of human CCR8. In some embodiments, the therapeutic anti-CCR8 antibody binds to an epitope of the amino acid sequence set forth in SEQ ID NO: 16, and wherein the epitope is different from that of the isolated antibody or antigen-binding fragment thereof that binds to human CCR8. In some embodiments, expression of CCR8 is on Treg cells, and/or CCR8 expressing cells are CCR8+ Treg cells. In some embodiments, anti-CCR8 binding agents provided herein are useful for the detection and/or quantification of CCR8 expression and/or CCR8 expressing cells in a sample of a subject having received an anti-CCR8 antibody therapy. In some embodiments, anti-CCR8 binding agents provided herein are useful for the detection and/or quantification of CCR8 expression and/or CCR8 expressing cells in a sample in the presence of a therapeutic anti-CCR8 antibody. Accordingly, in some embodiments, anti-CCR8 binding agents provided herein are useful for the monitoring the progress of a subject receiving treatment of a disease associated with CCR8 expression and/or CCR8 expressing cells.

In some embodiments, anti-CCR8 binding agents provided herein activate immune responses by certain immune cells, and/or mediate the activity of and/or deplete the amount of other immune cells, in a diseased microenvironment associated with CCR8 expression, such as a tumor microenvironment. In some embodiments, anti-CCR8 binding agents provided herein induce cancer cell killing by activating immune responses by immune cells (e.g., effector T-cells), and/or mediating activity of and/or depleting the amount of immune-suppressive cells (e.g., Treg cells) in a diseased microenvironment associated with CCR8 expression, such as a tumor microenvironment. In some embodiments the anti-CCR8 binding agents provided herein deplete the amount of tumor resident CCR8 positive Treg cells. Accordingly, in some embodiments, anti-CCR8 binding agents provided herein are useful for the treatment of a disease associated with CCR8 expression, such as cancer.

Examples of anti-CCR8 binding agents are described in further detail herein.

### I. Anti-CCR8 Binding Agents

Provided herein are anti-CCR8 binding agents or uses thereof. An anti-CCR8 binding agent is a molecule comprising one or more antigen-binding moieties. An anti-CCR8 binding agent comprises an anti-CCR8 antibody, an antigen-binding fragment thereof, a binding polypeptide which targets the same, modified derivatives and/or variants thereof.

In some embodiments, anti-CCR8 binding agents provided herein can bind CCR8. Binding affinities for an anti-CCR8 binding agent provided herein to a target such as CCR8 can be determined by measuring, for example, the half maximal effective concentration (EC₅₀) for binding to CCR8-expressing cell lines. EC₅₀, expressed as a molar concentration (e.g., nM), is the concentration of the anti-CCR8 binding agent that achieves half of the maximal binding. In some embodiments, anti-CCR8 binding agents provided herein can bind CCR8, or bind cells expressing CCR8 with an EC₅₀ of about 1 nM or lower, about 0.9 nM or lower, about 0.8 nM or lower, about 0.7 nM or lower, about 0.6 nM or lower, about 0.5 nM or lower, about 0.4 nM or lower, about 0.3 nM or lower, about 0.2 nM or lower, about 0.1 nM or lower, about 0.09 nM or lower, about 0.08 nM or lower, about 0.07 nM or lower, about 0.06 nM or lower, about 0.05 nM or lower, about 0.04 nM or lower, about 0.03 nM or lower, about 0.02 nM or lower, about 0.01 nM or lower, about 0.009 nM or lower, about 0.008 nM or lower, about 0.007 nM or lower, about 0.006 nM or lower, about 0.005 nM or lower, about 0.004 nM or lower, about 0.003 nM or lower, about 0.002 nM or lower, or about 0.001 nM or lower. In some embodiments, anti-CCR8 binding agents provided herein can bind CCR8, or bind cells expressing CCR8 with an EC₅₀ of about 0.80 nM or lower, 0.70 nM or lower, 0.60 nM or lower, 0.50 nM or lower, 0.40 nM or lower, or about 0.30 nM or lower.

An anti-CCR8 binding agent may preferentially bind to CCR8, such as human CCR8, meaning that the antibody or fragment thereof binds CCR8 with greater affinity than it binds to an unrelated control protein and/or binds human CCR8 with greater affinity than it binds to an unrelated control protein. In some embodiments, the unrelated control protein is CCR1. In some embodiments, the unrelated control protein is CCR2. In some embodiments, the unrelated control protein is CCR3. In some embodiments, the unrelated control protein is CCR4. In some embodiments, the unrelated control protein is CCR5. In some embodiments, the unrelated control protein is CCR6. In some embodiments, the unrelated control protein is CCR7. In some embodiments, the unrelated control protein is CCR9.

For example, the antibody or fragment thereof may specifically recognize and bind CCR8 or a portion thereof. In another example, the antibody or fragment thereof may specifically recognize and bind CCR8 or a portion thereof over any one or combination of CCR1-7, and 9 or portion(s) thereof. For example, the antibody or fragment thereof may specifically recognize and bind CCR8 or a portion thereof over CCR1 or a portion thereof. For example, the antibody or fragment thereof may specifically recognize and bind CCR8 or a portion thereof over CCR2 or a portion thereof. For example, the antibody or fragment thereof may specifically recognize and bind CCR8 or a portion thereof over CCR3 or a portion thereof. For example, the antibody or fragment thereof may specifically recognize and bind CCR8 or a portion thereof over CCR4 or a portion thereof. For example, the antibody or fragment thereof may specifically recognize and bind CCR8 or a portion thereof over CCR5 or a portion thereof. For example, the antibody or fragment thereof may specifically recognize and bind CCR8 or a portion thereof over CCR6 or a portion thereof. For example, the antibody or fragment thereof may specifically recognize and bind CCR8 or a portion thereof over CCR7 or a portion thereof. For example, the antibody or fragment thereof may specifically recognize and bind CCR8 or a portion thereof over CCR9 or a portion thereof.

Binding affinities for an anti-CCR8 binding agent provided herein to a target such as CCR8 can be determined by measuring, for example, the dissociation constant (K_{D}). K_{D} as used herein has molar units (M) and corresponds to the concentration of the binding agent at which half of the target proteins are occupied at equilibrium. The smaller the dissociation constant is, the higher is the affinity between the binder or antibody and its target. In some embodiments, anti-CCR8 binding agents provided herein can specifically bind CCR8, or can specifically bind cells expressing CCR8 with a K_{D} of at least about 10⁻⁷ M, at least about 10⁻⁸ M, at least about 10⁻⁹ M, at least about 10⁻¹⁰ M, at least about 10⁻¹¹ M, or less. The KD values can be determined by means of surface plasmon resonance spectroscopy. Where assay conditions were found to influence the determined KD, the assay setup with the least standard deviation shall be used. In some embodiments, anti-CCR8 binding agents can specifically bind to CCR8 with an affinity that is at least 5, 10, 15, 20, 25, 50, 100, 250, 500, 1000, or 10,000 times greater than the affinity for an unrelated control protein (e.g., hen egg white lysozyme). In some embodiments, the anti-CCR8 agent may bind CCR8 substantially exclusively (e.g., is able to distinguish CCR8 from other known polypeptides (e.g., CCR1, CCR2, CCR3, CCR4, CCR5, CCR6, CCR7, CCR9, or any combination thereof), for example, by virtue of measurable differences in binding affinity). In some embodiments, a CCR8 binding agent (e.g., an antibody) may react with CCR8 sequences other than human CCR8 sequences (e.g., rabbit CCR8 sequences).

Binding specificity or selectivity can be determined by measuring, for example, a binding signal in an immunohistochemistry (IHC) assay. In some embodiments, a binding signal will be about 1.5, 2, 2.5, 3, 3.5, 4, 4.5, 5, 5.5, 6, 6.5, 7, 7.5, 8, 8.5, 9, 9.5, or about 10 times more than a background signal or noise. See, e.g., Fundamental Immunology 332-36 (Paul ed., 2d ed. 1989) for a discussion regarding binding specificity.

In some embodiments, anti-CCR8 binding agents provided herein does not bind the same or essentially the same epitope as a reference anti-CCR8 binding agent. In some embodiments, anti-CCR8 binding agents provided herein do not recognize identical, overlapping or adjacent epitopes in a three-dimensional space as a reference anti-CCR8 binding agent. In other words, the anti-CCR8 binding agents provided herein do not compete with reference anti-CCR8 binding agents, such as a therapeutic anti-CCR8 agent described herein.

The most widely used and rapid methods for determining whether two binding agents bind to identical, overlapping or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent or enzyme labels. In other assays, fresh frozen tissue or formalin-fixed paraffin-embedded (FFPE) tissue known to express CCR8 is incubated with an anti-CCR8 binding agent provided herein and a reference anti-CCR8 binding agent, e.g., an antibody targeting an extracellular domain of CCR8, using staining buffers to determine if dual-binding has occurred where both colorimetric stains can be detected. Thus, another method to determine whether two binding agents bind to non-identical, non-overlapping or non-adjacent epitopes in a three-dimensional space are immunohistochemistry (IHC) assays. In some embodiments, a reference anti-CCR8 binding agent is a second anti-CCR8 binding agent as provided herein, including a therapeutic anti-CCR8 agent described herein.

In some embodiments, anti-CCR8 binding agents does not cross-compete with a reference anti-CCR8 binding agent. Cross-competition occurs when two binding agents are provided that both bind the same target, wherein binding of a first binding agent to the target sterically hinders the binding of a second binding agent to the target. Thus, the first binding agent reduces the binding of the second binding agent to the target. Accordingly, consistent with the present disclosure are anti-CCR8 binding agents which do not reduce the binding of a second binding agent to CCR8. Cross-competing binding agents can be readily identified based on their ability to detectably compete in standard antigen binding assays, including BIACORE® analysis, ELISA assays or flow cytometry, using either recombinant antigen molecules or cell-surface expressed antigen molecules, and/or IHC assays, using tissue containing cells that express CCR8 and colorimetric stains.

Accordingly, in some embodiments, and as described in further detail herein, anti-CCR8 agents are useful in systems for detecting CCR8, related systems of diagnosing, and compositions, methods, kits and devices thereof. In some embodiments, systems, compositions, methods, kits and devices can further comprise a second anti-CCR8 agent. In some embodiments, second anti-CCR8 agents are therapeutic anti-CCR8 agents. Such second anti-CCR8 agents, systems, compositions, methods, kits and devices are further described herein.

In some embodiments, anti-CCR8 binding agents provided herein can bind immune effector cells or bind effector proteins (e.g., effector receptors on immune effector cells). Immune effector cells and/or effector proteins can be inherently existing in the immune system of the subject being administered an anti-CCR8 agent, or can be autologous or allogeneic.

In some embodiments, anti-CCR8 binding agents provided herein can cross-link immune effector cells or effector proteins and CCR8 expressing cells. In some embodiments, cell killing is induced by binding of an anti-CCR8 agent provided herein to a CCR8 expressing cell and to an immune effector cell and/or to an effector protein (e.g., cross-linking), which will then mediate the killing of the CCR8 expressing cell.

In some embodiments, anti-CCR8 binding agents provided herein can bind immune effector cells with an EC₅₀ of about 1 nM or lower, about 0.9 nM or lower, about 0.8 nM or lower, about 0.7 nM or lower, about 0.6 nM or lower, about 0.5 nM or lower, about 0.4 nM or lower, about 0.3 nM or lower, about 0.2 nM or lower, about 0.1 nM or lower, about 0.09 nM or lower, about 0.08 nM or lower, about 0.07 nM or lower, about 0.06 nM or lower, about 0.05 nM or lower, about 0.04 nM or lower, about 0.03 nM or lower, about 0.02 nM or lower, about 0.01 nM or lower, about 0.009 nM or lower, about 0.008 nM or lower, about 0.007 nM or lower, about 0.006 nM or lower, about 0.005 nM or lower, about 0.004 nM or lower, about 0.003 nM or lower, about 0.002 nM or lower, or about 0.001 nM or lower. In some embodiments, anti-CCR8 binding agents provided herein can bind CCR8, or bind cells expressing CCR8 with an EC₅₀ of about 0.80 nM or lower, 0.70 nM or lower, 0.60 nM or lower, 0.50 nM or lower, 0.40 nM or lower, or about 0.30 nM or lower, 0.20 nM or lower, 0.10 nM or lower, 0.09 nM or lower, 0.08 nM or lower, 0.07 nM or lower, 0.06 nM or lower, 0.05 nM or lower, 0.04 nM or lower, or 0.03 nM or lower. In some embodiments, anti-CCR8 binding agents provided herein can bind immune effector cells with an EC₅₀ of about 0.1 nM or lower, about 0.09 nM or lower, about 0.08 nM or lower, about 0.07 nM or lower, about 0.06 nM or lower, about 0.05 nM or lower, about 0.04 nM or lower, about 0.03 nM or lower, about 0.02 nM or lower, or about 0.01 nM or lower.

In some embodiments, the immune effector cell is a natural killer cell, macrophage, monocyte, or eosinophil and the effector receptor is the Fcγ receptor (FcγR or FCGR). In certain embodiments, upon cross-linking the immune effector cell and the CCR8 expressing cell, the immune effector cell is induced to secrete substances that mediate cell death, such as lyase, perforin, granzyme, TNF. Therefore, in such embodiments, anti-CCR8 agents provided herein can induce ADCC activity.

In some embodiments, the immune effector cell is a monocyte, macrophage, neutrophil or a dendritic cell and the effector receptor is FcγRIIa, FcγRI, or FcγRIIIa. In certain embodiments, upon cross-linking the immune effector cell and the CCR8 expressing cell, the immune effector cell is induced initiate phagocytosis. Therefore, in such embodiments, anti-CCR8 agents provided herein can induce ADCP activity.

In some embodiments, the immune effector cell is a monocyte, macrophage, neutrophil or a dendritic cell and the effector receptor is FcγRIIa, FcγRI, or FcγRIIIa. In certain embodiments, upon cross-linking the immune effector cell and the CCR8 expressing cell, the immune effector cell is induced initiate phagocytosis. Therefore, in such embodiments, anti-CCR8 agents provided herein can induce ADCP activity.

In some embodiments, the effector protein is C1q. In certain embodiments, upon cross-linking the effector protein and the CCR8 expressing cell, the immune effector cell is induced initiate the complement cascade, resulting in the formation of a membrane attack complex (MAC) and eliciting cell lysis. Therefore, in such embodiments, anti-CCR8 agents provided herein can induce CDC activity.

Accordingly, there are multiple mechanisms of action encompassed by the present disclosure to induce killing of cells in a diseased microenvironment, wherein any one or combination of mechanism of actions can be effected by the disclosure provided herein. A first mechanism of action comprises inducing antibody-dependent cellular cytotoxicity (ADCC). A second mechanism of action comprises inducing antibody-dependent cellular phagocytosis (ADCP). A third mechanism of action comprises inducing complement-dependent cytotoxicity (CDC). A fourth mechanism comprises delivering a therapeutic agent to the diseased microenvironment, such as targeting CCR8 with the anti-CCR8 binding agent in the form of an antibody drug conjugate. Further examples of each of the foregoing are further described herein.

In some embodiments, an anti-CCR8 agent provided herein can induce cell killing and therefore mediate depletion of CCR8 expression and/or depletion of the amount of CCR8 expressing cells. In some embodiments, the anti-CCR8 agent can mediate depletion of CCR8 expression and/or CCR8 expressing cells by less than 5%, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or by more than 95% relative to the amount of CCR8 expression or the amount of CCR8 expressing cells relative to a baseline control.

In some embodiments, an anti-CCR8 agent provided herein can induce ADCC. In some embodiments, an anti-CCR8 agent provided herein can elicit lysis of CCR8 expressing cells in the presence of immune cells (e.g., NK effector cells) by inducing ADCC. In some embodiments, the ADCC induction results in the lysis of at least 2%, 5%, 10%, 15%, 20%, 25%, 30%, 35%, 40%, 45%, 50%, 55%, 60%, 65%, 70%, 75%, 80%, 85%, 90%, 95% or 99% of the CCR8 expressing cells. In some embodiments, an anti-CCR8 agent provided herein can induce ADCC. In some embodiments, an anti-CCR8 agent provided herein exhibits enhanced ADCC activity relative to a control. In some embodiments, an anti-CCR8 agent provided herein exhibits a 2%, 5%, 10%, 15%, 20%, 25%, 30%, 35%, 40%, 45%, 50%, 55%, 60%, 65%, 70%, 75%, 80%, 85%, 90%, 95% or 99% enhanced ADCC activity relative to a control. In some embodiments, a control can be a WT control or a parental control.

In some embodiments, an anti-CCR8 agent provided herein can induce ADCP. In some embodiments, an anti-CCR8 agent provided herein can elicit phagocytosis of CCR8 expressing cells in the presence of immune cells (e.g., macrophages) by inducing ADCP. In some embodiments, the ADCP induction results in the phagocytosis of at least 2%, 5%, 10%, 15%, 20%, 25%, 30%, 35%, 40%, 45%, 50%, 55%, 60%, 65%, 70%, 75%, 80%, 85%, 90%, 95% or 99% of the CCR8 expressing cells.

In some embodiments, an anti-CCR8 agent provided herein can induce CDC. In some embodiments, an anti-CCR8 agent provided herein can elicit formation of a membrane attack complex and lysis of CCR8 expressing cells in the presence of effector proteins (e.g., C1q) by inducing CDC. In some embodiments, the CDC induction results in the lysis of at least 2%, 5%, 10%, 15%, 20%, 25%, 30%, 35%, 40%, 45%, 50%, 55%, 60%, 65%, 70%, 75%, 80%, 85%, 90%, 95% or 99% of the CCR8 expressing cells.

In some embodiments, assays, systems, and devices are provided herein for identifying anti-CCR8 binding agents having one or more biological activity as described herein. Biological activity may include, e.g., antibody-dependent cellular cytotoxicity (ADCC), ADCC against Tregs, antibody-dependent cellular phagocytosis (ADCP), ADCP depletion of Tregs, complement dependent cytotoxicity (CDC), and/or CDC depletion of Tregs. Anti-CCR8 binding agents having such biological activity in vivo and/or in vitro are also provided.

In some embodiments, cytotoxicity assays, systems, and devices are provided herein to confirm the reduction/depletion induced by ADCP, CDC and/or ADCC activities. Such cytotoxicity assays, systems, and devices can be in vitro and/or in vivo. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)); or as described herein. Alternatively, non-radioactive assays methods may be employed (see, for example, ACTI™ non-radioactive cytotoxicity assay for flow cytometry (CellTechnology, Inc. Mountain View, Calif.; and CytoTox 96© non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

In some embodiments, the CCR8 expressing cell is an immunosuppressive Treg cell. In some embodiments, the CCR8 expressing cell is tumor resident Treg cell. In some embodiments, the CCR8 expressing cell is tumor infiltrating Treg cell. In some embodiments, the CCR8 expressing cell is a FOXP3^{hi} Treg cell. In some embodiments, the CCR8 expressing cell is a CD4+FOXP3^{hi} Treg cell. In some embodiments, the CCR8 expressing cell is a tumor infiltrating CD4+FOXP3^{hi} Treg cell. In some embodiments, the CCR8 expressing cell is not a peripheral Treg cell, a peripheral Teff cell, a FOXP3^{mid} CD8+ Teff cell, a FOXP3^{mid} CD4+ Teff cell, FOXP3^{neg} CD8+ Teff cell, a FOXP3^{neg} CD4+ Teff cell, a tumor-infiltrating CD4+ T cell, a tumor-infiltrating CD8+ T cell, or any combination thereof.

In some embodiments, the anti-CCR8 agent can mediate depletion of CCR8 expression and/or depletion of the amount of CCR8 expressing cells in a diseased microenvironment. Accordingly, in some embodiments, the anti-CCR8 agents provided herein can mediate activity in a diseased microenvironment. In some embodiments, a diseased microenvironment is a microenvironment associated with CCR8 expression, including aberrant, upregulated, and/or selective CCR8 expression. In certain embodiments, CCR8 expression is upregulated and/or selectively expressed on immunosuppressive Treg cells, tumor resident Treg cells, tumor infiltrating Treg cells, FOXP3hi Treg cells, CD4+FOXP3hi Treg cell, tumor infiltrating CD4+FOXP3hi Treg cells, or any combination thereof. Accordingly, in some embodiments, a diseased microenvironment provided herein is one that is characterized with CCR8 expression on immunosuppressive Treg cells, tumor resident Treg cells, tumor infiltrating Treg cells, FOXP3hi Treg cells, CD4+FOXP3hi Treg cell, tumor infiltrating CD4+FOXP3hi Treg cells, or any combination thereof.

In some embodiments, mediating activity in a diseased microenvironment comprises mediating the increase of an immune cell population, and specifically an immune effector cell population. In some embodiments, anti-CCR8 agent provided herein can mediate the increase of immune effector cell population by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 300%, about 350%, about 400%, or more relative to a baseline control. In some embodiments, the immune effector cell population comprise CD45+ cells (e.g., intra-tumoral CD45+ cells), CD8+ T cells (e.g., intra-tumoral CD8+ T cells), CD4+ T cells (e.g., intra-tumoral CD4+ T cells), macrophages, such as M1 macrophages or M2 macrophages, (e.g., intra-tumoral macrophages, intratumoral M1 macrophages, intratumoral M2 macrophages), NK cells (e.g., intra-tumoral NK cells), B cells, (e.g., intra-tumoral B cells), Dendritic cells (e.g., intra-tumoral Dendritic cells), Gamma delta T cells (e.g., intra-tumoral Gamma delta T cells), iNKT cells (e.g., intra-tumoral iNKT cells), or any combination thereof.

In some embodiments, the CCR8 binding agents (e.g., antibodies) described herein compete for the binding to CCR8, such as human CCR8, with a CCR8 binding agent (e.g., an antibody) that comprises a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 depicted in TABLES 2-4. Accordingly, in some embodiments, a CCR8 binding agent (e.g., an antibody) described herein competes for the binding to CCR8, such as human CCR8, with a CCR8 binding agent (e.g., an antibody) that comprises one, two, and/or three VH CDRs and/or one, two, and/or three VL CDRs from the antibody designated AB-1 as shown in TABLE 2. In some embodiments, a CCR8 binding agent (e.g., an antibody) described herein competes for the binding to CCR8, such as human CCR8, with a CCR8 binding agent (e.g., an antibody) that comprises one, two, and/or three VH CDRs and one, two, and/or three VL CDRs from the antibody designated AB-1 as shown in TABLE 2. In some embodiments, a CCR8 binding agent (e.g., an antibody) described herein competes for the binding to CCR8, such as human CCR8, with a CCR8 binding agent (e.g., an antibody) that comprises a VH region and VL region from the antibody designated AB-1 as shown in TABLE 3.

In some embodiments, the CCR8 binding agents (e.g., antibodies) described herein comprise a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 depicted in TABLES 2-3. Accordingly, in some embodiments, a CCR8 binding agent (e.g., an antibody) described herein comprises one, two, and/or three heavy chain CDRs and/or one, two, and/or three light chain CDRs from the antibody designated AB-1, as shown in TABLE 2. In some embodiments, a CCR8 binding agent (e.g., an antibody) described herein comprises one, two, and/or three heavy chain CDRs and one, two, and/or three light chain CDRs from the antibody designated AB-1, as shown in TABLE 2.

In some embodiments, an anti-CCR8 binding agent comprises a heavy chain variable region and a light chain variable region. In some embodiments, an anti-CCR8 binding agent comprises at least one heavy chain comprising a heavy chain variable region and at least a portion of a heavy chain constant region, and at least one light chain comprising a light chain variable region and at least a portion of a light chain constant region. In some embodiments, an anti-CCR8 binding agent comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other binding agent (e.g., antibody) that comprises, for example, a single polypeptide chain comprising all six CDRs (three heavy chain CDRs and three light chain CDRs) is considered to have a heavy chain and a light chain. In some embodiments, the heavy chain is the region of the anti-CCR8 binding agent that comprises the three heavy chain CDRs. In some embodiments, the light chain is the region of the anti-CCR8 binding agent that comprises the three light chain CDRs.

Accordingly, in some embodiments, a CCR8 binding agent (e.g., an antibody) comprises a VH region, which comprises VH CDR1, VH CDR2, and/or VH CDR3, and a VL region, which comprises VL CDR1, VL CDR2, and/or VL CDR3, of any one of the binding agents described herein (see, e.g., TABLE 2). In some embodiments, a CCR8 binding agent (e.g., an antibody) described herein is multispecific (e.g., bispecific) and comprises a first binding domain that comprises one, two, and/or three heavy chain CDRs (e.g., VH CDR1, VH CDR2, and VH CDR3) and/or one, two, and/or three light chain CDRs (e.g., VL CDR1, VL CDR2, and VL CDR3) from TABLE 2 and a second binding domain that comprises one, two, and/or three heavy chain CDRs (e.g., VH CDR1, VH CDR2, and VH CDR3) and/or one, two, and/or three light chain CDRs (e.g., VL CDR1, VL CDR2, and VL CDR3) from a binding agent that binds to a second target antigen that is not CCR8.

In some embodiments, a CCR8 binding agent (e.g., an antibody) described herein comprises one, two, and/or three heavy chain CDRs and/or one, two, and/or three light chain CDRs from the antibody designated AB-1, as shown in TABLE 2.

In some embodiments, the anti-CCR8 binding agent comprises six CDRs including (a) HCDR1 comprising the amino acid sequence of SEQ ID NO: 5; (b) HCDR2 comprising the amino acid sequence of SEQ ID NO: 6; (c) HCDR3 comprising the amino acid sequence of SEQ ID NO: 7; (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10.

In some embodiments, the CDRs of anti-CCR8 binding agents including a human CCR8 binding agent, can be determined according to any suitable numbering system. Examples of numbering systems used herein are Kabat, IMGT, North, CCG, Honegger, and Chothia, however, a person of ordinary skill in the art would readily understand that the position of one or more CDRs along the VH (e.g., CDR1, CDR2, or CDR3) and/or VL (e.g., CDR1, CDR2, or CDR3) region of a CCR8 binding agent (e.g., an antibody), including a human CCR8 binding agent, described herein may vary by one, two, three, four, five, or six amino acid positions so long as binding to CCR8 (e.g., human CCR8) is maintained (e.g., substantially maintained, for example, at least 50%, at least 60%, at least 70%, at least 80%, at least 90%, at least 95%). For example, in some embodiments, the position defining a CDR as described in TABLE 2 may vary by shifting the N-terminal and/or C-terminal boundary of the CDR by one, two, three, four, five, or six amino acids, relative to the current CDR position, so long as binding to CCR8 (e.g., human CCR8) is maintained (e.g., substantially maintained, for example, at least 50%, at least 60%, at least 70%, at least 80%, at least 90%, at least 95%). In other embodiments, the length of one or more CDRs along the VH (e.g., CDR1, CDR2, or CDR3) and/or VL (e.g., CDR1, CDR2, or CDR3) region of a CCR8 binding agent (e.g., an antibody), including a human CCR8 binding agent, described herein may vary (e.g., be shorter or longer) by one, two, three, four, five, or more amino acids, so long as binding to CCR8 (e.g., human CCR8) is maintained (e.g., substantially maintained, for example, at least 50%, at least 60%, at least 70%, at least 80%, at least 90%, at least 95%). For example, in some embodiments, a VH and/or VL CDR1, CDR2, and/or CDR3 described herein may be one, two, three, four, five or more amino acids shorter than one or more of the CDRs described by any one of the sequences set forth in TABLE 2, so long as binding to CCR8 (e.g., human CCR8) is maintained (e.g., substantially maintained, for example, at least 50%, at least 60%, at least 70%, at least 80%, at least 90%, at least 95%). In other embodiments, a VH and/or VL CDR1, CDR2, and/or CDR3 described herein may be one, two, three, four, five or more amino acids longer than one or more of the CDRs described by any one of the sequences set forth in TABLE 2, so long as binding to CCR8 (e.g., human CCR8) is maintained (e.g., substantially maintained, for example, at least 50%, at least 60%, at least 70%, at least 80%, at least 90%, at least 95%). In other embodiments, the amino terminus of a VH and/or VL CDR1, CDR2, and/or CDR3 described herein may be extended by one, two, three, four, five or more amino acids compared to one or more of the CDRs described by any one of the sequences set forth in TABLE 2, so long as binding to CCR8 (e.g., human CCR8) is maintained (e.g., substantially maintained, for example, at least 50%, at least 60%, at least 70%, at least 80%, at least 90%, at least 95%). In other embodiments, the carboxy terminus of a VH and/or VL CDR1, CDR2, and/or CDR3 described herein may be extended by one, two, three, four, five or more amino acids compared to one or more of the CDRs described by any one of the sequences set forth in TABLE 2, so long as binding to CCR8 (e.g., human CCR8) is maintained (e.g., substantially maintained, for example, at least 50%, at least 60%, at least 70%, at least 80%, at least 90%, at least 95%). In other embodiments, the amino terminus of a VH and/or VL CDR1, CDR2, and/or CDR3 described herein may be shortened by one, two, three, four, five or more amino acids compared to one or more of the CDRs described by any one of the sequences set forth in TABLE 2, so long as binding to CCR8 (e.g., human CCR8) is maintained (e.g., substantially maintained, for example, at least 50%, at least 60%, at least 70%, at least 80%, at least 90%, at least 95%). In some embodiments, the carboxy terminus of a VH and/or VL CDR1, CDR2, and/or CDR3 described herein may be shortened by one, two, three, four, five or more amino acids compared to one or more of the CDRs described by any one of the sequences set forth in TABLE 2, so long as binding to CCR8 (e.g., human CCR8) is maintained (e.g., substantially maintained, for example, at least 50%, at least 60%, at least 70%, at least 80%, at least 90%, at least 95%). Any method known in the art can be used to ascertain whether binding to CCR8 (e.g., human CCR8) is maintained, for example, the binding assays and conditions described in the “Examples” section described herein.

In some embodiments, CCR8 binding agents (e.g., antibodies, such as bispecific antibodies), including human CCR8 binding agents, described herein comprise a VH region or VH domain. In other embodiments, CCR8 binding agents (e.g., antibodies, such as bispecific antibodies), including human CCR8 binding agents, described herein comprise a VL region or VL domain. In some embodiments, CCR8 binding agents (e.g., antibodies, such as bispecific antibodies), including human CCR8 binding agents, described herein have a combination of (i) a VH domain or VH region; and/or (ii) a VL domain or VL region.

In some embodiments, a CCR8 binding agent (e.g., an antibody) described herein comprises a heavy chain variable domain (VH) sequence and/or a light chain variable domain (VL) sequence having at least 90%, 91%, 92%, 93%, 94%, 95%, 96%, 97%, 98%, 99%, or 100% sequence identity to the amino acid sequences from the antibody designated AB-1, as shown in TABLE 3.

In some embodiments, a CCR8 binding agent (e.g., an antibody) described herein comprises a heavy chain variable domain (VH) sequence having at least 90%, 91%, 92%, 93%, 94%, 95%, 96%, 97%, 98%, 99%, or 100% sequence identity to the VH amino acid sequence from the antibody designated AB-1, as shown in TABLE 3. In some embodiments, an anti-CCR8 binding agent comprises a VH sequence having at least 90%, 91%, 92%, 93%, 94%, 95%, 96%, 97%, 98%, 99%, or 100% sequence identity to the amino acid sequence of SEQ ID NO: 11. In some embodiments, a VH sequence having at least 90%, 91%, 92%, 93%, 94%, 95%, 96%, 97%, 98%, or 99% identity contains amino acid modifications, such as substitutions (for example, conservative substitutions), insertions, or deletions relative to the reference sequence, but an anti-CCR8 binding agent comprising that sequence retains the ability to bind to CCR8. In some embodiments, a total of 1 to 10 amino acids (e.g., 1, 2, 3, 4, 5, 6, 7, 8, 9, or 10 amino acids) have been modified, e.g., substituted, inserted and/or deleted in SEQ ID NO: 11. In some embodiments, modifications, e.g., substitutions, insertions, or deletions, occur in regions outside the CDRs (that is, in the FRs). Optionally, the anti-CCR8 binding agent comprises the VH sequence in SEQ ID NO: 11, including post-translational modifications of that sequence. In some embodiments, an anti-CCR8 binding agent as described herein comprises (a) HCDR1 comprising the amino acid sequence of SEQ ID NO: 5; (b) HCDR2 comprising the amino acid sequence of SEQ ID NO: 6; and/or (c) HCDR3 comprising the amino acid sequence of SEQ ID NO: 7.

In some embodiments, a CCR8 binding agent (e.g., an antibody) described herein comprises a light chain variable domain (VL) sequence having at least 90%, 91%, 92%, 93%, 94%, 95%, 96%, 97%, 98%, 99%, or 100% sequence identity to the VL amino acid sequence from the antibody designated AB-1, as shown in TABLE 3. In some embodiments, an anti-CCR8 binding agent comprises a VL sequence having at least 90%, 91%, 92%, 93%, 94%, 95%, 96%, 97%, 98%, 99%, or 100% sequence identity to the amino acid sequence of SEQ ID NO: 12. In some embodiments, a VL sequence having at least 90%, 91%, 92%, 93%, 94%, 95%, 96%, 97%, 98%, or 99% identity contains amino acid modifications, such as substitutions (for example, conservative substitutions), insertions, or deletions relative to the reference sequence, but an anti-CCR8 binding agent comprising that sequence retains the ability to bind to CCR8. In some embodiments, a total of 1 to 10 amino acids (e.g., 1, 2, 3, 4, 5, 6, 7, 8, 9, or 10 amino acids) have been modified, e.g., substituted, inserted and/or deleted in SEQ ID NO: 12. In some embodiments, modifications, e.g., substitutions, insertions, or deletions, occur in regions outside the CDRs (that is, in the FRs). Optionally, the anti-CCR8 binding agent comprises the VL sequence in SEQ ID NO: 12, including post-translational modifications of that sequence. In some embodiments, an anti-CCR8 binding agent as described herein comprises (a) LCDR1 comprising the amino acid sequence of SEQ ID NO: 8; (b) LCDR2 comprising the amino acid sequence of SEQ ID NO: 9; and/or (c) LCDR3 comprising the amino acid sequence of SEQ ID NO: 10.

In some embodiments, a CCR8 binding agent (e.g., an antibody) described herein comprises a heavy chain (HC) and/or a light chain (LC) sequence having at least 90%, 91%, 92%, 93%, 94%, 95%, 96%, 97%, 98%, 99%, or 100% sequence identity to the amino acid sequences from the antibody designated AB-1, as shown in TABLE 4.

In some embodiments, a CCR8 binding agent (e.g., an antibody) described herein comprises a heavy chain (HC) having at least 90%, 91%, 92%, 93%, 94%, 95%, 96%, 97%, 98%, 99%, or 100% sequence identity to the amino acid sequence from the antibody designated AB-1, as shown in TABLE 4. In some embodiments, an anti-CCR8 binding agent is provided, wherein the antibody comprises a HC having at least 90%, 91%, 92%, 93%, 94%, 95%, 96%, 97%, 98%, 99%, or 100% sequence identity to the amino acid sequence of SEQ ID NO: 13. Optionally, the anti-CCR8 binding agent comprises the HC sequence in SEQ ID NO: 13, including post-translational modifications.

In some embodiments, a CCR8 binding agent (e.g., an antibody) described herein comprises a light chain (LC) having at least 90%, 91%, 92%, 93%, 94%, 95%, 96%, 97%, 98%, 99%, or 100% sequence identity to the amino acid sequence from the antibody designated AB-1, as shown in TABLE 4. In some embodiments, an anti-CCR8 binding agent is provided, wherein the antibody comprises a LC having at least 90%, 91%, 92%, 93%, 94%, 95%, 96%, 97%, 98%, 99%, or 100% sequence identity to the amino acid sequence of SEQ ID NO: 14. Optionally, the anti-CCR8 binding agent comprises the LC sequence in SEQ ID NO: 14, including post-translational modifications.

Anti-CCR8 agents provided herein can be antibodies, fragments thereof or binding polypeptides targeting the same. It is understood that referring to an antibody anti-CCR8 agent, reference is also made to fragments thereof, and binding polypeptides comprising the same, and vice versa. Accordingly, provided herein are anti-CCR8 antibodies and fragments thereof.

### A. Antibodies

In some embodiments, an anti-CCR8 agent provided herein is an anti-CCR8 antibody or a functional fragment thereof. Accordingly, provided herein are anti-CCR8 antibodies, fragments thereof, or uses thereof. In some embodiments, provided herein is an isolated anti-CCR8 antibody. In some embodiments, provided herein is an isolated anti-CCR8 antibody fragment. In some embodiments, an anti-CCR8 antibody provided herein can be used in therapeutic and/or diagnostic methods, such as methods of detection of CCR8 in a sample, methods of treatment, methods of diagnosis, and/or methods of prognosis. Diagnostic and therapeutic methods are further described herein. It is understood that when referencing an antibody herein, reference is also being made to a functional fragment thereof (e.g., a single-chain antibody, an isolated antibody hypervariable domain, a binding fragment thereof) and modified variants thereof, including a derivative thereof (e.g., an antibody conjugated with a substrate or with the protein or ligand of a protein-ligand pair).

In some embodiments, antibodies described herein include immunoglobulin molecules and immunologically active portions of immunoglobulin molecules, including molecules that contain one or more antigen binding sites that bind to a CCR8 antigen.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In some embodiments, antibodies described herein are IgG antibodies (e.g., human IgG), or a class (e.g., human IgG1, IgG2, IgG3 or IgG4) or subclass thereof.

In some embodiments, an antibody is a 4-chain antibody unit comprising two heavy (H) chain/light (L) chain pairs, wherein the amino acid sequences of the H chains are identical and the amino acid sequences of the L chains are identical. In some embodiments, the H and L chains comprise constant regions, for example, human constant regions. In some embodiments, the L chain constant region of such antibodies is a kappa or lambda light chain constant region, for example, a human kappa or lambda light chain constant region. In some embodiments, the H chain constant region of such antibodies comprise a gamma heavy chain constant region, for example, a human gamma heavy chain constant region. In some embodiments, such antibodies comprise IgG constant regions, for example, human IgG constant regions (e.g., IgG1, IgG2, IgG3, and/or IgG4 constant regions).

Antibodies and fragments thereof described herein include, but are not limited to, synthetic antibodies, monoclonal antibodies, recombinantly produced antibodies, multispecific antibodies (e.g., including bispecific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, Fabs-in-tandem-lg (FIT-lg); DVD-lg; hybrid hybridoma (quadroma or tetradoma); anticalin platform (Pieris); diabodies; single chain diabodies; tandem single chain Fv fragments; TandAbs, Trispecific Abs (Affimed); Darts dual affinity retargeting (Macrogenics); Bispecific Xmabs (Xencor); Bispecific T cell engagers (BiTE; Amgen; 55 kDa); Triplebodies; Tribody=Fab-scFv Fusion Protein multifunctional recombinant antibody derivates (CreativeBiolabs); Duobody platform (Genmab); dock and lock platform; knobs-into-holes (KIH) platform; humanized bispecific IgG antibody (REGN1979) (Regeneron); Mab2 bispecific antibodies (F-Star); DVD-lg=dual variable domain immunoglobulin (AbbVie); kappa-lambda bodies; TBTI=tetravalent bispecific tandem Ig; and CrossMab (Roche), and epitope-binding fragments of any of the above.

### Antibody Fragments

In some embodiments, an antibody provided herein is an antibody fragment. In some embodiments, the antibody fragment is a Fab, Fab′, Fab′-SH, or F(ab′)₂ fragment, and in particular a Fab fragment. Papain digestion of intact antibodies produces two identical antigen-binding fragments, called Fab fragments containing each the heavy- and light-chain variable domains (VH and VL, respectively) and also the constant domain of the light chain (CL) and the first constant domain of the heavy chain (CH1). A Fab fragment thus refers to an antibody fragment comprising a light chain comprising a VL domain and a CL domain, and a heavy chain fragment comprising a VH domain and a CH1 domain. Fab′ fragments differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-binding sites (two Fab fragments) and a part of the Fc region. For discussion of Fab and F(ab′)₂ fragments comprising salvage receptor binding epitope residues and having increased in vivo half-life, see U.S. Pat. No. 5,869,046.

In some embodiments, the antibody fragment is a diabody, a triabody or a tetrabody. Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In some embodiments, the antibody fragment is a single chain Fab fragment. A single chain Fab fragment (or scFab) is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In some embodiments, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues.

In some embodiments, the antibody fragment is single-chain variable fragment (scFv). A single-chain variable fragment (or scFv) is a fusion protein of the variable domains of the heavy (VH) and light chains (VL) of an antibody, connected by a linker. In some embodiments, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In some embodiments, the antibody fragment is a single-domain antibody. Single-domain antibodies are antibody fragments comprising all or a portion of the heavy chain variable domain or all or a portion of the light chain variable domain of an antibody. In some embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

### Chimeric Antibodies

In some embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

Nonlimiting exemplary chimeric antibodies include chimeric antibodies comprising the heavy and/or light chain variable regions of, e.g., AB-1 as disclosed herein. Additional nonlimiting exemplary chimeric antibodies include chimeric antibodies comprising heavy chain CDR1, CDR2, and CDR3, and/or light chain CDR1, CDR2, and CDR3 of an antibody AB-1 as disclosed herein. In some embodiments, the chimeric anti-CCR8 antibody comprises the variable regions described above and binds to CCR8. In some embodiments, the anti-CCR8 antibody comprises the variable regions described above, binds to CCR8 and mediates an immune response in a subject, mediates activity in a diseased microenvironment and/or depletes CCR8 expression and/or CCR8 expressing cells in a subject following administration of the antibody to the subject. Other effector function includes inducing ADCC activity, ADCP activity, and/or CDC activity.

In some embodiments, a chimeric antibody described herein comprises one or more human constant regions. In some embodiments, the human heavy chain constant region is of an isotype selected from IgA, IgG, IgD, and IgE. In some embodiments, the human light chain constant region is of an isotype selected from κ and λ. In some embodiments, a chimeric antibody described herein comprises a human IgG constant region. In some embodiments, a chimeric antibody described herein comprises a human IgG4 heavy chain constant region. In some embodiments, a chimeric antibody described herein comprises a human IgG4 constant region and a human κ light chain.

Whether or not effector function is desirable may depend on the particular method of treatment intended for an antibody. Thus, in some embodiments, when effector function is desirable, a chimeric anti-CCR8 antibody comprising a human IgG1 heavy chain constant region or a human IgG3 heavy chain constant region is selected. In some embodiments, when effector function is not desirable, a chimeric anti-CCR8 antibody comprising a human IgG4 or IgG2 heavy chain constant region is selected. In some embodiments, enhanced effector function is desirable.

### Humanized Antibodies

In some embodiments, an antibody provided herein is a humanized antibody. In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In some embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE@technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

Antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, for example, in Hoogenboom et al. in Methods in Molecular Biology 178: 1-37 (O'Brien et al., ed., Human Press, Totowa, NJ, 2001) and further described, for example, in the McCafferty et al, Nature 348:552-554 (1990); Clackson et al, Nature 352: 624-628 (1991); Marks et al, J. Mol. Biol 222: 581-597(1992); Marks and Bradbury, in Methods in Molecular Biology 248: 161-175 (Lo, ed., Human Press, Totowa, NJ, 2003); Sidhu et al, J. Mol. Biol. 338(2) (2004): 299-310; Lee et al., J. Mol. Biol. 340(5): 1073-1093 (2004); Fellouse, Proc. Natl. Acad. Sci. USA 101(34): 12467-12472 (2004); and Lee et al, (2004) J. Immunol. Methods 284(1-2): 119-132 and PCT publication WO 99/10494.

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12:433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (for example, from human) to provide a single source of antibodies to a wide range of non-self and also self-antigens without any immunization as described by Griffiths et al., EMBO J 12:725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol, 227:381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

In some embodiments, a chimeric human anti-CCR8 antibody is provided, where the antibody comprises the variable region from a human antibody that binds CCR8 and the constant region from a different human antibody. In some embodiments, a chimeric human anti-CCR8 antibody, where the antibody comprises the CDRs from a human antibody that binds CCR8 and a framework from a different human antibody is provided. In some embodiments, the antibody is not a naturally occurring human antibody.

In some embodiments, a human anti-CCR8 antibody comprises one or more human constant regions. In some embodiments, the human heavy chain constant region is of an isotype selected from IgA, IgG, IgD, and IgE. In some embodiments, the human light chain constant region is of an isotype selected from κ and λ. In some embodiments, a human antibody described herein comprises a human IgG constant region. In some embodiments, a human antibody described herein comprises a human IgG4 heavy chain constant region. In some embodiments, a human antibody described herein comprises a human IgG4 constant region and a human κ light chain.

In some embodiments, when effector function is desirable, a human anti-CCR8 antibody comprising a human IgG1 heavy chain constant region or a human IgG3 heavy chain constant region is selected. In some embodiments, when effector function is not desirable, a human anti-CCR8 antibody comprising a human IgG4 or IgG2 heavy chain constant region is selected.

As noted herein, the term “human antibody” denotes the genus of possible sequences for the antibody construct, rather than a source of the antibody.

### Multispecific Antibodies

In some embodiments, an antibody provided herein is a multispecific antibody, e.g., a bispecific antibody. Multispecific antibodies are monoclonal antibodies that have binding specificities for at least two different sites, e.g., different epitopes on different antigens or different epitopes on the same antigen. In certain embodiments, the multispecific antibody has three or more binding specificities. In certain embodiments, one of the binding specificities is for CCR8 and the other specificity is for any other antigen. In certain embodiments, bispecific antibodies may bind to two (or more) different epitopes of CCR8. Multispecific (e.g., bispecific) antibodies may also be used to localize cytotoxic agents or cells to cells which express CCR8. Multispecific antibodies may be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Anti-CCR8 antibodies provided herein can be fused or conjugated to a second molecule. Fusion proteins and antibody conjugates are further described herein.

In some embodiments, antibodies that compete with the anti-CCR8 antibodies provided herein for binding to CCR8 are provided. In some embodiments, antibodies compete with the anti-CCR8 antibodies provided herein for binding to an epitope on CCR8.

In some embodiments, competition assays may be used to identify a monoclonal antibody that competes with an anti-CCR8 antibody described herein (such as AB-1) for binding to CCR8. Competition assays can be used to determine whether two antibodies bind the same epitope by recognizing identical or sterically overlapping epitopes or one antibody competitively inhibits binding of another antibody to the antigen. In some embodiments, such a competing antibody binds to the same epitope that is bound by an antibody described herein. Exemplary competition assays include, but are not limited to, routine assays such as those provided in Harlow and Lane (1988) Antibodies: A Laboratory Manual ch.14 (Cold Spring Harbor Laboratory, Cold Spring Harbor, N.Y.). Detailed exemplary methods for mapping an epitope to which an antibody binds are provided in Morris (1996) “Epitope Mapping Protocols,” in Methods in Molecular Biology vol. 66 (Humana Press, Totowa, N.J.). In some embodiments, two antibodies are said to bind to the same epitope if each blocks binding of the other by 50% or more. In some embodiments, the antibody that competes with an anti-CCR8 antibody described herein is a chimeric, humanized or human antibody. In some embodiments, an antibody that competes with a chimeric, humanized, or human anti-CCR8 antibody as described herein is provided.

In some embodiments, non-competition assays may be used to identify a monoclonal antibody that competes with an anti-CCR8 antibody described herein (such as AB-1) for binding to CCR8. Non-competition assays can be used to determine whether two antibodies bind the different epitopes. In some embodiments, such a non-competing antibody binds to a different epitope that is bound by an antibody described herein. Exemplary non-competition assays include, but are not limited to, routine assays such as those provided in Harlow and Lane (1988) Antibodies: A Laboratory Manual ch.14 (Cold Spring Harbor Laboratory, Cold Spring Harbor, N.Y.). Detailed exemplary methods for mapping an epitope to which an antibody binds are provided in Morris (1996) “Epitope Mapping Protocols,” in Methods in Molecular Biology vol. 66 (Humana Press, Totowa, N.J.). In some embodiments, two antibodies are said to bind to different epitope if each blocks binding of the other by less than 50%. In some embodiments, the antibody that does not competes with an anti-CCR8 antibody described herein is a chimeric, humanized or human antibody. In some embodiments, provided herein a chimeric, humanized, or human anti-CCR8 antibody that does not competes with an anti-CCR8 antibody.

### B. Modification

In some embodiments, anti-CCR8 agents provided herein are engineered with one or more post-translational modifications.

In some embodiments, one or more post-translational modifications comprises one or more amino acid alterations, one or more chemical alterations, one or more conjugation or fusion to one or more second agents, one or more linkers or any combinations thereof.

### Amino Acid Alterations

In some embodiments, one or more modifications comprise one or more amino acid alterations. In some embodiments, anti-CCR8 agents provided herein are engineered with one, 2, 3, 4, 5 or more amino acid alterations. Amino acid alterations comprise one or more amino acid substitutions, deletions, and/or insertions. In some embodiments, the one or more amino acid substitutions comprises conservative substitutions or non-conservative substitutions. With respect to polypeptides that are CCR8 binding agents (e.g., antibodies, fragments, and/or binding polypeptides), such as human CCR8 binding agents, conservative amino acid substitutions that include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, in some embodiments, a predicted nonessential amino acid residue in a CCR8 binding agent is replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). In some embodiments, the conservative amino acid alterations described herein modify the amino acid sequences of the CCR8 binding agents (e.g., antibodies), including human CCR8 binding agents, by 50%, or 55%, or 60%, or 65%, or 70%, or 75%, or 80%, or 85%, or 90%, or 95%, or 98%, or 99%. In some embodiments, the nucleotide and amino acid substitutions refer to at most 1, 2, 3, 4, 5, or 6 amino acid substitutions to the CDRs described in TABLE 2. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution.

In some embodiments, antibodies provided here are affinity matured wherein such an antibody comprises one or more amino acid alterations in one or more CDRs compared to a parent antibody which does not possess such alterations, such alterations resulting in an improvement in the affinity of the antibody for antigen. Anti-CCR8 antibodies provided herein can be affinity matured using suitable selection and/or mutagenesis methods. In some embodiments, affinity matured antibodies have an affinity which is 1.5 times or more, 2 times or more, 3 times or more, 4 times or more, 5 times or more, 10 times or more, 20 times or more, or 30 times or more than that of the starting antibody (e.g., a murine or rabbit humanized or human antibody) from which the matured antibody is prepared from.

In some embodiments, anti-CCR8 agents provided herein comprise one or more modifications which modulate the half-life of antibodies in vivo. For example, addressing the interaction of Fc with FcRn allows to modulate the half-life of antibodies in vivo. In some embodiments, abrogating the interaction by e.g. introduction of mutation H435A leads to an extremely short half-life, since the antibody is no longer protected from lysosomal degradation by FcRn recycling. In some embodiments, anti-CCR8 agents provided herein (e.g., an antibody) comprise a modification comprising a H435A substitution or has otherwise been engineered for a reduced half-life.

In some embodiments, anti-CCR8 agents described herein comprise one or more modifications which extend the half-life of anti-CCR8 agents provided herein. For example, antibodies comprising “YTE” mutations (M252Y/S254T/T256E) and/or equivalent mutations such as “LS” mutations (M428L/N434S) have been shown to significantly extend the half-life by more efficient recycling from endosomes in both pre-clinical species as well as humans (Dall'Acqua, William F., et al. The Journal of Immunology 169.9: 5171-5180 (2002); Zalevsky, Jonathan, et al “Enhanced antibody half-life improves in vivo activity.” Nature biotechnology 28.2 (2010): 157-159.). Accordingly, in some embodiments, the anti-CCR8 agents provided herein comprise YTE mutations (M252Y/S254T/T256E) and/or equivalent mutations such as LS (M428L/N434S) or has otherwise been engineered for an improved half-life. Suitable Fc engineering approaches for extension of half-life can be found in Haraya, Kenta, Tatsuhiko Tachibana, and Tomoyuki Igawa. Drug metabolism and pharmacokinetics 34.1: 25-41 (2019), and/or Lee, Chang-Han, et al. Nature communications 10.1: 1-11 (2019), both incorporated herein by reference.

In some embodiments, an anti-CRR8 agent provided herein comprise one or more modifications which promote the association of the first and the second subunit of the Fc domain. Such modifications include manipulation of the peptide backbone or the post-translational modifications of an Fc domain subunit that reduces or prevents the association of a polypeptide comprising the Fc domain subunit with an identical polypeptide to form a homodimer. Antibodies comprising an Fc region may or may not comprise a modification promoting the association of the first and the second subunit of the Fc domain. A modification promoting association as used herein includes separate modifications made to each of the two Fc domain subunits desired to associate (e.g. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable. Thus, (hetero)dimerization occurs between a polypeptide comprising the first Fc domain subunit and a polypeptide comprising the second Fc domain subunit, which might be non-identical, e.g. in the sense that further components fused to each of the subunits (e.g. antigen binding moieties) are not the same. In some embodiments the modification promoting association comprises an amino acid alteration in the Fc domain, specifically an amino acid substitution. In a particular embodiment, the modification promoting association comprises a separate amino acid alteration, specifically an amino acid substitution, in each of the two subunits of the Fc domain.

### Chemical Alterations

In some embodiments, anti-CCR8 agents provided herein are modified by one or more chemical alterations comprising glycosylation (e.g., afucosylation), acetylation, pegylation, phosphorylation, sulfation, amidation, derivatization by known protecting/blocking groups, proteolytic cleavage, linkage to a cellular ligand or other protein. Any of numerous chemical modifications may be carried out by known techniques, including, but not limited to, specific chemical cleavage, acetylation, formylation, metabolic synthesis of tunicamycin, etc. In some embodiments, an antibody modified by one or more chemical alterations are referred to herein as a derivatized antibody, or a derivative. Additionally, the derivative may contain one or more non-natural amino acids, e.g., using ambrx technology, see, e.g., Wolfson, Wendy. “Amber codon flashing ambrx augments proteins with unnatural amino acids.” Chemistry & biology 13.10 (2006): 1011-1012.

In some embodiments, anti-CCR8 agents provided herein comprise one or more modifications which alter at least one constant region-mediated biological effector function. For example, in some embodiments, an anti-CCR8 agent may be modified to reduce or enhance at least one constant region-mediated biological effector function relative to the unmodified anti-CCR8 agent, e.g., reduced or improved binding to the Fc receptor (FcγR). FcγR binding may be reduced, e.g. by mutating the immunoglobulin constant region segment of the antibody at particular regions necessary for FcγR interactions (see, e.g., Canfield, Stephen M., and Sherie L. Morrison. The Journal of experimental medicine 173.6: 1483-1491 (1991); and Lund, John, et al. The Journal of Immunology 147.8: 2657-2662 (1991)). FcγR binding may be enhanced, e.g. by afucosylation. Reducing FcγR binding may also reduce other effector functions which rely on FcγR interactions, such as opsonization (e.g., CDC), phagocytosis (e.g., ADCP) and antigen-dependent cellular cytotoxicity (e.g., ADCC)

Accordingly, in some embodiments, antibodies and/or binding polypeptides provided herein are modified such that the oligosaccharides in the Fc region of the antibody do not have any or have reduced fucose sugar units (e.g., afucosylated). Removal of the core fucose from the biantennary complex-type oligosaccharides attached to the Fc can greatly increase ADCC effector function without altering antigen binding or CDC effector function. Several ways are known for reducing or abolishing fucosylation of Fc-containing molecules, e.g., antibodies. These include recombinant expression in certain mammalian cell lines including a FUT8 knockout cell line, variant CHO line Lec13, rat hybridoma cell line YB2/0, a cell line comprising a small interfering RNA specifically against the FUT8 gene, and a cell line co-expressing α-1,4-N-acetylglucosaminyltransferase III and Golgi α-mannosidase II. Alternatively, the Fc-containing molecule may be expressed in a non-mammalian cell such as a plant cell, yeast, or prokaryotic cell, e.g., E. coli. Zinc-finger nucleases are another known method of generating afucosylated antibodies. See e.g., Haryadi et al., Bioengineered 4:2, 90-94 (2013); Ripka et al. Arch. Biochem. Biophys. 249:533-545 (1986); Yamane-Ohnuki et al. Biotech. Bioeng. 87: 614 (2004); Pereira et al. mAbs 10(5): 693-711 (2018).

In some embodiments, an anti-CCR8 agent provided herein may be further modified to contain additional nonproteinaceous moieties that are known in the art and readily available. The moieties suitable for derivatization of the anti-CCR8 agent include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the anti-CCR8 agent to be improved, whether the antibody derivative will be used in a therapy under defined conditions

### Fusion or Conjugation Alterations

In some embodiments, a CCR8 binding agent is linked, fused, or conjugated (directly or indirectly), to one or more moieties, which can provide one or more functions. Such moieties include masking moieties, cleavable moieties, second agents (e.g., a diagnostic agent, a detectable agent, a therapeutic agent, such as a cytolytic agent, a second antibody or an antigen-binding fragment thereof, including constructs of multispecific antibodies, a heterologous protein, an effector cell, such as a chimeric antigen receptor T cell (CAR)), and combinations thereof.

### Masking/Cleavable Moieties

In some embodiments, CCR8 binding agent is linked, fused, or conjugated (directly or indirectly), to a masking moiety and/or cleavable moiety in which one or more of the CCR8 binding domains of the CCR8 binding agent (e.g., antibody) are masked (e.g., via a masking moiety) and/or activatable (e.g., via a cleavable moiety). Technologies for masking of a CCR8 binding agent (e.g., an antibody) are well known in the art, including SAFE body masking technology (see, e.g., US Patent Application Publication No. 2019/0241886) and Probody masking technology (see, e.g., US Patent Application Publication No. 2015/0079088). Such technologies can be used to generate a CCR8 binding agent (e.g., an antibody) that is masked and/or activatable. Such masked and/or activatable CCR8 binding agents (e.g., antibodies) are useful for the preparation of conjugates, including immunoconjugates, antibody-drug conjugates (ADCs), masked ADCs and activatable antibody-drug conjugates (AADCs), comprising any one of the CCR8 binding agents (e.g., antibodies), such as human CCR8 binding agents, of the present disclosure, including those directly or indirectly linked to a second agent with effector function.

In some embodiments, CCR8 binding agent is linked, fused, or conjugated (directly or indirectly), to a second agent. In some embodiments, a second agent comprises a diagnostic agent, a detectable agent, or a therapeutic agent, such as a cytolytic agent, an effector cell, or a heterologous protein.

### Detectable/Diagnostic Agents

In some embodiments, an anti-CCR8 agent provided herein can be linked, fused or conjugated to a detectable agent or a diagnostic agent. Examples of detectable agents include various enzymes, prosthetic groups, fluorescent materials, luminescent materials, bioluminescent materials, radioactive materials, positron emitting metals, nonradioactive paramagnetic metal ions and reactive moieties. The detectable agent can be coupled or conjugated either directly to the antibody or fragment thereof or indirectly, e.g. through a linker known in the art or another moiety, using techniques known in the art. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, 0-galactosidase, acetylcholinesterase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 111In or 99mTc.

Detection of expression of a chemokine receptor or CCR8 generally involves contacting a biological sample (tumor, cells, tissue, or body fluid of an individual) with one or more anti-CCR8 agent provided herein (optionally conjugated to a detectable moiety), and detecting whether or not the sample is positive for CCR8, or whether the sample has altered (e.g., reduced or increased) expression as compared to a control sample. Further detectable agents and diagnostic agents are described further herein, for example as detectable labels and/or diagnostic labels.

### Antibodies, Fragments, and Constructs Thereof

In some embodiments, anti-CCR8 binding agents provided herein are linked, conjugated or fused to a second antibody and/or antigen-binding fragments thereof, or constructs of the same.

In some embodiments, anti-CCR8 binding agents provided herein are linked, conjugated or fused to a second antibody to form an antibody heteroconjugate. In some embodiments, anti-CCR8 binding agents (e.g., anti-CCR8 antibodies) provided herein are linked, conjugated or fused to a second binding moiety generating multispecific antibodies. Multispecific antibodies, such as bispecific antibodies, are monoclonal antibodies that have binding specificities for at least two different targets (e.g., antigens) or two different epitopes on the same target (e.g., a bispecific antibody directed to CCR8 with a first binding domain for a first epitope of a CCR8, and a second binding domain for a second epitope of CCR8). In some embodiments, the multispecific (e.g., bispecific) antibodies can be constructed based on the sequences of the antibodies described herein, for example, the CDR sequences in TABLE 2. In some embodiments, the multispecific antibodies described herein are bispecific antibodies. In some embodiments, bispecific antibodies are mouse, chimeric, human or humanized antibodies. In some embodiments, one of the binding specificities of the multispecific antibody is for CCR8 and the other is for any other target (e.g., antigen). In some embodiments, a multispecific (e.g., bispecific) antibody can comprise more than one target (e.g., antigen) binding domain, in which different binding domains are specific for different targets (e.g., a first binding domain that binds to CCR8 and a second binding domain that binds another target (e.g., antigen), such as an immune checkpoint regulator (e.g., a negative checkpoint regulator). In some embodiments, multispecific (e.g., bispecific) antibody molecules can bind than one (e.g., two or more) epitopes on the same target (e.g., antigen). For example, a second binding moiety can be a moiety derived from any one of a therapeutic anti-CCR8 binding agent as further described herein.

In some embodiments, one of the binding specificities is CCR8 and the other is for one or more of a different chemokine receptor, a cell-surface protein such as cell surface protein expressed on immune cells or a tissue- or cell type-specific antigen, or a cell surface molecule associated with T-cell activation. Exemplary different chemokine receptors include CCR1, CCR2, CCR3, CCR4, CCR5, CCR6, CCR7, CCR9, CCR10, CXCR1, CXCR2, CXCR3, CXCR4, CXCR5, CXCR6, CX3CR1 or CXCR1. For example, the second binding moiety of the bispecific antibody is Mogamulizumab or an antigen binding fragment thereof. Exemplary binding moieties which target cell-surface proteins such as cell surface protein expressed on immune cells or a tissue- or cell type-specific antigen include an antibody or antigen binding fragment targeting a checkpoint protein, such as an anti-PD1 antibody, an anti-PD-L1 antibody, or a CTLA-4 antibody. Suitable checkpoint protein targeting antibodies include Nivolumab, Pembrolizumab, Atezolizumab, Avelumab, Durvalumab, Cemiplimab, Dostarlimab, or Ipilimumab. In some embodiments, the second binding moiety of the bispecific antibody is a HER2 targeting antibody, such as Trastuzumab, Pertuzumab and/or Margetuximab. Exemplary cell surface molecules associated with T-cell activation include CD25, CTLA-4, PD-1, LAG3, TIGIT, ICOS, and TNF receptor super family members, 4-1BB, OX-40, and GITR.

### Effector Cells

In some embodiments, anti-CCR8 binding agents provided herein are linked, conjugated or fused to an effector cell. In some embodiments, an effector cell comprises an immune cell as described herein (e.g., NK cell, dendrite, B cell, macrophage, and the like) or a T cell expressing a chimeric antigen receptor (CAR T cell) engineered for chemokine receptor or CCR8 targeting. Recently, CAR T cells have gained attention from their clinical successes and expedited FDA approvals, cf. WO2020102240, incorporated herein in its entirety. In the CAR T cell approach, T cells are collected from patient blood and are then genetically engineered to express CARs that are specific for an antigen present on tumor cells. These engineered T cells are then re-administered to the same patient. Upon injection, CAR T cells recognize the targeted antigen on target cells to induce target cell death. CAR T cells thus constitute a novel modality for medical uses such as tumor treatment. The chimeric antigen receptor (CAR) is a genetically engineered receptor that is designed to target a specific antigen, for example, a tumor antigen. This targeting can result in cytotoxicity against the tumor, for example, such that CAR T cells expressing CARs can target and kill tumors via the specific tumor antigens. According to the present disclosure, the anti-CCR8 binding agent as described herein provided for CCR8 or chemokine receptor recognition can be used to engineer CAR T cells for specific recognition of CCR8 expressing cells or cells expressing the respective chemokine receptor. CARs encompassed herein can comprise a) a recognition region, e.g., a single chain fragment variable (scFv) region derived from a provided anti-CCR8 or anti-chemokine receptor antibody for recognition and binding to the CCR8 or chemokine receptor expressed by the target cell, and b) an activation signaling domain, e.g., the CD3 chain of T cells, which can serve as a T cell activation signal in CARs.

In some embodiments, CARs provided herein comprise a co-stimulation domain (e.g., CD137, CD28 or CD134) to achieve prolonged activation of T cells in vivo. Addition of a co-stimulation domain enhances the in vivo proliferation and survival of T cells containing CARs, and initial clinical data have shown that such constructs are promising therapeutic agents in the treatment of diseases, such as cancer. IN some embodiments, CAR T cells provided herein are useful in methods as described herein. For example, such CAR T cells can be used to treat any disease with local or systemic aberrant presence of cells expressing the target chemokine receptor, in particular CCR8 expressing cells, such as Treg cells.

### Heterologous Proteins

In some embodiments, anti-CCR8 binding agents provided herein are linked, conjugated or fused to a heterologous protein or polypeptide (or fragment thereof, for example, to a polypeptide (e.g., of about 10, about 20, about 30, about 40, about 50, about 60, about 70, about 80, about 90, or about 100 amino acids). In some embodiments, the linking, conjugation and/or fusion of heterologous proteins to an anti-CCR8 agent of the present disclosure generates fusion proteins, accordingly, provided herein are fusion proteins as well as uses thereof. In some embodiments, described herein are fusion proteins comprising an antigen-binding fragment of a CCR8 binding agent (e.g., an antibody), including a human CCR8 binding agent, described herein (e.g., comprising CDR1, CDR2, and/or CDR3 of VH and/or VL) and a heterologous protein, polypeptide, or peptide. In some embodiments, the heterologous protein, polypeptide, or peptide that a CCR8 binding agent (e.g., an antibody) is linked to is useful for targeting the CCR8 binding agent to a particular cell (e.g., a CCR8-expressing cell, including a tumor cell). In some embodiments, the heterologous protein is a signal peptide. In some embodiments, the heterologous protein is a cell penetrating peptide. In some embodiments, the heterologous protein is a subcellular localization signal.

### Cytolytic Agents

In some embodiments, anti-CCR8 binding agents provided herein are linked, conjugated or fused to one or more cytolytic agents. As used herein, a cytolytic agent is a moiety that reduces the proliferative capacity of one or more cells. A cell has reduced proliferative capacity when the cell becomes less able to proliferate, for example, because the cell undergoes apoptosis or otherwise dies, the cell fails to proceed through the cell cycle and/or fails to divide, the cell differentiates, etc. Nonlimiting exemplary cytolytic agents include, but are not limited to, radioisotopes, photosensitizers (PS), cytotoxins, and chemotherapeutic agents.

In some embodiments, an anti-CCR8 agent provided herein can be linked, fused or conjugated to one or more radioisotopes, also referred to herein as radionuclides. Exemplary radionuclides include: a beta particle, an alpha particle, or an Auger electron emitter. Suitable beta emitters are for example yttrium-90, iodine-131, strontium-89-chloride, lutetium-177, holmium-166, rhenium-186, rhenium-188, copper-67, promethium-149, gold-199, and rhodium-105. Suitable Auger electron emitters are for example bromine-77, indium-111, iodine-123, and iodine-125. Suitable alpha emitters are for example thorium-227, bismuth-213, radium-223, actinium-225 and astatine-211.

For example, thorium-227 (227Th) can be efficiently complexed with octadentate 3,2-hydroxypyridinone (3,2-HOPO) chelators that are conjugated to anti-CCR8 binding agents according to the current disclosure, resulting in highly stable targeted thorium-227 conjugates (TTCs). Targeted thorium conjugates (TTCs) comprise three main building blocks. Following the P-particle decay of actinium-227, the first building block, α-particle-emitting radionuclide 227Th is purified by ion exchange chromatography. The second building block is a chelator, such as a siderophore-derived chelator containing HOPO groups bearing four 3-hydroxy-N-methyl-2-pyridinone moieties on a symmetrical polyamine scaffold functionalized with a carboxylic acid linker for bioconjugation. Conjugation to a targeting moiety can be achieved through the amide bond formation with the F-amino groups of lysine residues. These octadentate 3,2-HOPO chelators can be efficiently labeled with 227Th, with high yield, purity, and stability at ambient conditions. Compared with the tetra-azacyclododecane-1,4,7,10-tetraacetic acid (DOTA) chelator, which often requires heating, the HOPO chelators are superior due to efficient radiolabeling at ambient temperatures and high stability of formed complexes. The third building block is the targeting moiety, that is the anti-CCR8 agent provided herein.

In some embodiments, an anti-CCR8 agent provided herein can be linked, fused or conjugated to one or more photosensitizers (PS). Photodynamic therapy (PDT) is a non-invasive treatment that involves the accumulation of a PS in solid tumors followed by the localized delivery of light of the correct wavelength to cause activation of the PS, which, in the presence of oxygen, leads to the in situ generation of reactive oxygen species (ROS) that cause damage to cellular components and, ultimately, necrosis or apoptosis. PDT is a promising tool in oncology but is frequently limited by side-effects caused by inadequate targeting of the photosensitizer. Accordingly, encompassed by the present disclosure is the conjugation of PS's to tumor-specific binding agents (e.g., antibodies). Also provided herein is the use of antigen-binding Ab fragments, e.g., Fab or scFv fragments, as, in some embodiments, antigen binding fragments retain the same binding specificity as full-size antibodies, but are more efficient at penetrating tumor masses due to their smaller size and are more effectively cleared from the circulation because of the lack of an Fc domain. In some embodiments, provided are porphyrins for use in the field of photodynamic therapy and photodiagnosis, and are one of the most prominent classes of photosensitizer in these areas of biomedical science (Sandland J, Boyle R W. Bioconjug Chem. 30(4):975-993 (2019)). In some embodiments, the photosensitizer is a tetrapyrrolic macrocycle. In some embodiments, the tetrapyrrolic macrocycle is a porphyrin, a chlorin, a bacteriochlorin, or a phthalocyanine.

In some embodiments, an anti-CCR8 agent provided herein can be linked, fused or conjugated to one or more cytotoxic agents, which can in some embodiments from antibody drug conjugate (ADC), referred to an anti-CCR8 ADC herein. One skilled in the art can select a suitable cytotoxin according to the intended application. In some embodiments, the cytolytic agent is at least one of an anti-metabolite, an alkylating agent, an antibiotic, a growth factor, a cytokine, an anti-angiogenic agent, an anti-mitotic agent, an anthracycline, toxin, or an apoptotic agent.

In some embodiments, the cytotoxic agent is an auristatin, a maytansinoid, a kinesin-spindle protein (KSP) inhibitor, a nicotinamide phosphoribosyltransferase (NAMPT) inhibitor or a pyrrolobenzodiazepine derivative. Generation of conjugates comprising maytansinoid may occur as described in Chari, Ravi V J, et al. Cancer research 52.1 (1992): 127-131, or EP2424569 B1, both incorporated herein in their entirety. Generation of conjugates comprising kinesin-spindle protein (KSP) inhibitors may occur as described in WO2019243159 A1, incorporated herein in its entirety. Generation of conjugates comprising a nicotinamide phosphoribosyltransferase (NAMPT) inhibitor may occur as described in WO2019149637 A1, incorporated herein in its entirety. Generation of conjugates comprising a pyrrolobenzodiazepine may be obtained as described in EP3355935 A1, incorporated herein in its entirety.

The cytotoxic and/or cytostatic agent of the anti-CCR8 ADC may be any agent known to inhibit the growth and/or replication of, and/or kill cells. Numerous agents having cytotoxic and/or cytostatic properties are known in the literature. Non-limiting examples of classes of cytotoxic and/or cytostatic agents include, by way of example and not limitation, cell cycle modulators, apoptosis regulators, kinase inhibitors, protein synthesis inhibitors, alkylating agents, DNA cross-linking agents, intercalating agents, mitochondria inhibitors, nuclear export inhibitors, topoisomerase I inhibitors, topoisomerase II inhibitors, RNA/DNA antimetabolites and antimitotic agents.

The linkers linking the cytotoxic and/or cytostatic agent(s) to the antigen binding moiety of an anti-CCR8 ADC may be long, short, flexible, rigid, hydrophilic or hydrophobic in nature, or may comprise segments that have different characteristics, such as segments of flexibility, segments of rigidity, etc. The linker may be chemically stable to extracellular environments, for example, chemically stable in the blood stream, or may include linkages that are not stable and release the cytotoxic and/or cytostatic agents in the extracellular milieu. In some embodiments, the linkers include linkages that are designed to release the cytotoxic and/or cytostatic agents upon internalization of the anti-CCR8 ADC, within the cell. In some specific embodiments, the linkers include linkages designed to cleave and/or immolate or otherwise breakdown specifically or non-specifically inside cells. A wide variety of linkers useful for linking drugs to antigen binding moieties such as antibodies in the context of ADCs are known in the art. Any of these linkers, as well as other linkers, may be used to link the cytotoxic and/or cytostatic agents to the antigen binding moiety of the anti-CCR8 ADCs, described herein.

The number of cytotoxic and/or cytostatic agents linked to the antigen binding moiety of an anti-CCR8 ADC (drug-to-antibody ratio: DAR) can vary and will be limited only by the number of available attachments sites on the antigen binding moiety and the number of agents linked to a single linker. Typically, a linker will link a single cytotoxic and/or cytostatic agent to the antigen binding moiety of anti-CCR8 ADC. In embodiments of anti-CCR8 ADC, which include more than a single cytotoxic and/or cytostatic agent, each agent may be the same or different. As long as the anti-chemokine receptor or anti-CCR8 ADC, does not exhibit unacceptable levels of aggregation under the conditions of use and/or storage, anti-CCR8 ADCs, with DARs of twenty, or even higher, are contemplated. In some embodiments, the anti-CCR8 ADC s, described herein may have a DAR in the range of about 1-10, 1-8, 1-6, or 1-4. In certain specific embodiments, the anti-CCR8 ADC may have a DAR of 2, 3 or 4. In some embodiments, the anti-CCR8 ADCs, are compounds according to structural formula (1):

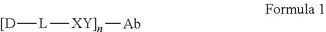

or salts thereof, where each “D” represents, independently of the others, a cytotoxic and/or cytostatic agent; each “L” represents, independently of the others, a linker; “Ab” represents an anti-CCR8 receptor binding moiety, e.g. an anti-CCR8 antibody provided herein; each “XY” represents a linkage formed between a functional group Rx on the linker and a “complementary” functional group Ry on the anti-chemokine receptor binding moiety; and n represents the DAR of the anti-chemokine receptor ADC.

In a specific exemplary embodiment, the anti-CCR8 ADCs are compounds according to structural formula (1) in which each “D” is the same and is either a cell-permeating auristatin (for example, dolastatin-10 or MMAE) or a cell-permeating minor groove-binding DNA cross-linking agent; each “L” is the same and is a linker cleavable by a lysosomal enzyme; each “XY” is a linkage formed between a maleimide and a sulfhydryl group; “Ab” is an antibody or fragment thereof comprising six CDRs corresponding to the six CDRs of an anti-chemokine receptor or CCR8 antibody according to the current disclosure; and n is 2, 3 or 4. In a specific embodiment “Ab” is a fully human antibody comprising human derived CDRs.

Cytotoxic and cytostatic agents are agents known to inhibit the growth and/or replication of and/or kill cells and in particular tumor cells or intra-tumoral Treg cells. These compounds may be used in a combination therapy with an anti-chemokine receptor antibody such as a CCR8 antibody, or as part of an anti-chemokine receptor ADC as described herein: In some embodiments, the drug moiety of the anti-chemokine receptor or anti-CCR8 ADC is a cytostatic agent selected from radionuclides, alkylating agents, DNA cross-linking agents, DNA intercalating agents (e.g., groove binding agents such as minor groove binders), cell cycle modulators, apoptosis regulators, kinase inhibitors, protein synthesis inhibitors, mitochondria inhibitors, nuclear export inhibitors, topoisomerase I inhibitors, topoisomerase II inhibitors, RNA/DNA antimetabolites and antimitotic agents. In some embodiments, the drug moiety of the anti-chemokine receptor or anti-CCR8 ADC is an alkylating agent selected from asaley (L-Leucine, N-[N-acetyl-4-[bis-(2-chloroethyl)amino]-DL-phenylalanyl]-, ethylester); AZQ (1,4-cyclohexadiene-1,4-dicarbamic acid, 2,5-bis(1-aziridinyl)-3,6-dioxo-, diethyl ester); BCNU (N,N′-Bis(2-chloroethyl)-N-nitrosourea); busulfan (1,4-butanediol dimethanesulfonate); (carboxyphthalato)platinum; CBDCA (cis-(1,1-cyclobutanedicarboxylato)diammineplatinum(II))); CCNU (N-(2-chloroethyl)-N′-cyclohexyl-N-nitrosourea); CHIP (iproplatin; NSC 256927); chlorambucil; chlorozotocin (2-[[[(2-chloroethyl) nitrosoamino]carbonyl]amino]-2-deoxy-D-glucopyranose); cis-platinum (cisplatin); clomesone; cyanomorpholinodoxorubicin; cyclodisone; dianhydrogalactitol (5,6-diepoxydulcitol); fluorodopan ((5-[(2-chloroethyl)-(2-fluoroethy)amino]-6-methyl-uracil); hepsulfam; hycanthone; indolinobenzodiazepine dimer DGN462; melphalan; methyl CCNU ((1-(2-chloroethyl)-3-(trans-4-methylcyclohexane)-1-nitrosourea); mitomycin C; mitozolamide; nitrogen mustard ((bis(2-chloroethyl) methylamine hydrochloride); PCNU ((1-(2-chloroethyl)-3-(2,6-dioxo-3-piperidyl)-1-nitrosourea)); piperazine alkylator ((1-(2-chloroethyl)-4-(3-chloropropyl)-piperazine dihydrochloride)); piperazinedione; pipobroman (N,N′-bis(3-bromopropionyl) piperazine); porfiromycin (N-methylmitomycin C); spirohydantoin mustard; teroxirone (triglycidylisocyanurate); tetraplatin; thio-tepa (N,N′,N″-tri-1,2-ethanediylthio phosphoramide); triethylenemelamine; uracil nitrogen mustard (desmethyldopan); Yoshi-864 ((bis(3-mesyloxy propyl)amine hydrochloride).

In some embodiments, the drug moiety of anti-CCR8 ADC is a DNA alkylating-like agent selected from Cisplatin; Carboplatin; Nedaplatin; Oxaliplatin; Satraplatin; Triplatin tetranitrate; Procarbazine; altretamine; dacarbazine; mitozolomide; temozolomide.

In some embodiments, the drug moiety of the anti-chemokine receptor or anti-CCR8 ADC is an alkylating antineoplastic agents selected from Carboquone; Carmustine; Chlornaphazine; Chlorozotocin; Duocarmycin; Evofosfamide; Fotemustine; Glufosfamide; Lomustine; Mannosulfan; Nimustine; Phenanthriplatin; Pipobroman; Ranimustine; Semustine; Streptozotocin; ThioTEPA; Treosulfan; Triaziquone; Triethylenemelamine; Triplatin tetranitrate.

In some embodiments, the drug moiety of anti-CCR8 ADC is a DNA replication and repair inhibitor selected from Altretamine; Bleomycin; Dacarbazine; Dactinomycin; Mitobronitol; Mitomycin; Pingyangmycin; Plicamycin; Procarbazine; Temozolomide; ABT-888 (veliparib); olaparib; KU-59436; AZD-2281; AG-014699; BSI-201; BGP-15; INO-1001; ONO-2231.

In some embodiments, the drug moiety of the anti-CCR8 ADC is a cell cycle modulator, such as Paclitaxel; Nab-Paclitaxel; Docetaxel; Vincristine; Vinblastine; ABT-348; AZD-1152; MLN-8054; VX-680; Aurora A-specific kinase inhibitors; Aurora B-specific kinase inhibitors and pan-Aurora kinase inhibitors; AZD-5438; BMI-1040; BMS-032; BMS-387; CVT-2584; flavopyridol; GPC-286199; MCS-5A; PD0332991; PHA-690509; seliciclib (CYC-202, R-roscovitine); ZK-304709; AZD4877, ARRY-520: GSK923295A.

In some embodiments, the drug moiety of the anti-CCR8 ADC is an apoptosis regulator such as AT-101 ((-)gossypol); G3139 or oblimersen (Bcl-2-targeting antisense oligonucleotide); IPI-194; IPI-565; N-(4-(4-((4′-chloro(1,1′-biphenyl)-2-yl)methyl)piperazin-1-ylbenzoyl)-4-(((1R)-3-(dimethylamino)-1-((phenylsulfanyl)methyl)propyl)amino)-3-nitrobenzenesulfonamide); N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide; GX-070 (Obatoclax®; 1H-Indole, 2-(2-((3,5-dimethyl-1H-pyrrol-2-yl)methylene)-3-methoxy-2H-pyrrol-5-yl)-)); HGS1029; GDC-0145; GDC-0152; LCL-161; LBW-242; venetoclax; agents that target TRAIL or death receptors (e.g., DR4 and DR5) such as ETR2-STO1, GDC0145, HGS-1029, LBY-135, PRO-1762; drugs that target caspases, caspase-regulators, BCL-2 family members, death domain proteins, TNF family members, Toll family members, and/or NF-kappa-B proteins.

In some embodiments, the drug moiety of the anti-CCR8 ADC is an angiogenesis inhibitor such as ABT-869; AEE-788; axitinib (AG-13736); AZD-2171; CP-547,632; IM-862; pegaptamib; sorafenib; BAY43-9006; pazopanib (GW-786034); vatalanib (PTK-787, ZK-222584); sunitinib; SU-11248; VEGF trap; vandetanib; ABT-165; ZD-6474; DLL4 inhibitors.

In some embodiments, the drug moiety of the anti-CCR8 ADC is a proteasome inhibitor such as Bortezomib; Carfilzomib; Epoxomicin; Ixazomib; Salinosporamide A.

In some embodiments, the drug moiety of the anti-chemokine receptor or anti-CCR8 ADC is a kinase inhibitor such as Afatinib; Axitinib; Bosutinib; Crizotinib; Dasatinib; Erlotinib; Fostamatinib; Gefitinib; Ibrutinib; Imatinib; Lapatinib; Lenvatinib; Mubritinib; Nilotinib; Pazopanib; Pegaptanib; Sorafenib; Sunitinib; SU6656; Vandetanib; Vemurafenib; CEP-701 (lesaurtinib); XL019; INCB018424 (ruxolitinib); ARRY-142886 (selemetinib); ARRY-438162 (binimetinib); PD-325901; PD-98059; AP-23573; CCI-779; everolimus; RAD-001; rapamycin; temsirolimus; ATP-competitive TORC1/TORC2 inhibitors including PI-103, PP242, PP30, Torin 1; LY294002; XL-147; CAL-120; ONC-21; AEZS-127; ETP-45658; PX-866; GDC-0941; BGT226; BEZ235; XL765.

In some embodiments, the drug moiety of the anti-CCR8 ADC is a protein synthesis inhibitor such as Streptomycin; Dihydrostreptomycin; Neomycin; Framycetin; Paromomycin; Ribostamycin; Kanamycin; Amikacin; Arbekacin; Bekanamycin; Dibekacin; Tobramycin; Spectinomycin; Hygromycin B; Paromomycin; Gentamicin; Netilmicin; Sisomicin; Isepamicin; Verdamicin; Astromicin; Tetracycline; Doxycycline; Chlortetracycline; Clomocycline; Demeclocycline; Lymecycline; Meclocycline; Metacycline; Minocycline; Oxytetracycline; Penimepicycline; Rolitetracycline; Tetracycline; Glycylcyclines; Tigecycline; Oxazolidinone; Eperezolid; Linezolid; Posizolid; Radezolid; Ranbezolid; Sutezolid; Tedizolid; Peptidyl transferase inhibitors; Chloramphenicol; Azidamfenicol; Thiamphenicol; Florfenicol; Pleuromutilins; Retapamulin; Tiamulin; Valnemulin; Azithromycin; Clarithromycin; Dirithromycin; Erythromycin; Flurithromycin; Josamycin; Midecamycin; Miocamycin; Oleandomycin; Rokitamycin; Roxithromycin; Spiramycin; Troleandomycin; Tylosin; Ketolides; Telithromycin; Cethromycin; Solithromycin; Clindamycin; Lincomycin; Pirlimycin; Streptogramins; Pristinamycin; Quinupristin/dalfopristin; Virginiamycin.

In some embodiments, the drug moiety of the anti-CCR8 ADC is a histone deacetylase inhibitor such as Vorinostat; Romidepsin; Chidamide; Panobinostat; Valproic acid; Belinostat; Mocetinostat; Abexinostat; Entinostat; SB939 (pracinostat); Resminostat; Givinostat; Quisinostat; thioureidobutyronitrile (Kevetrin™); CUDC-10; CHR-2845 (tefinostat); CHR-3996; 4SC-202; CG200745; ACY-1215 (rocilinostat); ME-344; sulforaphane.

In some embodiments, the drug moiety of the anti-CCR8 ADC is a topoisomerase I inhibitor such as camptothecin; various camptothecin derivatives and analogs (for example, NSC 100880, NSC 603071, NSC 107124, NSC 643833, NSC 629971, NSC 295500, NSC 249910, NSC 606985, NSC 74028, NSC 176323, NSC 295501, NSC 606172, NSC 606173, NSC 610458, NSC 618939, NSC 610457, NSC 610459, NSC 606499, NSC 610456, NSC 364830, and NSC 606497); morpholinisoxorubicin; SN-38.

In some embodiments, the drug moiety of the anti-CCR8 ADC is a topoisomerase II inhibitor such as doxorubicin; amonafide (benzisoquinolinedione); m-AMSA (4′-(9-acridinylamino)-3′-methoxymethanesulfonanilide); anthrapyrazole derivative ((NSC 355644); etoposide (VP-16); pyrazoloacridine ((pyrazolo[3,4,5-kl]acridine-2(6H)-propanamine, 9-methoxy-N, N-dimethyl-5-nitro-, monomethanesulfonate); bisantrene hydrochloride; daunorubicin; deoxydoxorubicin; mitoxantrone; menogaril; N,N-dibenzyl daunomycin; oxanthrazole; rubidazone; teniposide.

In some embodiments, the drug moiety of the anti-CCR8 ADC is a DNA intercalating agent such as anthramycin; chicamycin A; tomaymycin; DC-81; sibiromycin; pyrrolobenzodiazepine derivative; SGD-1882 ((S)-2-(4-aminophenyl)-7-methoxy-8-(3 S)-7-methoxy-2-(4-methoxyphenyl)-5-oxo-5,11a-dihydro-1H-benzo[e]pyrrolo[1,2-a][1,4]diazepin-8-yl)oxy)propoxy)-1H-benzo[e]pyrrolo[1,2-a][1,4]diazepin-5(11aH)-one); SG2000 (SJG-136; (11aS,11a'S)-8,8′-(propane-i,3-diylbis(oxy))bis(7-methoxy-2-methylene-2,3-dihydro-1H-benzo[e]pyrrolo[1,2-a][1,4]diazepin-5(11aH)-one)).

In some embodiments, the drug moiety of the anti-CCR8 ADC is a RNA/DNA antimetabolite such as L-alanosine; 5-azacytidine; 5-fluorouracil; acivicin; aminopterin derivative N-[2-chloro-5[[(2,4-diamino-5-methyl-6-quinazolinyl)methyl]amino]benzoyl]L-aspartic acid (NSC 132483); aminopterin derivative N-[4-[[(2,4-diamino-5-ethyl-6-quinazolinyl)methyl]amino]benzoyl]L-aspartic acid; aminopterin derivative N-[2-chloro-4-[[(2,4-diamino-6-pteridinyl)methyl]amino]benzoyl]L-aspartic acid monohydrate; antifolate PT523 ((Na-(4-amino-4-deoxypteroyl)-Ny-hemiphthaloyl-L-ornithine)); Baker's soluble antifol (NSC 139105); dichlorallyl lawsone ((2-(3,3-dichloroallyl)-3-hydroxy-1,4-naphthoquinone); brequinar; ftorafur ((pro-drug; 5-fluoro-1-(tetrahydro-2-furyl)-uracil); 5,6-dihydro-5-azacytidine; methotrexate; methotrexate derivative (N-[[4-[[(2,4-diamino-6-pteridinyl)methyl]methylamino]-1-naphthalenyl]carbonyl]L-glutamic acid); PALA ((N-(phosphonoacetyl)-L-aspartate); pyrazofurin; trimetrexate.

In some embodiments, the drug moiety of the anti-CCR8 ADC is a DNA antimetabolite such as 3-HP; 2′-deoxy-5-fluorouridine; 5-HP; α-TGDR (α-2′-deoxy-6-thioguanosine); aphidicolin glycinate; ara C (cytosine arabinoside); 5-aza-2′-deoxycytidine; β-TGDR (0-2′-deoxy-6-thioguanosine); cyclocytidine; guanazole; hydroxyurea; inosine glycodialdehyde; macbecin II; pyrazoloimidazole; thioguanine; thiopurine.

In some embodiments, the drug moiety of the anti-CCR8 ADC, is a mitochondria inhibitor such as pancratistatin; phenpanstatin; rhodamine-123; edelfosine; d-alpha-tocopherol succinate; compound 110; aspirin; ellipticine; berberine; cerulenin; GX015-070 (Obatoclax®; 1H-Indole, 2-(2-((3,5-dimethyl-1H-pyrrol-2-yl)methylene)-3-methoxy-2H-pyrrol-5-yl)-); celastrol (tripterine); metformin; Brilliant green; ME-344.

In some embodiments, the drug moiety of the anti-CCR8 ADC, is an antimitotic agent such as allocolchicine; auristatins, such as MMAE (monomethyl auristatin E) and MMAF (monomethyl auristatin F); halichondrin B; cemadotin; colchicine; cholchicine derivative (N-benzoyl-deacetyl benzamide); dolastatin-10; dolastatin-15; maytansine; maytansinoids, such as DM1 (N2′-deacetyl-N2′-(3-mercapto-1-oxopropyl)-maytansine); rhozoxin; paclitaxel; paclitaxel derivative ((2′-N-[3-(dimethylamino)propyl]glutaramate paclitaxel); docetaxel; thiocolchicine; trityl cysteine; vinblastine sulfate; vincristine sulfate.

In some embodiments, the drug moiety of the anti-CCR8 ADC is a nuclear export inhibitor such as callystatin A; delactonmycin; KPT-185 (propan-2-yl (Z)-3-[3-[3-methoxy-5-(trifluoromethyl)phenyl]-1,2,4-triazol-1-yl]prop-2-enoate); kazusamycin A; leptolstatin; leptofuranin A; leptomycin B; ratjadone; Verdinexor ((Z)-3-[3-[3,5-bis(trifluoromethyl)phenyl]-1,2,4-triazol-1-yl]-N-pyridin-2-ylprop-2-enehydrazide).

In some embodiments, the drug moiety of the anti-CCR8 ADC is a hormonal therapeutics such as anastrozole; exemestane; arzoxifene; bicalutamide; cetrorelix; degarelix; deslorelin; trilostane; dexamethasone; flutamide; raloxifene; fadrozole; toremifene; fulvestrant; letrozole; formestane; glucocorticoids; doxercalciferol; sevelamer carbonate; lasofoxifene; leuprolide acetate; megesterol; mifepristone; nilutamide; tamoxifen citrate; abarelix; prednisone; finasteride; rilostane; buserelin; luteinizing hormone releasing hormone (LHRH); Histrelin; trilostane or modrastane; fosrelin; goserelin.

Any of these agents that include, or that may be modified to include, a site of attachment to an antibody and/or binding fragment can be included in an anti-CCR8 ADC.

Other post-translational modifications include marker or “tag” sequences, such as a peptide, to facilitate purification. In some embodiments, the marker or tag amino acid sequence is a hexa-histidine peptide (SEQ ID NO: 74), such as the tag provided in a pQE vector (see, e.g., QIAGEN, Inc.), among others, many of which are commercially available. For example, as described in Gentz et al., Proc. Natl. Acad. Sci. USA 86:821-24, 1989, hexa-histidine (SEQ ID NO: 74) provides for convenient purification of a fusion protein. Other peptide tags useful for purification include, but are not limited to, the hemagglutinin (“HA”) tag, which corresponds to an epitope derived from the influenza hemagglutinin protein (Wilson et al., Cell 37:767-78, 1984), and the “FLAG” tag.

Methods for linking or conjugating (directly or indirectly) moieties (including polypeptides) to anti-CCR agents (e.g., anti-CCR8 antibodies) are well known in the art, any one of which can be used to make a modified anti-CCR8 agent, including ADCs and/or fusion proteins described herein.

II. Generating and expressing Anti-CCR8 Binding Agents

### A. Nucleic Acids and Vectors

Provided herein are nucleic acids and isolated nucleic acids comprising polynucleotides that encoding anti-CCR8 binding agents (e.g., anti-CCR8 antibodies, antigen-binding fragments thereof, and binding polypeptides that target the same). Nucleic acid molecules comprising polynucleotides that encode one or more chains of an anti-CCR8 binding agent are provided herein. In some embodiments, a nucleic acid molecule comprises a polynucleotide that encodes a heavy chain or a light chain of an anti-CCR8 binding agent. In some embodiments, a nucleic acid molecule comprises both a polynucleotide that encodes a heavy chain and a polynucleotide that encodes a light chain, of an anti-CCR8 binding agent. In some embodiments, a first nucleic acid molecule comprises a first polynucleotide that encodes a heavy chain and a second nucleic acid molecule comprises a second polynucleotide that encodes a light chain.

In some embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an anti-CCR8 binding agent comprises a nucleotide sequence that encodes at least one of the CDRs provided herein. In some embodiments, a polynucleotide encoding a heavy chain or light chain of an anti-CCR8 binding agent comprises a nucleotide sequence that encodes at least 3 of the CDRs provided herein. In some embodiments, a polynucleotide encoding a heavy chain or light chain of an anti-CCR8 binding agent comprises a nucleotide sequence that encodes at least 6 of the CDRs provided herein. In some embodiments, a polynucleotide encoding a heavy chain or light chain of an anti-CCR8 binding agent comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

In some embodiments, the nucleic acid is one that encodes for any of the amino acid sequences for the anti-CCR8 binding agent provided herein. In some embodiments, the nucleic acid is one that is at least 80% identical to a nucleic acid encoding any of the amino acid sequences for the antibodies in TABLES 2-4 described herein, for example, at least 80, 85, 90, 91, 92, 93, 94, 95, 96, 97, 98, or 99% identical. In some embodiments, nucleic acids provided herein comprise a nucleotide sequence that encodes an anti-CCR8 binding agent (e.g., anti-CCR8 antibodies, antigen-binding fragments thereof, and binding polypeptides that target the same), or a component (e.g., a domain or region) thereof as described herein. In some embodiments, nucleic acids provided herein comprise a nucleotide sequence that encodes any one or more of the amino acid sequences set forth in TABLES 2-4. In some embodiments, a nucleic acid provided herein comprises a nucleotide sequence that encodes any one or more of the CDR sequences set forth in TABLE 2. In some embodiments, a nucleic acid provided herein comprises a nucleotide sequence that encodes any one or more of the VH and/or VL sequences set forth in TABLE 3. In some embodiments, a nucleic acid provided herein comprises a nucleotide sequence that encodes any one or more of the HC and/or LC sequences set forth in TABLE 4.

In some embodiments, the nucleic acid is one that hybridizes to any one or more of the nucleic acid sequences provided herein. In some of the embodiments, the hybridization is under moderate conditions. In some embodiments, the hybridization is under highly stringent conditions, such as: at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C.

In some embodiments, one or more vectors (e.g., expression vectors) may comprise one or more nucleic acids for expression of the one or more polypeptides provided herein in a suitable host cell. In some embodiments, a vector comprises a first nucleic acid sequence encoding a heavy chain and a second nucleic acid sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv. In some embodiments, a first vector comprises a nucleic acid that encodes a heavy chain and a second vector comprises a nucleic acid that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

Vectors described herein are useful, for example, for amplifying the nucleic acids in host cells to create useful quantities thereof, and for expressing anti-CCR8 binding agents, such as antibodies or antibody fragments, using recombinant techniques.

In some embodiments, one or more vectors are expression vectors wherein one or more nucleic acids encoding anti-CCR8 binding agent sequences are operatively linked to one or more nucleic acids comprising expression control sequences. Autonomously replicating recombinant expression constructs such as plasmid and viral DNA vectors incorporating one or more nucleic acids encoding antibody sequences that bind CCR8 are specifically contemplated. Expression control DNA sequences include promoters, enhancers, and operators, and are generally selected based on the expression systems in which the expression construct (e.g., expression vector) is to be utilized. Promoter and enhancer sequences are generally selected for the ability to increase gene expression, while operator sequences are generally selected for the ability to regulate gene expression. Expression constructs (e.g., expression vectors) may also include sequences encoding one or more selectable markers that permit identification of host cells bearing the construct. Expression constructs (e.g., expression vectors) may also include sequences that facilitate, and preferably promote, homologous recombination in a host cell. In some embodiments, expression constructs (e.g., expression vectors) can also include sequences necessary for replication in a host cell. Commonly, expression vectors will contain selection markers, e.g., tetracycline, neomycin, and dihydrofolate reductase, to permit detection of those cells transformed with the desired DNA sequences.

Exemplary expression control sequences include promoter/enhancer sequences, including, for example, cytomegalovirus promoter/enhancer (Lehner et al., J. Clin. Microbiol., 29: 2494-2502, 1991; Boshart et al., Cell, 41: 521-530, 1985); Rous sarcoma virus promoter (Davis et al., Hum. Gene Ther., 4: 151, 1993); Tie promoter (Korhonen et al., Blood, 86(5): 1828-1835, 1995); simian virus 40 promoter; DRA (downregulated in adenoma; Alrefai et al., Am. J. Physiol. Gastrointest. Liver Physiol., 293: G923-G934, 2007); MCT1 (monocarboxylate transporter 1; Cuff et al., Am. J. Physiol. Gastrointet. Liver Physiol., G977-G979. 2005); and Math1 (mouse atonal homolog 1; Shroyer et al., Gastroenterology, 132: 2477-2478, 2007), for expression in mammalian cells, the promoter being operatively linked upstream (e.g., 5′) of a polypeptide coding sequence. In another variation, the promoter is an epithelial-specific promoter or endothelial-specific promoter. Nucleic acids may also optionally include a suitable polyadenylation sequence (e.g., the SV40 or human growth hormone gene polyadenylation sequence) operably linked downstream (e.g., 3′) of the polypeptide coding sequence.

If desired, the one or more nucleic acids also optionally comprise nucleotide sequences encoding secretory signal peptides fused in frame with the polypeptide sequences. The secretory signal peptides direct secretion of the antibody polypeptides by the cells that express the one or more nucleic acids, and are cleaved by the cell from the secreted polypeptides. The one or more nucleic acids may further optionally comprise sequences whose only intended function is to facilitate large scale production of the vector. One can manufacture and administer nucleic acids for gene therapy using procedures that have been described in the literature for a variety of transgenes. See, e.g., Isner et al., Circulation, 91: 2687-2692 (1995); and Isner et al., Human Gene Therapy, 7: 989-1011 (1996).

In some embodiments, nucleic acids may further comprise additional sequences to facilitate uptake by host cells and expression of the antibody or fragment thereof (and/or any other peptide). In some embodiments, a “naked” transgene encoding an antibody or fragment thereof described herein (e.g., a transgene without a viral, liposomal, or other vector to facilitate transfection) is employed.

Any suitable vectors may be used to introduce one or more nucleic acids that encode an anti-CCR8 binding agent into the host. Exemplary vectors that have been described include replication deficient retroviral vectors, including but not limited to lentivirus vectors (see, e.g., Kim et al., J. Virol., 72(1): 811-816 (1998); Kingsman & Johnson, Scrip Magazine, October, 1998, pp. 43-46); parvoviral vectors, such as adeno-associated viral (AAV) vectors (U.S. Pat. Nos. 5,474,9351; 5,139,941; 5,622,856; 5,658,776; 5,773,289; 5,789,390; 5,834,441; 5,863,541; 5,851,521; 5,252,479; Gnatenko et al., J. Invest. Med., 45: 87-98, (1997)); adenoviral (AV) vectors (see, e.g., U.S. Pat. Nos. 5,792,453; 5,824,544; 5,707,618; 5,693,509; 5,670,488; 5,585,362; Quantin et al., Proc. Natl. Acad. Sci. USA, 89: 2581-2584 (1992); Stratford Perricaudet et al., J. Clin. Invest., 90: 626-630 (1992); and Rosenfeld et al., Cell, 68: 143-155, (1992)); an adenoviral adeno-associated viral chimeric (U.S. Pat. No. 5,856,152) or a vaccinia viral or a herpesviral vector (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237); and combinations thereof. Optionally, viral vectors are rendered replication-deficient by, for example, deleting or disrupting select genes required for viral replication.

Any of these expression vectors can be prepared using standard recombinant DNA techniques described in, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

Other non-viral delivery mechanisms contemplated include calcium phosphate precipitation (Graham and Van Der Eb, Virology, 52: 456-467 (1973); Chen and Okayama, Mol. Cell Biol., 7: 2745-2752, 1987; Rippe et al., Mol. Cell Biol., 10: 689-695 (1990)) DEAE-dextran (Gopal, Mol. Cell Biol., 5: 1188-1190, (1985)), electroporation (Tur-Kaspa et al., Mol. Cell Biol., 6: 716-718 (1986); Potter et al., Proc. Nat. Acad. Sci. USA, 81: 7161-7165 (1984)), direct microinjection (Harland and Weintraub, J. Cell Biol., 101: 1094-1099 (1985), DNA-loaded liposomes (Nicolau and Sene, Biochim. Biophys. Acta, 721: 185-190 (1982); Fraley et al., Proc. Natl. Acad. Sci. USA, 76: 3348-3352 (1979); Felgner, Sci Am., 276(6): 102-6, 1997; Felgner, Hum Gene Ther., 7(15): 1791-3 (1996)), cell sonication (Fechheimer et al., Proc. Natl. Acad. Sci. USA, 84: 8463-8467 (1987)), gene bombardment using high velocity microprojectiles (Yang et al., Proc. Natl. Acad. Sci USA, 87: 9568-9572 (1990)), and receptor-mediated transfection (Wu and Wu, J. Biol. Chem., 262: 4429-4432 (1987); Wu and Wu, Biochemistry, 27: 887-892 (1988); Wu and Wu, Adv. Drug Delivery Rev., 12: 159-167 (1993)).

An expression vector (or an antibody or fragment thereof described herein) may be entrapped in a liposome. See, e.g., Ghosh and Bachhawat, In: Liver diseases, targeted diagnosis and therapy using specific receptors and ligands, Wu G, Wu C ed., New York: Marcel Dekker, pp. 87-104 (1991); Radler et al., Science, 275(5301): 810-814 (1997). Also contemplated are various commercial approaches involving “lipofection” technology. In some embodiments, the liposome may be complexed with a hemagglutinating virus (HVJ). This has been shown to facilitate fusion with the cell membrane and promote cell entry of liposome-encapsulated DNA (see, e.g., Kaneda et al., Science, 243: 375-378 (1989). In some embodiments, the liposome is complexed or employed in conjunction with nuclear nonhistone chromosomal proteins (HMG-1) (see, e.g., Kato et al., J. Biol. Chem., 266: 3361-3364 (1991). In some embodiments, the liposomes are complexed or employed in conjunction with both HVJ and HMG-1. Such expression constructs have been successfully employed in transfer and expression of nucleic acid in vitro and in vivo. In some embodiments, a CCR8 binding agent (e.g., an antibody), including a human CCR8 binding agent, is included in the liposome to target the liposome to cells (such as tumor cells) expressing CCR8 on their surface.

### B. Host Cells, Anti-CCR8 Production, and Isolation

Provided herein is a cell, such as a host cell, or a use thereof. A cell described herein, and specifically a host cell described herein can refer to a cell that is used to receive, maintain, reproduce and amplify a vector as provided herein. A host cell also can be used to comprise an anti-CCR8 binding agent provided herein, or express an anti-CCR8 binding agent provided herein, e.g. an antibody or fragment thereof, encoded by the vector. The nucleic acid contained in the vector is replicated when the host cell divides, thereby amplifying the nucleic acids.

For recombinant production of an anti-CCR8 binding agent, nucleic acids encoding the anti-CCR8 binding agent, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the anti-CCR8 binding agent) or produced by recombinant methods or obtained by chemical synthesis.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

Suitable host cells for cloning or expression of anti-CCR8 binding agent-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, N.J., pp. 245-254 (2003), describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for anti-CCR8 binding agent-encoding vectors, including fungi and yeast strains whose glycosylation pathways have been “humanized”, resulting in the production of an anti-CCR8 binding agent (e.g., an antibody) with a partially or fully human glycosylation pattern. See Gemgross, T. U., Nat. Biotech. 22:1409-1414 (2004); and Li, H. et al., Nat. Biotech. 24:210-215 (2006).

Suitable host cells for the expression of (glycosylated) anti-CCR8 binding agent are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36:59-74 (1977)) or Epi293 cells as used herein; baby hamster kidney cells (BHK); mouse sertoli cells (TM4 cells as described, e.g., in Mather, J. P., Biol. Reprod. 23: 243-252 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383: 44-68 (1982)); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77: 4216-4220 (1980)); and myeloma cell lines such as YO, NSO and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, N.J., pp. 255-268 (2004). In some embodiments, the host cell is eukaryotic, e.g., a Chinese Hamster Ovary (CHO) cell or lymphoid cell (e.g., YO, NSO, Sp20 cell).

In some embodiments, cells provided herein comprise anti-CCR8 binding agents as described herein. In some embodiments, provided herein is a chimeric antigen receptor (CAR) comprising any one of the isolated anti-CCR8 binding agents disclosed herein, an antigen-binding portion thereof, or a variant or derivative thereof. A CAR can refer to an artificial T cell surface receptor that is engineered to be expressed on an immune effector cell and specifically bind an antigen. CARs may be used as a therapy with adoptive cell transfer. For example, monocytes are removed from a patient (blood, tumor or ascites fluid) and modified so that they express the receptors specific to a particular form of antigen. Accordingly, in some embodiments, the CARs have been expressed with specificity to a tumor associated antigen. In some embodiments, CARs provided herein specifically to an epitope of CCR8 outside the N-terminal domain of CCR8. In some embodiments, the CAR further comprises a transmembrane domain. In some embodiments, the CAR further comprises an intracellular signaling domain. In some embodiments, the CAR further comprises a hinge region and/or a spacer region. In some embodiments, provided herein is T cell receptor (TCR) comprising an antigen-binding region disclosed herein that binds specifically to an epitope of CCR8 outside the N-terminal domain of CCR8. In some embodiments, the TCR further comprises a transmembrane domain. In some embodiments, the TCR further comprises an intracellular signaling domain. In some embodiments, CARs and/or TCRs provided herein comprise fused anti-CCR8 agents comprising single-chain variable fragments (scFv) derived monoclonal antibodies, fused to CD3-zeta transmembrane and intracellular domain. The specificity of CAR designs may be derived from ligands of receptors (e.g., peptides). In some embodiments, a CAR can target cancers by redirecting a monocyte/macrophage expressing the CAR specific for tumor associated antigens.

In some embodiments, an anti-CCR8 agent that specifically binds to in an intracellular epitope (ICD) (e.g., a C-terminal epitope) of CCR8 and not to an extracellular epitope (e.g., N-terminal epitope) of CCR8 can be generated by immunizing a subject (e.g., a non-human subject) with an immunogen comprising a cell that expresses the targeted epitope (e.g., a C-terminal epitope), an extracellular epitope (e.g., a N-terminal epitope) and an antibody or antigen-binding fragment thereof that specifically binds to the extracellular epitope (e.g., a N-terminal epitope) for a sufficient amount of time to permit the subject to generate the anti-CCR8 agent that specifically binds to the targeted epitope (e.g., the C-terminal epitope). Accordingly, in some embodiments, also provided herein are methods and/or systems of producing an anti-CCR8 agent that specifically binds to a C-terminal epitope of CCR8 and not to a N-terminal epitope of CCR8.

Anti-CCR8 binding agents provided herein can be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region or Fc region and to purify an antibody or fusion protein. Hydrophobic interactive chromatography, for example, a butyl or phenyl column, may also suitable for purifying some polypeptides. Ion exchange chromatography (for example anion exchange chromatography and/or cation exchange chromatography) may also suitable for purifying some polypeptides. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides. Many methods of purifying polypeptides are known in the art.

In some embodiments, an anti-CCR8 binding agent is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### III. Systems

Provided herein are systems that can be useful for detecting CCR8, diagnosing a CCR8 related disease or disorder, mediating activity in a diseased microenvironment characterized by the expression of CCR8 and/or CCR8 expressing cells, treatments of a CCR8 related disease or disorder, or any combination thereof. Systems provided herein comprise components, wherein the components comprise: any one or more of the anti-CCR8 agents provided herein, including anti-CCR8 antibodies, antigen-binding fragments thereof, variants thereof, derivatized versions thereof, and binding polypeptides directed to the same, nucleic acids encoding the same, and/or vectors comprising such nucleic acids.

In some embodiments, systems provided herein further comprise a sample obtained from a subject. A sample is biological sample. In some embodiments, a sample is obtained from a subject which is in need thereof of treatment with an anti-CCR8 provided herein. In some embodiments, a sample is a tumor sample. In some embodiments, a sample is a solid tumor sample.

In some embodiments, systems provided herein further comprise one or more system solutions comprising compositions, pharmaceutical compositions, buffers, reagents including detection and/or amplification reagents, or any combination thereof. Such compositions, pharmaceutical compositions, buffers, and reagents are further described herein. In some embodiments, systems provided herein further comprise one or more support mediums, containers and other articles of manufacture, or any combination thereof. Such support mediums, containers and other articles of manufacture are further described herein.

In some embodiments, system components described herein are each comprised in a composition, or any combination of components are comprised in single composition. In some embodiments, system components described herein are each comprised in a container, or any combination of components are comprised in single container. In some embodiments, system components described herein are each comprised in a device, or any combination of components are comprised in single device. In some embodiments, a container is comprised in a device, and can be referred to as a device component, for example as a reaction chamber. In some embodiments, a system solution is comprised in a device and can be referred to as a device component, for example, as a detection reagent.

Unless specified otherwise, systems comprise kits. In some embodiments, the systems comprising kits are referred to as kits. Unless specified otherwise, systems comprise devices. In some embodiments, the systems comprising the devices are referred to as devices. In some embodiments, systems described herein are provided in the form of a companion diagnostic assay or device, a point-of-care assay or device, or an over-the-counter diagnostic assay/device. Unless specified otherwise, systems described herein are used in methods for detecting the presence of CCR8 and/or CCR8 expressing cells.

### Kits and Articles of Manufacture

Also provided herein are kits comprising anti-CCR8 binding agents (e.g., an anti-CCR8 antibody, antigen-binding fragment thereof, and binding polypeptide targeting the same) or uses thereof.

An anti-CCR8 binding agent (e.g., an anti-CCR8 antibody, antigen-binding fragment thereof, and binding polypeptide targeting the same) described herein may be provided in a kit, for example, a packaged combination of reagents in predetermined amounts with instructions for use (e.g., instructions for performing a diagnostic assay; instructions for performing a laboratory assay). In some embodiments, the kit is a diagnostic kit configured to detect CCR8 and/or CCR8 expressing cells in a sample (e.g., a biological sample). Where the anti-CCR8 antibody, antigen binding fragment thereof, or agent is labeled with a fluorophore, the kit may include an identical isotype negative control irrelevant antibody to control for non-specific binding of the anti-CCR8 antibody, antigen-binding fragment thereof, or agent. Where the anti-CCR8 antibody, antigen-binding fragment thereof, or agent is labeled with an enzyme, the kit may include substrates and cofactors required by the enzyme (e.g., substrate precursor which provides the detectable chromophore or fluorophore). Additional additives may be included such as stabilizers, buffers (e.g., a block buffer or lysis buffer), and the like. The relative amounts of the various reagents may be varied widely to provide for concentrations in solution of the reagents that substantially optimize the sensitivity of the assay. In embodiments, reagents may be provided as dry powders (e.g., lyophilized powder), including excipients that on dissolution will provide a reagent solution having the appropriate concentration.

Further provided herein is an article of manufacture containing materials useful for the treatment, or diagnosis, of the disorders described herein is provided. An article of manufacture may comprise a container and a label. Suitable containers include, for example, bottles, vials, syringes, and test tubes. Containers may be formed from a variety of materials such as glass or plastic. A container may hold a composition that is effective for treating a condition and may have a sterile access port (for example the container may be an intravenous solution bag or a vial having a stopper pierceable by a hypodermic injection needle). Such compositions can comprise anti-CCR8 binding agents (e.g., an anti-CCR8 antibody, antigen-binding fragment thereof, and binding polypeptide targeting the same) provided herein, for example, as an active agent. A label on, or associated with, the container indicates that the composition is used for treating, or diagnosing a condition of choice. The article of manufacture may further comprise a second container comprising a pharmaceutically acceptable buffer, such as phosphate-buffered saline, Ringer's solution and dextrose solution; and may further include other materials desirable from a commercial and user standpoint, including other buffers, diluents, filters, needles, syringes, and package inserts with instructions for use. Articles of manufacture may be provided independently, or provided together as a kit, for use in methods and systems described herein.

### Detection, Diagnostics, and Monitoring

Provided herein are the use of anti-CCR8 antibodies in a system, method, composition, kit, or device of detecting or diagnosing.

In some embodiments, provided herein are systems, methods, compositions, kits or devices to detect CCR8 and/or CCR8 expressing cells in a sample obtained from a subject. In some embodiments, detecting CCR8 and/or CCR8 expressing cells in a sample obtained from a subject comprises contacting the sample with an anti-CCR8 binding agent described herein (e.g., anti-CCR8 antibodies, antigen-binding fragment thereof, and binding polypeptides targeting the same) under conditions and for a period of time sufficient to permit binding between the anti-CCR8 binding agent and CCR8 and/or CCR8 expressing cells in the sample, and detecting the absence, presence, and/or abundance of CCR8 and/or CCR8 expressing cells in said sample. In some embodiments, detecting the absence, presence, and/or abundance of CCR8 in said sample comprises detecting if binding has occurred between anti-CCR8 binding agent and CCR8 and/or CCR8 expressing cells, thereby forming anti-CCR8 binding agent:CCR8 and/or CCR8 expressing cell complexes. Also provided herein are compositions and reagents comprising anti-CCR8 binding agents described herein (e.g., anti-CCR8 antibodies, antigen-binding fragment thereof, and binding polypeptides targeting the same) for use in systems, methods, kits and/or devices of detecting CCR8 and/or CCR8 expressing cells in a sample obtained from a subject.

In some embodiments, also provided herein are methods, systems, and compositions for the monitoring and/or quantifying of CCR8 expression and/or CCR8 expressing cells in a subject. In some embodiments, monitoring and/or quantifying CCR8 expression and/or CCR8 expressing cells in a sample obtained from a subject comprises contacting the sample with an anti-CCR8 binding agent described herein (e.g., anti-CCR8 antibodies, antigen-binding fragment thereof, and binding polypeptides targeting the same) under conditions and for a period of time sufficient to permit binding between the anti-CCR8 binding agent and CCR8 in the sample, and detecting the presence, absence, and/or abundance of CCR8 expression and/or CCR8 expressing cells in said sample, and determining the difference between CCR8 expression and/or CCR8 expressing cells in the sample obtained from the subject and a predetermined baseline control. In some embodiments, the predetermined baseline control is obtained from a subject prior to treatment with an anti-CCR8 antibody, such as a therapeutic anti-CCR8 antibody provided herein. In some embodiments, monitoring and/or quantifying CCR8 expression and/or CCR8 expressing cells are useful in methods of treatments as described herein.

In some embodiments, the sample is not known to contain CCR8 expression and/or CCR8 expressing cells. In some embodiments, the sample obtained from the subject is suspected or known to contain CCR8 expression and/or CCR8 expressing cells. A sample obtained from the subject can be a biological sample. A biological sample can be a sample of a cells, fluid or tissue obtained from a subject, including, blood, saliva, urine, and other bodily solutions, fat, muscle, lymph, organ tissue (including tonsils), glands (including thymus tissue), and cancer or tumor tissue, or tissue suspected to be cancerous or tumorous. In some embodiments, detection of CCR8 and/or CCR8 expressing cells includes the detection of CCR8 and/or CCR8 expressing cells in certain components of the sample but not in other components where not present or abundant. For example, in some embodiments, anti-CCR8 binding agents (e.g., anti-CCR8 antibodies, antigen-binding fragment thereof, and binding polypeptides targeting the same) can be used to detect CCR8 in a heterogeneous population of cells, wherein CCR8 can be detected in certain cells of the population (e.g., immune cells such as lymphocytes) but not significantly detected in other cells (e.g., non-immune cells). Methods of detecting can be in vitro methods or in vivo methods. Detection of CCR8 in immune cells if further described herein.

In some embodiments, anti-CCR8 binding agents provided herein (e.g., anti-CCR8 antibodies, antigen-binding fragment thereof, and binding polypeptides targeting the same) can be used to select subjects eligible for therapy with anti-CCR8 binding agents described herein (e.g., anti-CCR8 antibodies, antigen-binding fragment thereof, and binding polypeptides targeting the same), where CCR8 is a biomarker for selection of subjects. In such embodiments, upon detecting CCR8 and/or CCR8 expressing cells in a sample obtained from said subject, such as CCR8 expressed on an immune cell, it can be indicated that the subject is eligible for therapy with anti-CCR8 binding agents described herein.

In some embodiments, methods provided herein comprise determining the expression level of CCR8 in various patient or subject populations to provide expression profiles. In some embodiments, methods provided herein determine the expression level of CCR8 and/or the presence of CCR8 expressing cells in different tissues of patient or subject populations to provide expression profiles. In some embodiments, CCR8 expression levels and/or CCR8 expressing cells in certain patient or subject populations and/or tissues can be used as a baseline to measure CCR8 presence, absence, or abundance in a sample obtained from a test subject. In such embodiments, an increased level of CCR8 test expression and/or level of CCR8 expressing cells relative to a baseline expression can be indicate that the subject is eligible for therapy with anti-CCR8 binding agents described herein.

In some embodiments, anti-CCR8 binding agents provided herein (e.g., anti-CCR8 antibodies, antigen-binding fragment thereof, and binding polypeptides targeting the same) can be used to monitor the progress of subjects being treated with anti-CCR8 agent (e.g., anti-CCR8 antibody) therapy, where depletion of CCR8 is a biomarker for the progress of subjects being treated with anti-CCR8 agent (e.g., anti-CCR8 antibody) therapy. In such embodiments, upon detecting the depletion of CCR8 in a sample obtained from said subject, such as depletion of CCR8 expressed on an immune cell and/or depletion of CCR8 expressing cells, it can be indicated that the prognosis of a subject being treated with anti-CCR8 agent (e.g., anti-CCR8 antibody) therapy has improved.

In some embodiments, anti-CCR8 binding agents provided herein (e.g., anti-CCR8 antibodies, antigen-binding fragment thereof, and binding polypeptides targeting the same) can be used to monitor the effectiveness of anti-CCR8 agent (e.g., anti-CCR8 antibody) treatment in a subject, where depletion of CCR8 and/or CCR8 expressing cells is a biomarker for the effectiveness of the anti-CCR8 agent (e.g., anti-CCR8 antibody) treatment in the subject. In such embodiments, upon detecting the depletion of CCR8 and/or CCR8 expressing cells in a sample obtained from said subject, such as depletion of CCR8 expressed on an immune cell and/or depletion of CCR8 expressing cells, it can be indicated that the anti-CCR8 agent (e.g., anti-CCR8 antibody) treatment is effective. Treatment can be initial treatment with the anti-CCR8 agent (e.g., anti-CCR8 antibody) or any treatment thereof.

In some embodiments, methods provided herein comprise determining the expression level of CCR8 in a subject or subject populations before treatment with a therapeutic anti-CCR8 agent (e.g., anti-CCR8 antibody) to provide baseline CCR8 expression profiles of a subject or subject populations before treatment. In some embodiments, methods provided herein determine the expression level of CCR8 and/or the presence of CCR8 expressing cells in a tissue of a subject or tissues of subject populations before treatment with a therapeutic anti-CCR8 agent (e.g., anti-CCR8 antibody) to provide baseline CCR8 expression and/or CCR8 expressing cell profiles of a tissue or of tissues of subject populations before treatment. In some embodiments, CCR8 expression levels in subject, subject population or tissues thereof can be used as a baseline to measure CCR8 and/or CCR8 expressing cell presence, absence, or abundance in a sample obtained from a test subject, which can be the same subject as the baseline or a member of the subject population. In such embodiments, a depleted level of CCR8 expression and/or depleted level of CCR8 expressing cells relative to a baseline expression can be indicate that the subject is responding to treatment with therapeutic anti-CCR8 agent (e.g., anti-CCR8 antibody), such as therapeutic anti-CCR8 binding agents described herein. Accordingly, also provided herein are methods, systems, and compositions for the monitoring the prognosis of a subject receiving treatment with a therapeutic anti-CCR8 agent (e.g., anti-CCR8 antibody). Treatment can be initial treatment with the anti-CCR8 agent (e.g., anti-CCR8 antibody) or any treatment thereof.

In some embodiments, methods, systems, and compositions provided herein are useful to measure the depletion of CCR8 expression or CCR8 expressing cells in methods, systems, and/or compositions to monitor the effectiveness of a therapeutic anti-CCR8 binding agent treatment and/or the prognosis of a subject receiving the therapeutic anti-CCR8 binding agent treatment. In some embodiments, methods, systems, and compositions comprised herein can measure the depletion of CCR8 expression or CCR8 expressing cells of less than 5%, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or by more than 95% relative to the amount of CCR8 expression or the amount of CCR8 expressing cells of a baseline control. In some embodiments, the CCR8 expressing cell is a Treg, such as a Treg in a tumor microenvironment.

In some embodiments, anti-CCR8 binding agents described herein (e.g., anti-CCR8 antibodies, antigen-binding fragment thereof, and binding polypeptides targeting the same) comprises a detectable label or moiety. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels and moieties are described in further detail herein. In some embodiments, an anti-CCR8 binding agent described herein (e.g., an anti-CCR8 antibody, antigen-binding fragment thereof, and binding polypeptide targeting the same) is conjugated or fused to a detectable label or moiety. Suitable labels include radioisotopes, fluorescent labels, enzyme-substrate labels, and the like. In some embodiments, the detectable label or moiety is a detectable label or moiety that is suitable for immunohistochemistry.

Radioisotopes include 35S, 14C, 125I, 3H, and 131I. The anti-CCR8 binding agent can be labeled with the radioisotope using the techniques described in Current Protocols in Immunology, Volumes 1 and 2, Coligen et al, Ed. Wiley-Interscience, New York, N.Y., Pubs. (1991), for example, and radioactivity can be measured using scintillation counting.

Fluorescent labels include rare earth chelates (europium chelates) or fluorescein and its derivatives, rhodamine and its derivatives, dansyl, Lissamine, phycoerythrin, Texas Red and Brilliant Violet™ are available. The fluorescent labels can be conjugated to the anti-CCR8 binding agent using the techniques disclosed in Current Protocols in Immunology, supra, for example. Fluorescence can be quantified using a flow cytometer, imaging microscope or fluorimeter.

Enzyme-substrate labels are such that the enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemilluminometer, for example) or donates energy to a fluorescent acceptor.

Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclicoxidases (such as uricase and xanthine oxidase), lactoperoxidase, microperoxidase, and the like. Techniques for conjugating enzymes to antibodies are described in O'Sullivan et al., Methods in Enzym. (ed J. Langone & H. Van Vunakis), Academic press, New York, 73:147-166 (1981). U.S. Pat. No. 4,275,149 provides a review of some of the above-described labels.

Examples of enzyme-substrate combinations include, for example: (i) Horseradish peroxidase (HRP) with hydrogen peroxidase as a substrate, where the hydrogen peroxidase oxidizes a dye precursor (e.g., orthophenyl ene diamine (OPD) or 3,3′,5,5′-tetramethyl benzidine hydrochloride (TMB)); (ii) alkaline phosphatase (AP) with para-Nitrophenyl phosphate as chromogenic substrate; and (iii) b-D-galactosidase (b-D-Gal) with a chromogenic substrate (e.g., p-nitrophenyl\hat{ }-D-galactosidase) or fluorogenic substrate 4-methylumbelliferyl\hat{ }-D-galactosidase. Numerous other enzyme-substrate combinations may be used (e.g., U.S. Pat. Nos. 4,275,149 and 4,318,980, each of which is incorporated by reference herein).

In some embodiments, the label is indirectly conjugated with the anti-CCR8 binding agent described herein (e.g., an anti-CCR8 antibody, antigen-binding fragment thereof, and binding polypeptide targeting the same). An ordinary artisan in the art will be aware of various techniques for achieving this. For example, an antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In some embodiments, the anti-CCR8 binding agent described herein (e.g., an anti-CCR8 antibody, antigen-binding fragment thereof, and binding polypeptide targeting the same) need not be labeled, and the presence thereof can be detected, e.g., using a labeled antibody which binds to an anti-CCR8 antibody.

In some embodiments, an anti-CCR8 binding agent described herein (e.g., an anti-CCR8 antibody, antigen-binding fragment thereof, and binding polypeptide targeting the same) is immobilized on a solid support or substrate. In some embodiments, an anti-CCR8 binding agent herein is non-diffusively immobilized on a solid support (e.g., the anti-CCR8 antibody, antigen-binding fragment thereof, or the binding polypeptide targeting the same does not detach from the solid support). A solid support or substrate can be any physically separable solid to which an anti-CCR8 antibody, antigen-binding fragment thereof, or the binding polypeptide targeting the same can be directly or indirectly attached including, but not limited to, surfaces provided by microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™, polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, the solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, a solid support or substrate can be a collection of particles. In some embodiments, the particles can comprise silica, and the silica may comprise silica dioxide. In some embodiments the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe2+ and Fe3+). An anti-CCR8 binding agent described herein (e.g., an anti-CCR8 antibody, antigen-binding fragment thereof, and binding polypeptide targeting the same) may be linked to a solid support by covalent bonds or by non-covalent interactions and may be linked to a solid support directly or indirectly (e.g., via an intermediary agent such as a spacer molecule or biotin).

An anti-CCR8 binding agent described herein (e.g., an anti-CCR8 antibody, antigen-binding fragment thereof, and binding polypeptide targeting the same) may be employed in any known assay method, such as flow cytometry, immunohistochemistry, immunofluorescence, mass cytometry (e.g., Cytof instrument), competitive binding assays, direct and indirect sandwich assays, and immunoprecipitation assays. Zola, Monoclonal Antibodies: A Manual of Techniques, pp. 147-158 (CRC Press, Inc. 1987).

Flow cytometry and mass cytometry assays generally involve the use of a single primary antibody to specifically identify the presence of the target molecule expressed on the surface of a dispersed suspension of individual cells. The dispersed cells are typically obtained from a biological fluid sample, e.g., blood, but may also be obtained from a dispersion of single cells prepared from a solid tissue sample such as spleen or tumor biopsy. The primary antibody may be directly conjugated with a detectable moiety, e.g., a fluorophore such as phycoerythrin for flow cytometry or a heavy metal chelate for mass cytometry. Alternatively, the primary antibody may be unlabeled or labeled with an undetectable tag such as biotin, and the primary antibody is then detected by a detectably labeled secondary antibody that specifically recognizes the primary antibody itself or the tag on the primary antibody. The labeled cells are then analyzed in an instrument capable of single cell detection, e.g., flow cytometer, mass cytometer, fluorescence microscope or brightfield light microscope, to identify those individual cells in the dispersed population or tissue sample that express the target recognized by the primary antibody. Detailed description of the technological basis and practical application of flow cytometry principles may be found in, e.g., Shapiro, Practical Flow Cytometry, 4th Edition, Wiley, 2003.

Sandwich assays involve the use of two antibodies, each capable of binding to a different immunogenic portion, or epitope, of the protein that is detected. In a sandwich assay, the test sample analyte is bound by a first antibody that is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme. In a cell ELISA, the target cell population may be attached to the solid support using antibodies first attached to the support and that recognize different cell surface proteins. These first antibodies capture the cells to the support. CCR8 on the surface of the cells can then be detected by adding anti-CCR8 antibody to the captured cells and detecting the amount of CCR8 antibody attached to the cells. In certain instances, fixed and permeabilized cells may be used, an in such instances, surface CCR8 and intracellular CCR8 may be detected.

For immunohistochemistry, the blood or tissue sample may be fresh or frozen or may be embedded in paraffin and fixed with a preservative such as formalin, for example.

The anti-CCR8 binding agents described herein may also be used for in vivo diagnostic assays. Generally, the antibody is labeled with a radionuclide (such as ^{lu}In, ″Tc, ¹⁴C, ^{13 X}I, ¹²⁵I, ³H, ³²P, or ³⁵S) so that the bound target molecule can be localized using immunoscintillography.

Also provided herein are anti-CCR8 binding agents (e.g., an anti-CCR8 antibody, antigen-binding fragment thereof, and binding polypeptide targeting the same), compositions and methods for detecting CCR8 in immune cells. Detection of CCR8 in immune cells may refer to detection on the surface of immune cells (e.g., by surface staining) and/or inside immune cells (e.g., by intracellular staining). In some embodiments, antibodies, antigen-binding fragments thereof, or agents and methods are provided for detecting CCR8 in a heterogeneous population of immune cells. A heterogeneous population of immune cells may comprise two or more types of immune cells. For example, a heterogeneous population of immune cells may comprise two or more B cells, plasmacytoid dendritic cells (pDCs), lymphocytes, leukocytes, T cells, monocytes, macrophages, neutrophils, myeloid dendritic cells (mDCs), innate lymphoid cells, mast cells, eosinophils, basophils, natural killer cells, and the like. In some embodiments, a heterogeneous population of immune cells comprises peripheral blood mononuclear cells (PBMCs) which may include, for example, T cells, B cells, natural killer cells, and monocytes.

Generally, cells are contacted with an anti-CCR8 antibody, antigen-binding fragment thereof, or agent described herein (e.g., in a flow cytometry assay as described in the Examples; or any suitable protein or cell detection assay). In some embodiments, CCR8 is detected at a significant level in certain immune cells by anti-CCR8 binding agents (e.g., an anti-CCR8 antibody, antigen-binding fragment thereof, and binding polypeptide targeting the same) described herein. CCR8 may be detected at a significant level by an anti-CCR8 binding agent described herein in certain immune cells and not significantly detected in other immune cells. The level of CCR8 detection in certain immune cells may vary according to certain factors such as, for example, type of detection assay, type of detection reagent (e.g., type of dye), antibody concentration, donor cell variability, and the like.

In some of any embodiments, any of the anti-CCR8 binding agents (e.g., an anti-CCR8 antibody, antigen-binding fragment thereof, and binding polypeptide targeting the same) provided herein can be used in the characterization of single cells by measurement of gene-expression levels and cellular proteins. Among such known single cell sequencing platforms suitable for integration with anti-CCR8 binding agents described herein, for example, the antibodies or antigen binding fragments thereof described herein, is the Drop-seq method, including, but not limited to, microfluidic, plate-based, or microwell, Seq-Well™ method and adaptations of the basic protocol, and InDrop™ method.

In another embodiment, a single cell sequencing platform suitable for integration with the antibodies or antigen binding fragments thereof described herein is 10× genomics single cell 3′ solution or single cell V(D)J solution, either run on Chromium controller, or dedicated Chromium single cell controller. Other suitable sequencing methods include Wafergen iCell8™ method, Microwell-seq method, Fluidigm Cl™ method and equivalent single cell products. Still other known sequencing protocols useful with the anti-CCR8 binding agents described herein include BD Resolve™ single cell analysis platform and ddSeq (from Illumina® Bio-Rad® SureCell™ WTA 3′ Library Prep Kit for the ddSEQ™ System, 2017, Pub. No. 1070-2016-014-B, Illumina Inc., Bio-Rad Laboratories, Inc.). In still other embodiment, the anti-CCR8 binding agents described herein are useful with combinatorial indexing based approaches (sci-RNA-seq™ method or SPLiT-seq™ method) and Spatial Transcriptomics, or comparable spatially resolved sequencing approaches. The methods and compositions described herein can also be used as an added layer of information on standard index sorting (FACS) and mRNA-sequencing-based approaches.

In some of any embodiments, any of the anti-CCR8 binding agents (e.g., an anti-CCR8 antibody, antigen-binding fragment thereof, and binding polypeptide targeting the same) described herein can be used to detect the presence, absence or amount of the various nucleic acids, proteins, targets, oligonucleotides, amplification products and barcodes described herein.

In some of any embodiments, the detection comprises hybridization of a detectable moiety to the anti-CCR8 binding agent, such as an anti-CCR8 antibody. In some of any embodiments, the sample is contacted with a second antibody. In some of any embodiments, the second antibody is an antibody comprising a detectable moiety. In some of any embodiments, the detectable moiety comprises an oligonucleotide. In some of any embodiments, the detectable moiety comprises a fluorescent label. In some of any embodiments, the measurement comprises sequencing. In some of any embodiments, the detectable moiety comprises immunofluorescence. In some of any embodiments, the sample is a formalin-fixed paraffin-embedded sample. In some of any embodiments, the sample comprises a cell. In some of any embodiments, the sample comprises a tissue sample.

Detection of CCR8 at a significant level may refer to a particular signal to noise (S:N) ratio (e.g., threshold or range) measured in a flow cytometry assay. In some embodiments, CCR8 is detected at a significant level in immune cells (e.g., in Treg cells).

In some embodiments, anti-CCR8 binding agents useful for detection, monitoring and/or diagnostic systems, methods and/or compositions provided herein comprise at least one, two, three, four, five, or six CDRs selected from (a) HCDR1 comprising the amino acid sequence of SEQ ID NO: 5, (b) HCDR2 comprising the amino acid sequence of SEQ ID NO: 6, (c) HCDR3 comprising the amino acid sequence of SEQ ID NO: 7, (d) LCDR1 comprising the amino acid sequence of SEQ ID NO: 8, (e) LCDR2 comprising the amino acid sequence of SEQ ID NO: 9, and (f) LCDR3 comprising the amino acid sequence of SEQ ID NO: 10.

### IV. Compositions

Provided herein are compositions that can be useful for detecting CCR8, diagnosing a CCR8 related disease or disorder, mediating activity in a diseased microenvironment characterized by the expression of CCR8 and/or CCR8 expressing cells, treatments of a CCR8 related disease or disorder, or any combination thereof. Compositions provided herein comprise: any one or more of the anti-CCR8 agents provided herein, including anti-CCR8 antibodies, antigen-binding fragments thereof, variants thereof, derivatized versions thereof, and binding polypeptides directed to the same, nucleic acids encoding the same, and/or vectors comprising such nucleic acids. Optionally, compositions comprise buffers, reagents, carriers, excipients, and stabilizers and described herein.

In some embodiments, composition provided herein comprise an anti-CCR8 agent at a concentration of more than about any one of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 125 mg/mL, 150 mg/mL, 175 mg/mL, 200 mg/mL, 225 mg/mL, or 250 mg/mL.

In some embodiments, a composition is a cell culture media comprising an anti-CCR8 agent. In some embodiments, a host cell culture fluid comprising an anti-CCR8 agent. In some embodiments, a composition is a detection reagent comprising an anti-CCR8 agent. In some embodiments, a composition is a pharmaceutical composition comprising an anti-CCR8 agent.

### Pharmaceutical Compositions

Provided herein are pharmaceutical compositions comprising any one or more of the anti-CCR8 agents provided herein, including anti-CCR8 antibodies, antigen-binding fragments thereof, variants thereof, derivatized versions thereof, and binding polypeptides directed to the same, nucleic acids encoding the same, and/or vectors comprising such nucleic acids, and a physiologically or pharmaceutically acceptable carrier, excipient or stabilizer e.g. according to Remington's Pharmaceutical Sciences (18th ed.; Mack Pub. Co.: Eaton, Pa., 1990), e.g. in the form of lyophilized formulations or aqueous solutions. In some embodiments, the anti-CCR8 agent or portion or derivative thereof has the desired degree of purity.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween®, Pluronic® or polyethylene glycol (PEG).

In some embodiments, compositions further comprise one or more co-treatments as further described herein.

### V. Methods of Use or Treatment

Provided herein are methods comprising the use of anti-CCR8 binding agents provided herein. Methods include the use of anti-CCR8 binding agents provided herein for the detection of CCR8. Methods of detection are further described herein.

Also provided herein is the use of anti-CCR8 binding agents described herein to mediate activity in a diseased microenvironment. In some embodiments, a diseased microenvironment is characterized by or associated with cells expressing chemokine receptors, such as CCR8+ cells, including CCR8+ immune cells (e.g., CCR8+ Treg cells). In some embodiments, mediating activity in a diseased microenvironment comprises inducing an immune response in said environment, altering the activity of one or more cell populations in said environment, or altering the amount of one or more cell populations in said environment. In some embodiments, inducing an immune response in the diseased microenvironment comprises inducing ADCC, CDC, and/or ADCP. In some embodiments, altering the activity of one or more cell populations in said environment comprises inducing one or more cell populations in said environment to activate, enhance and/or increase effector activity, such as activating, enhancing, and/or increasing granule polarization in NK cells. In some embodiments, altering the amount of one or more cell populations in said environment comprises depleting the amount of cells which promote disease growth and/or escape, such as regulatory T cells which promote tumor growth and/or tumor escape. In some embodiments, further provided herein is the use of anti-CCR8 binding agents described herein to mediate activity in a diseased microenvironment for the treatment of a tumor or cancer.

### A. Methods of Treatment

Further provided herein is an anti-CCR8 binding agent inducing ADCC, CDC and/or ADCP for use in the treatment of a tumor or cancer. Thus, provided herein is a method of treating a disease, the method comprising administering an effective amount or dose of the anti-CCR8 binding agent (e.g., anti-CCR8 antibodies, antigen-binding fragments thereof, and binding polypeptides that target the same), including modified agents thereof (e.g., conjugated anti-CCR8 antibodies), compositions or pharmaceutical compositions comprising the same, to a subject in need thereof. In some embodiments, the anti-CCR8 binding agent being administered in a therapeutic method described herein is referred to as a therapeutic anti-CCR8 binding agent.

Methods of treating can also comprise detecting CCR8 in a sample obtained from the subject being treated, and/or methods of diagnosing a disease or diseased microenvironment is characterized by or associated with cells expressing chemokine receptors, such as CCR8+ cells, including CCR8+ immune cells (e.g., CCR8+ Treg cells). Methods of detecting and/or diagnosing can be executed by using such methods provided herein, however, a person of ordinary skill in the art would understand that any suitable method could be utilized to detect CCR8 in a sample or diagnose disease characterized by or associated with cells expressing CCR8.

Accordingly, provided herein is a method of treatment comprising detecting the level of CCR8 expression and/or CCR8 expressing cells in a sample obtained from a subject with the use of an anti-CCR8 binding agents as described herein, wherein the subject has been administered a therapeutic anti-CCR8 antibody as described herein. In some embodiments, anti-CCR8 binding agents provided herein can be used to detect CCR8 expression or CCR8 expressing cells in a sample obtained from a subject in the presence of a therapeutic anti-CCR8 antibody described herein. In some embodiments, anti-CCR8 binding agents can be used to detect CCR8 expression or CCR8 expressing cells in such samples using an IHC assay. In some embodiments, anti-CCR8 binding agents can be used to detect CCR8 selectively over any one or combination of CCR1-7 and 9. In some embodiments, anti-CCR8 binding agents can be used to detect CCR8 selectively 1.5, 2, 2.5, 3, 3.5, 4, 4.5, 5, 5.5, 6, 6.5, 7, 7.5, 8, 8.5, 9, 9.5, or about 10 times more than a background signal or noise.

In some embodiments, therapeutic anti-CCR8 binding agents that can be used in methods provided herein comprise any suitable anti-CCR8 binding agent, including the anti-CCR8 binding agents provided herein. Alternatively, therapeutic anti-CCR8 binding agents provided herein can be any suitable commercially available anti-CCR8 binding agent. Exemplary therapeutic anti-CCR8 binding agents (e.g., anti-CCR8 antibodies) are described in the references set forth in the following paragraphs, the entire contents of each of which are incorporated by reference herein.

In some embodiments, therapeutic anti-CCR8 antibodies that can be used in the methods provided herein can include TPP-23411 (Bayer), TPP-29338, TPP-27454, TPP-31741 (Surface Oncology, Inc.), BMS-986340 (also known as 4A19 and TPP-31743; Bristol Myers Squibb), LM-108 (LaNova Medicines), S-531011 (Shionogi), FPA157 (Five Prime, Amgen), IPG-7236 (Immunophage Biomedical), ICP-BO5 (InnoCare Pharma Tech), SRF-114 (Surface Oncology), HBM1022 (Harbour BioMed), HFB1011 (HiFiBio), BAY-3375968 (Bayer), 10-1 (Oncurious), ZL-1218 (Zai Lab), GB2101 (Genor), PSB-114 (Sound Biologics), IPG-A05 (Immunophage Biomedical Co Ltd), PM-1024 (Biotheus Inc, Adimab LLC), DT-7012 (Domain Therapeutics SA), BCG-005 (Biocytogen LLC, Liberothera Co Ltd), GS-1811 (Jounce Therapeutics Inc, Gilead Sciences Inc), GNUV-202 (Genuv Inc), CHS-3318 (Coherus BioSciences Inc), CTM-033 (Lepu Biopharma), the anti-CCR8 monoclonal antibody by Integral Molecular Inc, the anti-CCR8 antibody by iBio Inc., the anti-CCR8 antibody by Bristol-Myers Squibb, the anti-CCR8 monoclonal antibody by iTeos therapeutics, the anti-CCR8 therapy by Flanders Institute for Biotechnology VZW and Oncurious NV, EGL-002 (Egle Therapeutics), BGB-A3055 (Beigene, Ltd.), ABBV-514 (Abbvie, Inc.), ABT-863 (Abilita Therapeutics, Inc.), and CHS-114 (Coherus Biosciences, Inc.).

In some embodiments, the therapeutic anti-CCR8 antibody includes azirkitug (AbbVie Inc), cafelkibart (LaNova Medicines), lanerkitug (Bayer), imzokitug (BMS), denikitug (GS-1811; Gilead Sciences Inc), or enzelkitug.

In some embodiments, the therapeutic anti-CCR8 antibody has a similar property as anti-CCR8 antibody TPP-23411. Antibodies with similar properties to TPP-23411 are disclosed in WO2024/052517 (pages 2-3), incorporated herein by reference.

In some embodiments, therapeutic anti-CCR8 antibodies that can be used in the methods provided herein are as described in WO2022078277, WO2022081718, WO2022000443, WO2022042690, WO2022003156, WO07044756, CN110835371, CN110835374, WO20138489, WO21142002, WO21152186, WO21163064, WO21178749, WO21194942, WO22136649, WO22136650, WO22136647, WO22241034, WO22256563, WO22256559, WO22268192, WO23288241, WO23010054, WO23020621, WO22211046, WO23098888, WO23116880, WO23137466, TW202330599, WO23174396, WO23193732, WO23208182, WO23206350, WO23219147, U.S. Ser. No. 11/427,640B1, US20210277129A1, US20230119066A1, US20230049152A1, US20220403037A1, U.S. Ser. No. 10/087,259B1, WO2013131010A2, WO2021183685A2, US20230176060A1, US20230101029A1, EP4118105A2, US20220389394A1, US20220365091A1, US20210324028A1, EP3589657A1, WO2018112032A1, EP4114862A2, WO2022216965A1, US20230270857A1, EP4232463A2, EP4225373A1, WO2023147488A1, WO2022200303A1, US20190092875A1, EP2656069A2, U.S. Pat. No. 6,416,954B1, WO2023125729A1, US20230160009A1, WO2023076574A1, US20230107291A1, WO2023046156A1, WO2023044402A1, WO2023036246A1, WO2022165260A1, WO2022256628A1, EP3458473B8, EP4090686A2, EP4081548A1, WO2022216702A1, WO2022192457A1, WO2022192895A1, US20220289838A1, EP4041758A1, EP4025255A1, US20220202950A1, EP3994270A1, EP3990476A1, WO2022063194A1, EP3917966A1, WO2021231327A2, EP3908601A1, U.S. Ser. No. 11/173,325B2, EP3793613A1, EP3762421A2, WO2020214718A1, EP2717941B1, U.S. Ser. No. 10/401,357B2, US20200215111A1, EP2652508B1, U.S. Pat. No. 9,233,120B2, U.S. Pat. No. 8,512,701B2, US20200157518A1, EP3554550A1, US20190255107A1, US20190153115A1, EP3471752A1, WO2019036043A2, US20190041389A1, US20180318417A1, US20170218091A1, WO2017011342A1, WO2015183837A1, US20150259418A1, EP2872170A2, WO2015037000A1, EP2654792A2, EP2655415A2, US20130004416A1, EP2337795A2, US20090220486A1, US20090118175A1, EP1948694A2, WO2007005605A2, WO2005010153A2, WO2002067771A2, WO2023010054A1, WO2022078277A1, WO2021194942A1, US20230049152, WO2021163063A1, WO2020128489A1, WO2021194942A1, WO2022000443A1, US20220001043A1, WO2024052517A1, U.S. Ser. No. 11/427,640B1, WO2021152186A2, WO2021260208A2, WO2021260206A2, WO2021260209A2, or WO2021260210A2. In some embodiments, the anti-CCR8 antibody includes an anti-CCR8 antibody described in Worden, F., et al. “Abstract CT038: Phase 1 study of anti-CCR8 antibody CHS-114 with and without anti-PD-1 antibody toripalimab in patients with advanced solid tumors.” Cancer Research 85(8), Supplement 1 (2025): CT038-CT038; Paustian, A. S., et al. ABBV-514: an afucosylated CCR8 specific antibody that targets and eliminates key immunosuppressive tumor regulatory T cells. Cancer Research 85(8), Supplement 1 (2025): 6025-6025; Zucchetti, A., et al. “Enhanced anti-tumor efficacy through prolonged plasma membrane retention of a novel anti-CCR8/IL2 mutein fusion antibody.” Cancer Research 85(8), Supplement 1 (2025): 3767-3767; Wang, X., et al. “BGB-A3055, an afucosylated anti-CCR8 antibody, preferentially depletes intratumoral regulatory T cells and inhibits tumor growth in preclinical models.” Cancer Research 85(8), Supplement 1 (2025): 4787-4787; Kuss, P., et al. “ABT-863: The next generation anti-CCR8 immuno-oncology antibody therapeutic.” Cancer Research 85(8), Supplement 1 (2025): 636-636; or Lelievre, H., et al. “Comprehensive characterization of DT-7012, a differentiated CCR8-depleting antibody for the treatment of solid tumors.” Cancer Research 85(8), Supplement 1 (2025): 312-312. Exemplary HC and LC sequences of therapeutic antibodies that can be used with the methods provided herein are listed in TABLE 5.

In some embodiments, therapeutic anti-CCR8 antibodies that can be used in the methods provided herein can be obtained from the hybridoma having ATCC Accession No. PTA-6940, PTA-6938, or PTA-6939.

In some embodiments, the therapeutic anti-CCR8 antibody that can be used in the methods provided herein is the HBM1022 antibody as disclosed in Lu et al. HBM1022, a novel anti-CCR8 antibody depletes tumor-infiltrating regulatory T cells via enhanced ADCC activity, mediates potent anti-tumor activity with Keytruda. Journal for ImmunoTherapy of Cancer 2020; 8:doi: 10.1136/jitc-2020-SITC2020.0711.

In some embodiments, the therapeutic anti-CCR8 antibody that can be used in the methods provided herein is the FPA157 antibody as disclosed in Rankin A, Naik E861 Development of FPA157, an anti-CCR8 depleting antibody engineered to preferentially eliminate tumor-infiltrating T regulatory cells. Journal for ImmunoTherapy of Cancer 2020; 8:doi: 10.1136/jitc-2020-SITC2020.0861.

In some embodiments, the therapeutic anti-CCR8 antibody that can be used in the methods provided herein is the SRFl 14 antibody as disclosed in Lake A, Warren M, Das S, et al. Journal for ImmunoTherapy of Cancer 2020; 8:doi: 10.1136/jitc-2020-SITC2020.0726.

In some embodiments, the therapeutic anti-CCR8 antibody that can be used in the methods provided herein is antibody is the anti-CCR8 hlgGl-nonfucosylated BMS-986340 antibody as disclosed in Lan, Ruth, et al. “Highly selective anti-CCR8 antibody-mediated depletion of regulatory T cells leads to potent antitumor activity alone and in combination with anti-PD-1 in preclinical models.” (2020): 6694-6694 and in Bayati F, Mohammadi M, Valadi M, Jamshidi S, Foma A M, Sharif-Paghaleh E. The Therapeutic Potential of Regulatory T Cells: Challenges and Opportunities. Front Immunol. 2021; 11:585819. Published 2021 Jan 15. doi: 10.3389/fimmu.2020.585819.

In some embodiments, the therapeutic anti-CCR8 antibody that can be used in the methods provided herein is the nanobody as disclosed in Van Damme H, Dombrecht B, Kiss M, Roose H, Allen E, Van Overmeire E, Kancheva D, Martens L, Murgaski A, Bardet P M R, Blancke G, Jans M, Bolli E, Martins M S, Elkrim Y, Dooley J, Boon L, Schwarze J K, Tacke F, Movahedi K, Vandamme N, Neyns B, Ocak S, Scheyltjens I, Vereecke L, Nana F A, Merchiers P, Laoui D, Van Ginderachter J A. Therapeutic depletion of CCR8+ tumor-infiltrating regulatory T cells elicits antitumor immunity and synergizes with anti-PD-1 therapy. J Immunother Cancer. 2021 February; 9(2):e001749. doi: 10.1136/j itc-2020-001749. PMID: 33589525; PMCID: PMC7887378.

In some embodiments, the therapeutic anti-CCR8 antibody is an antibody that includes any of the sequences disclosed in TABLE 6. In some embodiments, the therapeutic anti-CCR8 is an antibody that includes (a) a heavy chain complementary determining region 1 (HCDR1) having the amino acid sequence of SEQ ID NO: 53, (b) an HCDR2 having the amino acid sequence of SEQ ID NO: 54, (c) an HCDR3 having the amino acid sequence of SEQ ID NO: 55, (d) a light chain complementarity determining region 1 (LCDR1) having the amino acid sequence of SEQ ID NO: 56, (e) an LCDR2 having the amino acid sequence of SEQ ID NO: 57, and (f) an LCDR3 having the amino acid sequence of SEQ ID NO: 58. In some embodiments, the therapeutic anti-CCR8 antibody includes (a) a heavy chain variable region (VH) having at least 90%, 91%, 92%, 93%, 94%, 95%, 96%, 97%, 98%, 99%, or 100% sequence identity to the amino acid sequence of SEQ ID NO: 59; and (b) a light chain variable region (VL) having at least 90%, 91%, 92%, 93%, 94%, 95%, 96%, 97%, 98%, 99%, or 100% sequence identity to the amino acid sequence of SEQ ID NO: 60. In some embodiments, the therapeutic anti-CCR8 antibody includes (a) a heavy chain variable region (VH) having the amino acid sequence of SEQ ID NO: 59; and (b) a light chain variable region (VL) having the amino acid sequence of SEQ ID NO: 60. In some embodiments, the therapeutic anti-CCR8 antibody includes (a) a heavy chain having at least 90%, 91%, 92%, 93%, 94%, 95%, 96%, 97%, 98%, 99%, or 100% sequence identity to the amino acid sequence of SEQ ID NO: 61; and (b) a light chain having at least 90%, 91%, 92%, 93%, 94%, 95%, 96%, 97%, 98%, 99%, or 100% sequence identity to the amino acid sequence of SEQ ID NO: 62. In some embodiments, the therapeutic anti-CCR8 antibody includes (a) a heavy chain having the amino acid sequence of SEQ ID NO: 61; and (b) a light chain having the amino acid sequence of SEQ ID NO: 62. In some embodiments, the therapeutic anti-CCR8 antibody is afucosylated.

In some embodiments, the therapeutic anti-CCR8 antibody is denikitug.

Multiple modes of action can be envisioned for the anti-CCR8 binding agents provided herein. For example, mode of action is the conjugation of an anti-CCR8 binding agents thereof to a drug in the form of an antibody drug conjugate (ADC). Another mode of action is the ability of an anti-CCR8 binding agents to induce ADCC. A third mode of action resides in the ability of an anti-CCR8 binding agents to induce ADCP. A third mode of action resides in the ability of an anti-CCR8 binding agents to induce CDC. Modes of action are further described herein.

In some embodiments, an effective amount or dose of anti-CCR8 binding agents, refers to the amount of anti-CCR8 binding agent described herein, or the amount of a composition or pharmaceutical composition comprising said anti-CCR8 binding agent that will elicit the biological or medical response of or desired therapeutic effect on a tissue, system, animal, mammal, or human that is being sought by the researcher, medical doctor, or other clinician. An effective amount of the anti-CCR8 binding agent may vary according to factors such as the disease state, age, sex, and weight of the individual, and the ability of the TCE molecule to elicit a desired response in the individual. An effective amount is also one in which any toxic or detrimental effect of the anti-CCR8 binding agent is outweighed by the therapeutically beneficial effects. Such benefit includes improving signs or symptoms of cancer. An effective amount can be readily determined by one skilled in the art, by the use of known techniques, and by observing results obtained under analogous circumstances. An effective amount of a anti-CCR8 binding agent described herein may be administered in a single dose or in multiple doses. In determining the effective amount for a patient, a number of factors are considered by the attending medical practitioner, including, but not limited to: the patient's size (e.g., weight or mass), body surface area, age, and general health; the specific disease or disorder involved; the degree of, or involvement, or the seventy′ of the disease or disorder; the response of the individual patient; the particular compound administered; the mode of administration; the bioavailability characteristics of the preparation administered; the dose regimen selected; the use of concomitant medication; and other relevant circumstances known to medical practitioners.

For therapeutic applications, the anti-CCR8 binding agent can be administered to a patient or subject, e.g. to a human or non-human subject, in a pharmaceutically acceptable dosage form. For example, administration may occur intravenously as a bolus or by continuous infusion over a period of time, by intramuscular, intraperitoneal, intra-cerebrospinal, subcutaneous, intra-articular, intrasynovial, intrathecal, oral, topical, or inhalation routes. The anti-CCR8 binding agents and pharmaceutical compositions thereof according to the current disclosure are particularly suitable to be administered by intra-tumoral, peri-tumoral, intra-lesional, or peri-lesional routes, to exert local as well as systemic therapeutic effects.

Exemplary administration routes include parenteral (e.g., intramuscular, intravenous, intra-arterial, intraperitoneal, or subcutaneous), intrapulmonary and intranasal. In addition, the antibodies, fragments, conjugates and pharmaceutical compositions might be administered by pulse infusion, with, e.g., declining doses of the antibody, fragment or conjugate. Preferably, the dosing is given by injections, most preferably intravenous or subcutaneous injections, depending in part on whether the administration is brief or chronic. The amount to be administered may depend on a variety of factors such as the clinical symptoms, weight of the patient or subject, and whether other drugs are administered. The skilled artisan will recognize that the route of administration will vary depending on the disorder or condition to be treated.

An anti-CCR8 binding agent can be administered as needed to subjects. Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of an anti-CCR8 binding agent is administered to a subject one or more times. In some embodiments, an effective dose of an anti-CCR8 binding agent is administered to the subject once a month, less than once a month, such as, for example, every two months or every three months. In some embodiments, an effective dose of an anti-CCR8 binding agent is administered less than once a month, such as, for example, once every three weeks, once every two weeks, or once every week. An effective dose of an anti-CCR8 binding agent is administered to the subject at least once. In some embodiments, the effective dose of an anti-CCR8 binding agent may be administered multiple times, including for periods of at least a month, at least six months, or at least a year.

In some embodiments, pharmaceutical compositions are administered in an amount effective for treatment of (including prophylaxis of) cancer. The therapeutically effective amount is typically dependent on the weight of the subject being treated, his or her physical or health condition, the extensiveness of the condition to be treated, or the age of the subject being treated. In general, anti-CCR8 binding agents may be administered in an amount in the range of about 10 g/kg body weight to about 100 mg/kg body weight per dose. In some embodiments, anti-CCR8 binding agents may be administered in an amount in the range of about 50 g/kg body weight to about 5 mg/kg body weight per dose. In some embodiments, anti-CCR8 binding agents may be administered in an amount in the range of about 100 g/kg body weight to about 10 mg/kg body weight per dose. In some embodiments, anti-CCR8 binding agents may be administered in an amount in the range of about 100 g/kg body weight to about 20 mg/kg body weight per dose. In some embodiments, anti-CCR8 binding agents may be administered in an amount in the range of about 0.5 mg/kg body weight to about 20 mg/kg body weight per dose.

### B. Indications

In some embodiments, systems, compositions, and methods provided herein are useful for the treatment of a disease or disorder related to CCR8 expression or CCR8 expressing cells.

For example, the cancer may comprise tumor cells expressing CCR8, such as B cell lymphoma and T cell lymphoma. In the alternative, the cancer may comprise intra-tumoral Tregs expressing CCR8. CCR8 is upregulated in several tumor indications, such as T-cell acute lymphoblastic leukemia, breast cancer, triple-negative breast cancer, triple positive breast cancer, non-small cell lung cancer (NSCLC), small cell lung cancer (SCLC), testicular cancer, gastric cancer, head and neck squamous cell carcinoma, thymoma, esophageal adenocarcinoma, colorectal cancer, pancreatic adenocarcinoma, ovarian cancer or cervical cancer, acute myeloid leukemia, kidney cancer, bladder cancer, skin cancer, melanoma, thyroid cancer, mesothelioma, sarcoma and prostate cancer. According to some preferred embodiments, the use as a medicament is the use in the treatment of head and neck cancer, breast cancer, gastric cancer, lung cancer, squamous cell carcinoma, esophageal tumor, melanoma, bladder cancer, liver cancer, and/or prostate cancer.

In some embodiments, the tumor is selected from Adrenal cancer (e.g. Adrenocortical carcinoma or Pheochromocytoma), Bladder cancer (e.g. Transitional cell carcinoma, Transitional cell carcinoma-Papillary), Brain cancer (e.g. Glioma-Astrocytoma, Glioma-Astrocytoma-Glioblastoma, Glioma-Oligoastrocytoma, Glioma-Oligodendroglioma), Breast cancer (e.g. ADC, ADC-Ductal, ADC-Ductal-TNBC, ADC-Ductal-TPBC, ADC-Lobular), Colorectal cancer (e.g. ADC), Esophageal cancer (e.g. ADC), Esophageal cancer (e.g. SCC), Gastric cancer (e.g. ADC, ADC-Diffuse, ADC-Intestinal, ADC-Intestinal-Tubular), Head and Neck cancer (e.g. Laryngeal cancer-SCC, SCC, Oral cancer-SCC), Kidney cancer (e.g. ccRCC, Chromophobe, Papillary, Papillary-Type I, Papillary-Type II), Liver cancer (e.g. HCC), Lung cancer (e.g. NSCLC-ADC, NSCLC-ADC-Mixed, NSCLC-SCC, SCLC), Mesothelioma (e.g. Epithelioid), Ovarian cancer (e.g. ADC-Cystadenocarcinoma-Papillary serous), Pancreatic cancer (e.g. ADC-Ductal), Prostate cancer (e.g. ADC-Acinar type), Sarcoma (e.g. Leiomyosarcoma, Liposarcoma-Dedifferentiated, Malignant fibrous histiocytoma), Skin cancer (e.g. Melanoma), Testicular cancer (e.g. Germ cell tumor-Seminoma), Thymoma, Thyroid cancer (e.g. Follicular carcinoma, Papillary carcinoma-Classical variant), or Uterine cancer (e.g. Cervical-SCC, Cervical-SCC-Keratinizing, Cervical-SCC-Non-keratinizing, Endometrial-ADC-Endometrioid, Endometrial-ADC-Papillary serous, Endometrial-Carcinosarcoma-Malignant mixed mullerian tumor), B cell lymphoma and T cell lymphoma (cf. Table 11.1.2).

In some embodiments, the tumor is selected from T-cell acute lymphoblastic leukemia, breast cancer, triple-negative breast cancer, triple positive breast cancer, non-small cell lung cancer (NSCLC), small cell lung cancer (SCLC), testicular cancer, gastric cancer, head and neck squamous cell carcinoma, thymoma, esophageal adenocarcinoma, colorectal cancer, pancreatic adenocarcinoma, ovarian cancer or cervical cancer, acute myeloid leukemia, kidney cancer, bladder cancer, skin cancer, skin cancer, melanoma, thyroid cancer, mesothelioma, sarcoma and prostate cancer or any other cancer involving chemokine receptor or CCR8 expressing cells. Preferably the tumor is selected from head and neck cancer, breast cancer, gastric cancer, lung cancer, squamous cell carcinoma, esophageal tumor, melanoma, bladder cancer, liver cancer, and/or prostate cancer.

In some embodiments, the cancer is a solid cancer selected from squamous cell carcinoma, small cell lung cancer (SCLC), non-small cell lung cancer (NSCLC), squamous NSCLC, non-squamous NSCLC, head and neck cancer, breast cancer, cancer of the esophagus, gastric cancer, gastrointestinal cancer, cancer of the small intestine, liver cancer, hepatocellular carcinoma (HCC), pancreatic cancer (PAC), kidney cancer, renal cell carcinoma (RCC), bladder cancer, cancer of the urethra, cancer of the ureter, colorectal cancer (CRC), colon cancer, colon carcinoma, cancer of the anal region, endometrial cancer, prostate cancer, a fibrosarcoma, neuroblastoma, glioma, glioblastoma, germ cell tumor, pediatric sarcoma, sinonasal natural killer, melanoma, skin cancer, bone cancer, cervical cancer, uterine cancer, carcinoma of the endometrium, carcinoma of the fallopian tubes, ovarian cancer, carcinoma of the cervix, carcinoma of the vagina, carcinoma of the vulva, testicular cancer, cancer of the endocrine system, thyroid cancer, cancer of the parathyroid gland, cancer of the adrenal gland, sarcoma of soft tissue, cancer of the penis, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, solid tumors of childhood, environmentally-induced cancers, virus related cancers, cancers of viral origin, advanced cancer, unresectable cancer, metastatic cancer, refractory cancer, recurrent cancer, and any combination thereof.

### C. Combination Treatments

Provided herein are combination treatments or co-treatments for use in systems, compositions, and/or methods of treatment as described herein. In some embodiments, co-treatments encompassed by the present disclosure can be administered simultaneously, separately, or in sequential combination with one or more further therapeutically active compounds, including anti-CCR8 binding agents provided herein. Any suitable co-treatments can be used in the systems, compositions, and/or methods of the present disclosure, and which include co-treatments for the treatment of cancer. In some embodiments, combination treatments comprise checkpoint inhibits, chemokine receptor antibodies, HER2 or EGFR Targeting antibodies or molecules, therapeutic antibodies, and cytotoxic or cytostatic agents.

In some embodiments, co-treatments comprise an antibody or a small molecule targeting a checkpoint protein, such as PD1, PD-L1 or CTLA-4. Suitable checkpoint targeting antibodies include Nivolumab (PD1; Human IgG4), Pembrolizumab (PD1; Humanized IgG4), Atezolizumab (PD-L1; Humanized IgG1), Avelumab (PD-L1; Human IgG1), Durvalumab (PD-L1; Human IgG1), Cemiplimab, cemiplimab-rwlc (PD-1; Human mAb), Dostarlimab (TSR-042) (PD-1; Humanized IgG4), or Ipilimumab (CTLA-4; Human IgG1).

In some embodiments, the antibody or a small molecule targeting a checkpoint protein targets CTLA-4, PDL1, PDL2, PD1, B7-H3, B7-H4, BTLA, HVEM, TIM3, GALS, LAG3, VISTA, KIR, 2B4, CD160, CGEN-15049, CHK 1, CHK2, A2aR, B-7 family ligands or a combination thereof.

In some embodiments, co-treatments comprise an antibody or a small molecule targeting a further chemokine receptor, such as CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1 or CXCR1. Suitable antibodies targeting a further chemokine receptor include the antibodies provided herein, or Mogamulizumab.

In some embodiments, co-treatments comprise an antibody or a small molecule targeting HER2 and/or EGFR. Suitable antibodies targeting HER2 are Trastuzumab (HER2; Humanized IgG1), Pertuzumab (HER2; humanized IgG1), Ado-trastuzumab emtansine (HER2; humanized IgG1; ADC), [fam-]trastuzumab deruxtecan, fam-trastuzumab deruxtecan-nxki (HER2; Humanized IgG1 ADC), Sacituzumab govitecan; sacituzumab govitecan-hziy (TROP-2; Humanized IgG1 ADC) and/or Margetuximab (HER2; Chimeric IgG1). Suitable antibodies targeting EGFR are Cetuximab (EGFR; Chimeric IgG1), Panitumumab (EGFR; Human IgG2), and Necitumumab (EGFR; Human IgG1).

In some embodiments, co-treatments comprise a further therapeutic antibody selected from Muromonab-CD3 (CD3; Murine IgG2a), Efalizumab (CD11a; Humanized IgG1), Tositumomab-I131 (CD20; Murine IgG2a), Nebacumab (Endotoxin; Human IgM), Edrecolomab (EpCAM; Murine IgG2a), Catumaxomab (EPCAM/CD3; Rat/mouse bispecific mAb), Daclizumab (IL-2R; Humanized IgG1), Abciximab (GPIIb/IIIa; Chimeric IgG1 Fab), Rituximab (CD20; Chimeric IgG1), Basiliximab (IL-2R; Chimeric IgG1), Palivizumab (RSV; Humanized IgG1), Infliximab (TNF; Chimeric IgG1), Trastuzumab (HER2; Humanized IgG1), Adalimumab (TNF; Human IgG1), Ibritumomab tiuxetan (CD20; Murine IgG1), Omalizumab (IgE; Humanized IgG1), Cetuximab (EGFR; Chimeric IgG1), Bevacizumab (VEGF; Humanized IgG1), Natalizumab (a4 integrin; Humanized IgG4), Panitumumab (EGFR; Human IgG2), Ranibizumab (VEGF; Humanized IgG1 Fab), Eculizumab (C5; Humanized IgG2/4), Certolizumab pegol (TNF; Humanized Fab, pegylated), Ustekinumab (IL-12/23; Human IgG1), Canakinumab (IL-1; Human IgG1), Golimumab (TNF; Human IgG1), Ofatumumab (CD20; Human IgG1), Tocilizumab (IL-6R; Humanized IgG1), Denosumab (RANK-L; Human IgG2), Belimumab (BLyS; Human IgG1), Ipilimumab (CTLA-4; Human IgG1), Brentuximab vedotin (CD30; Chimeric IgG1; ADC), Pertuzumab (HER2; humanized IgG1), Ado-trastuzumab emtansine (HER2; humanized IgG1; ADC), Raxibacumab (B. anthrasis PA; Human IgG1), Obinutuzumab (CD20; Humanized IgG1 Glycoengineered), Siltuximab (IL-6; Chimeric IgG1), Ramucirumab (VEGFR2; Human IgG1), Vedolizumab (a4137 integrin; humanized IgG1), Nivolumab (PD1; Human IgG4), Pembrolizumab (PD1; Humanized IgG4), Blinatumomab (CD19, CD3; Murine bispecific tandem scFv), Alemtuzumab (CD52; Humanized IgG1), Evolocumab (PCSK9; Human IgG2), Idarucizumab (Dabigatran; Humanized Fab), Necitumumab (EGFR; Human IgG1), Dinutuximab (GD2; Chimeric IgG1), Secukinumab (IL-17a; Human IgG1), Mepolizumab (IL-5; Humanized IgG1), Alirocumab (PCSK9; Human IgG1), Daratumumab (CD38; Human IgG1), Elotuzumab (SLAMF7; Humanized IgG1), Ixekizumab (IL-17a; Humanized IgG4), Reslizumab (IL-5; Humanized IgG4), Olaratumab (PDGFRa; Human IgG1), Bezlotoxumab (Clostridium difficile enterotoxin B; Human IgG1), Atezolizumab (PD-L1; Humanized IgG1), Obiltoxaximab (B. anthrasis PA; Chimeric IgG1), Brodalumab (IL-17R; Human IgG2), Dupilumab (IL-4R a; Human IgG4), Inotuzumab ozogamicin (CD22; Humanized IgG4; ADC), Guselkumab (IL-23 p19; Human IgG1), Sarilumab (IL-6R; Human IgG1), Avelumab (PD-L1; Human IgG1), Emicizumab (Factor Ixa, X; Humanized IgG4, bispecific), Ocrelizumab (CD20; Humanized IgG1), Benralizumab (IL-5R a; Humanized IgG1), Durvalumab (PD-L1; Human IgG1), Gemtuzumab ozogamicin (CD33; Humanized IgG4; ADC), Erenumab, erenumab-aooe (CGRP receptor; Human IgG2), Galcanezumab, galcanezumab-gnlm (CGRP; Humanized IgG4), Burosumab, burosumab-twza (FGF23; Human IgG1), Lanadelumab, lanadelumab-flyo (Plasma kallikrelin; Human IgG1), Mogamulizumab, mogamulizumab-kpkc (CCR4; Humanized IgG1), Tildrakizumab; tildrakizumab-asmn (IL-23 p19; Humanized IgG1), Fremanezumab, fremanezumab-vfrm (CGRP; Humanized IgG2), Ravulizumab, ravulizumab-cwvz (C5; Humanized IgG2/4), Cemiplimab, cemiplimab-rwlc (PD-1; Human mAb), Ibalizumab, ibalizumab-uiyk (CD4; Humanized IgG4), Emapalumab, emapalumab-lzsg (IFNg; Human IgG1), Moxetumomab pasudotox, moxetumomab pasudotox-tdfk (CD22; Murine IgG1 dsFv immunotoxin), Caplacizumab, caplacizumab-yhdp (von Willebrand factor; Humanized Nanobody), Risankizumab, risankizumab-rzaa (IL-23 p 19; Humanized IgG1), Polatuzumab vedotin, polatuzumab vedotin-piiq (CD79b; Humanized IgG1 ADC), Romosozumab, romosozumab-aqqg (Sclerostin; Humanized IgG2), Brolucizumab, brolucizumab-dbll (VEGF-A; Humanized scFv), Crizanlizumab; crizanlizumab-tmca (CD62 (aka P-selectin); Humanized IgG2), Enfortumab vedotin, enfortumab vedotin-ejfv (Nectin-4; Human IgG1 ADC), [fam-]trastuzumab deruxtecan, fam-trastuzumab deruxtecan-nxki (HER2; Humanized IgG1 ADC), Teprotumumab, teprotumumab-trbw (IGF-1R; Human IgG1), Eptinezumab, eptinezumab-jjmr (CGRP; Humanized IgG1), Isatuximab, isatuximab-irfc (CD38; Chimeric IgG1), Sacituzumab govitecan; sacituzumab govitecan-hziy (TROP-2; Humanized IgG1 ADC), Inebilizumab (CD19; Humanized IgG1), Leronlimab (CCR5; Humanized IgG4), Satralizumab (IL-6R; Humanized IgG2), Narsoplimab (MASP-2, Human IgG4), Tafasitamab (CD19; Humanized IgG1), REGNEB3 (Ebola virus; mixture of 3 human IgG1), Naxitamab (GD2; Humanized IgG1), Oportuzumab monatox (EpCAM; Humanized scFv immunotoxin), Belantamab mafodotin (B-cell maturation antigen; Humanized IgG1 ADC), Margetuximab (HER2; Chimeric IgG1), Tanezumab (Nerve growth factor; Humanized IgG2), Dostarlimab (TSR-042) (PD-1; Humanized IgG4), Teplizumab (CD3; Humanized IgG1), Aducanumab (Amyloid beta; Human IgG1), Sutimlimab (BIVV009) (Cis; Humanized IgG4), Evinacumab (Angiopoietin-like 3; Human IgG4).

In some embodiments, co-treatments comprise a cytostatic and/or cytotoxic agent selected from radionuclides, alkylating agents, DNA cross-linking agents, DNA intercalating agents (e.g., groove binding agents such as minor groove binders), cell cycle modulators, apoptosis regulators, kinase inhibitors, protein synthesis inhibitors, mitochondria inhibitors, nuclear export inhibitors, topoisomerase I inhibitors, topoisomerase II inhibitors, RNA/DNA antimetabolites and antimitotic agents.

In some embodiments, the cytotoxic agent is an auristatin, a maytansinoid, a kinesin-spindle protein (KSP) inhibitor, a nicotinamide phosphoribosyltransferase (NAMPT) inhibitor or a pyrrolobenzodiazepine derivative.

### SEQUENCES

The sequences in following TABLE 1 are exemplary sequences of CCR8 and epitopes thereof as described herein.

TABLE 1
Exemplary amino acid sequences of CCR8

| SEQ | | |
|---|---|---|
| ID | | |
| NO: | Description | Amino Acid Sequence |
| 1 | Human CCR8 | MDYTLDLSVTTVTDYYYPDIFSSPCDAELIQTNGKLLLAVFYCLLFVFSLLGNSLVILVLVVCKKLRSITDVYL |
| | (UniProt | LNLALSDLLFVFSFPFQTYYLLDQWVFGTVMCKVVSGFYYIGFYSSMFFITLMSVDRYLAVVHAVYALKVRTIRM |
| | Identifier | GTTLCLAVWLTAIMATIPLLVFYQVASEDGVLQCYSFYNQQTLKWKIFTNFKMNILGLLIPFTIFMFCYIKILHQ |
| | P51685) | LKRCQNHNKTKAIRLVLIVVIASLLFWVPFNVVLFLTSLHSMHILDGCSISQQLTYATHVTEIISFTHCCVNPVI |
| | | YAFVGEKFKKHLSEIFQKSCSQIFNYLGRQMPRESCEKSSSCQQHSSRSSSVDYIL |
| 2 | Mouse CCR8 | MDYTMEPNVTMTDYYPDFFTAPCDAEFLLRGSMLYLAILYCVLFVLGLLGNSLVILVLVGCKKLRSITDIYLLNL |
| | (UniProt | AASDLLFVLSIPFQTHNLLDQWVFGTAMCKVVSGLYYIGFFSSMFFITLMSVDRYLAIVHAVYAIKVRTASVGTA |
| | Identifier | LSLTVWLAAVTATIPLMVFYQVASEDGMLQCFQFYEEQSLRWKLFTHFEINALGLLLPFAILLFCYVRILQQLRG |
| | P56484) | CLNHNRTRAIKLVLTVVIVSLLFWVPFNVALFLTSLHDLHILDGCATRQRLALAIHVTEVISFTHCCVNPVIYAF |
| | | IGEKFKKHLMDVFQKSCSHIFLYLGRQMPVGALERQLSSNQRSSHSSTLDDIL |
| 3 | Monkey CCR8 | MDYTLDPSMTTMTDYYYPDSLSSPCDGELIQRNDKLLLAVFYCLLFVFSLLGNSLVILVLVVCKKLRNITDIYLL |
| | (UniProt | NLALSDLLFVFSFPFQTYYQLDQWVFGTVMCKVVSGFYYIGFYSSMFFITLMSVDRYLAVVHAVYAIKVRTIRM |
| | Identifier | GTTTLSLLVWLTAIMATIPLLVFYQVASEDGVLQCYSFYNQQTLKWKIFTNFEMNILGLLIPFTIFMFCYIKILH |
| | O97665) | QLKRCQNHNKTKAIRLVLIVVIASLLFWVPFNVVLFLTSLHSMHILDGCSISQQLNYATHVTEIISFTHCCVNPV |
| | | IYAFVGEKFKKHLSEIFQKSCSHIFIYLGRQMPRESCEKSSSCQQHSFRSSSIDYIL |
| 4 | Rabbit CCR8 | MDYTLEPNVTAVTDYYYPDIFSSPCDGELTQRRSKLPLAIFYCLLFVSGLLGNGLVILVLVAYKKLQSITDIYLL |
| | (Uniprot | NLALSDLLFVFSFPFLTHYQLDQWVFGTPMCKLVSGLYYVGFFSNMFFITLMSVDRYLAVVHAVCALRVRTART |
| | Identifier | GVALSLAVWLTAIMATSPLLVFYQVVPEDGVLQCYLSYNQQTLRWKIFTHLEMNVLGLLIPFAVLMFCYVRILQ |
| | G1T2B2) | QLRHCQNHNKTKAIKLVLIVVLASLLFWVPFNVVLFLTSLHNMHVLDGCVINQRLIYATHVTEAISFTHCCVNP |
| | | VIYAFMGEKFKKYLSDIFQKSCSHVVFYIGRNVSRQGYERSSSFPPSSSRSSIMDHIL |
| 15 | C-terminal | CQQHSSRSSSVDYIL |
| | Intracellular | |
| | Epitope | |
| 16 | Extracellular | MDYTLDLSVTTVTDYYYPDIFSSPCDAELIQTNGK |
| | sequence(s) | |
| 17 | C-terminal | CSQIFNYLGRQMPRES |
| | Intracellular | |
| | Epitope | |

The sequences in following TABLE 1.1 are exemplary sequences of CCR1-7,9 and epitopes thereof as described herein.

TABLE 1.1
Exemplary amino acid sequences of CCR1-7,9

| SEQ | | |
|---|---|---|
| ID | | |
| NO: | Description | Amino Acid Sequence |
| 18 | Human CCR1 | METPNTTEDYDTTTEFDYGDATPCQKVNERAFGAQLLPPLYSLVFVIGLVGNILVVLVLVQYKRLKNMTSIYLL |
| | (UniProt | NLAISDLLFLFTLPFWIDYKLKDDWVFGDAMCKILSGFYYTGLYSEIFFIILLTIDRYLAIVHAVFALRARTVTFGV |
| | Identifier | ITSIIIWALAILASMPGLYFSKTQWEFTHHTCSLHFPHESLREWKLFQALKLNLFGLVLPLLVMIICYTGIIKILLRR |
| | P32246) | PNEKKSKAVRLIFVIMIIFFLFWTPYNLTILISVFQDFLFTHECEQSRHLDLAVQVTEVIAYTHCCVNPVIYAFVGE |
| | | RFRKYLRQLFHRRVAVHLVKWLPFLSVDRLERVSSTSPSTGEHELSAGF |
| 19 | Human CCR2 | MLSTSRSRFIRNTNESGEEVTTFFDYDYGAPCHKFDVKQIGAQLLPPLYSLVFIFGFVGNMLVVLILINCKKLKCL |
| | (UniProt | TDIYLLNLAISDLLFLITLPLWAHSAANEWVFGNAMCKLFTGLYHIGYFGGIFFIILLTIDRYLAIVHAVFALKART |
| | Identifier | VTFGVVTSVITWLVAVFASVPGIIFTKCQKEDSVYVCGPYFPRGWNNFHTIMRNILGLVLPLLIMVICYSGILKTL |
| | P41597) | LRCRNEKKRHRAVRVIFTIMIVYFLFWTPYNIVILLNTFQEFFGLSNCESTSQLDQATQVTETLGMTHCCINPIIYA |
| | | FVGEKFRSLFHIALGCRIAPLQKPVCGGPGVRPGKNVKVTTQGLLDGRGKGKSIGRAPEASLQDKEGA |
| 20 | Human CCR3 | MTTSLDTVETFGTTSYYDDVGLLCEKADTRALMAQFVPPLYSLVFTVGLLGNVVVVMILIKYRRLRIMTNIYLL |
| | (UniProt | NLAISDLLFLVTLPFWIHYVRGHNWVFGHGMCKLLSGFYHTGLYSEIFFIILLTIDRYLAIVHAVFALRARTVTFG |
| | Identifier | VITSIVTWGLAVLAALPEFIFYETEELFEETLCSALYPEDTVYSWRHFHTLRMTIFCLVLPLLVMAICYTGIIKTLL |
| | P51677) | RCPSKKKYKAIRLIFVIMAVFFIFWTPYNVAILLSSYQSILFGNDCERSKHLDLVMLVTEVIAYSHCCMNPVIYAF |
| | | VGERFRKYLRHFFHRHLLMHLGRYIPFLPSEKLERTSSVSPSTAEPELSIVF |
| 21 | Human CCR4 | MNPTDIADTTLDESIYSNYYLYESIPKPCTKEGIKAFGELFLPPLYSLVFVFGLLGNSVVVLVLFKYKRLRSMTDV |
| | (UniProt | YLLNLAISDLLFVFSLPFWGYYAADQWVFGLGLCKMISWMYLVGFYSGIFFVMLMSIDRYLAIVHAVESLRART |
| | Identifier | LTYGVITSLATWSVAVFASLPGFLFSTCYTERNHTYCKTKYSLNSTTWKVLSSLEINILGLVIPLGIMLFCYSMIIR |
| | P51679) | TLQHCKNEKKNKAVKMIFAVVVLFLGFWTPYNIVLFLETLVELEVLQDCTFERYLDYAIQATETLAFVHCCLNPI |
| | | IYFFLGEKFRKYILQLFKTCRGLFVLCQYCGLLQIYSADTPSSSYTQSTMDHDLHDAL |
| 22 | Human CCR5 | MDYQVSSPIYDINYYTSEPCQKINVKQIAARLLPPLYSLVFIFGFVGNMLVILILINCKRLKSMTDIYLLNLAISDLF |
| | (UniProt | FLLTVPFWAHYAAAQWDFGNTMCQLLTGLYFIGFFSGIFFIILLTIDRYLAVVHAVFALKARTVTFGVVTSVITW |
| | Identifier | VVAVFASLPGIIFTRSQKEGLHYTCSSHFPYSQYQFWKNFQTLKIVILGLVLPLLVMVICYSGILKTLLRCRNEKK |
| | P51681) | RHRAVRLIFTIMIVYFLFWAPYNIVLLLNTFQEFFGLNNCSSSNRLDQAMQVTETLGMTHCCINPIIYAFVGEKFR |
| | | NYLLVFFQKHIAKRFCKCCSIFQQEAPERASSVYTRSTGEQEISVGL |
| 23 | Human CCR6 | MSGESMNFSDVFDSSEDYFVSVNTSYYSVDSEMLLCSLQEVRQFSRLFVPIAYSLICVFGLLGNILVVITFAFYKK |
| | (UniProt | ARSMTDVYLLNMAIADILFVLTLPFWAVSHATGAWVFSNATCKLLKGIYAINFNCGMLLLTCISMDRYIAIVQA |
| | Identifier | TKSFRLRSRTLPRSKIICLVVWGLSVIISSSTFVFNQKYNTQGSDVCEPKYQTVSEPIRWKLLMLGLELLFGFFIPL |
| | P51684) | MFMIFCYTFIVKTLVQAQNSKRHKAIRVIIAVVLVFLACQIPHNMVLLVTAANLGKMNRSCQSEKLIGYTKTVTE |
| | | VLAFLHCCLNPVLYAFIGQKFRNYFLKILKDLWCVRRKYKSSGFSCAGRYSENISRQTSETADNDNASSFTM |
| 24 | Human CCR7 | MDLGKPMKSVLVVALLVIFQVCLCQDEVTDDYIGDNTTVDYTLFESLCSKKDVRNFKAWFLPIMYSIICFVGLL |
| | (UniProt | GNGLVVLTYIYFKRLKTMTDTYLLNLAVADILFLLTLPFWAYSAAKSWVFGVHFCKLIFAIYKMSFFSGMLLLL |
| | Identifier | CISIDRYVAIVQAVSAHRHRARVLLISKLSCVGIWILATVLSIPELLYSDLQRSSSEQAMRCSLITEHVEAFITIQVA |
| | P32248) | QMVIGFLVPLLAMSFCYLVIIRTLLQARNFERNKAIKVIIAVVVVFIVFQLPYNGVVLAQTVANFNITSSTCELSKQ |
| | | LNIAYDVTYSLACVRCCVNPFLYAFIGVKFRNDLFKLFKDLGCLSQEQLRQWSSCRHIRRSSMSVEAETTTTFSP |
| 25 | Human CCR9 | MTPTDFTSPIPNMADDYGSESTSSMEDYVNFNFTDFYCEKNNVRQFASHFLPPLYWLVFIVGALGNSLVILVYW |
| | (UniProt | YCTRVKTMTDMFLLNLAIADLLFLVTLPFWAIAAADQWKFQTFMCKVVNSMYKMNFYSCVLLIMCISVDRYIA |
| | Identifier | IAQAMRAHTWREKRLLYSKMVCFTIWVLAAALCIPEILYSQIKEESGIAICTMVYPSDESTKLKSAVLTLKVILGF |
| | P51686) | FLPFVVMACCYTIIIHTLIQAKKSSKHKALKVTITVLTVFVLSQFPYNCILLVQTIDAYAMFISNCAVSTNIDICFQV |
| | | TQTIAFFHSCLNPVLYVFVGERFRRDLVKTLKNLGCISQAQWVSFTRREGSLKLSSMLLETTSGALSL |

The sequences in the following TABLE 2 illustrate exemplary CDR amino acid sequences that can be used to generate the polypeptide sequences of the present disclosure (e.g., anti-CCR8 agents) and/or systems and compositions and perform the methods described herein. The exemplary CDR amino acid sequences depicted in TABLE 2 are found in Clone 433H (BD 566379 and BD 624084).

TABLE 2
EXEMPLARY AMINO ACID SEQUENCES

| | Descri- | | | | | | |
|---|---|---|---|---|---|---|---|
| AB | ption | CDRH1 | CDRH2 | CDRH3 | CDRL1 | CDRL2 | CDRL3 |
| AB-1 | Kabat | TFAMN | RIRSKSNNYATY | GKENNYGYAMD | RSSQSLLHSNGN | RMSNLAS | MQHLEYPFT |
| | | (SEQ ID | YADSV | Y | TYLY | (SEQ ID | (SEQ ID |
| | | NO: 5) | (SEQ ID | (SEQ ID | (SEQ ID | NO: 9) | NO: 10) |
| | | | NO: 6) | NO: 7) | NO: 8) | | |
| | Chothia | GFPFNTFAMN | RSKSNNYA | GKENNYGYAMD | RSSQSLLHSNGN | RMSNLAS | MQHLEYPFT |
| | | (SEQ ID | (SEQ ID | Y | TYLY | (SEQ ID | (SEQ ID |
| | | NO: 63) | NO: 64) | (SEQ ID | (SEQ ID | NO: 67) | NO: 68) |
| | | | | NO: 65) | NO: 66) | | |
| | IMGT | GFPFNTFA | IRSKSNNYAT | VRGKENNYGYA | RSSQSLLHSNGN | RMSNLAS | MQHLEYPFT |
| | | (SEQ ID | (SEQ ID | MDY | TYLY | (SEQ ID | (SEQ ID |
| | | NO: 69) | NO: 70) | (SEQ ID | (SEQ ID | NO: 73) | NO: 26) |
| | | | | NO: 71) | NO: 72) | | |
| | North | AASGFPFNTF | RIRSKSNNYATY | VRGKENNYGYA | RSSQSLLHSNGN | RMSNLAS | MQHLEYPFT |
| | | AMN | (SEQ ID | MDY | TY | (SEQ ID | (SEQ ID |
| | | (SEQ ID | NO: 28) | (SEQ ID | (SEQ ID | NO: 31) | NO: 32) |
| | | NO: 27) | | NO: 29) | NO: 30) | | |
| | CCG | GFPFNTFAMN | RIRSKSNNYATY | GKENNYGYAMD | RSSQSLLHSNGN | RMSNLAS | MQHLEYPFT |
| | | (SEQ ID | YADSVKD | Y | TY | (SEQ ID | (SEQ ID |
| | | NO: 33) | (SEQ ID | (SEQ ID | (SEQ ID | NO: 37) | NO: 38) |
| | | | NO: 34) | NO: 35) | NO: 36) | | |

The sequences in the following TABLE 3 illustrate exemplary VH and VL amino acid sequences that can be used to generate the polypeptide sequences of the present disclosure (e.g., anti-CCR8 agents) and/or systems and compositions and perform the methods described herein. The exemplary VH and VL amino acid sequences depicted in TABLE 3 are found in Clone 433H (BD 566379 and BD 624084).

TABLE 3
EXEMPLARY VH AND VL AMINO ACID SEQUENCES

| AB | VH | VL |
|---|---|---|
| AB-1 | EVQLVETGGGLVQPKGSLRLSCAASGFPFNTFAMNW | DIVMTQAAPSVPVTPGESVSISCRSSQSLLHSNGNTYLYWFLQ |
| | VRQAPGKGLEWLARIRSKSNNYATYYADSVKDRFTI | RPGQSPQLLIYRMSNLASGVPDRESGSGSGTAFTLRISRVEAED |
| | SRDDSRRMLYLLMNNVKFEDTAIYYCVRGKENNYGY | VGVYYCMQHLEYPFTFGIGTKLELK |
| | AMDYWGQGTSVTVSS (SEQ ID NO: 11) | (SEQ ID NO: 12) |

The sequences in the following TABLE 4 illustrate exemplary HC and LC amino acid sequences that can be used to generate the polypeptide sequences of the present disclosure (e.g., anti-CCR8 agents) and/or systems and compositions and perform the methods described herein. The exemplary HC and LC amino acid sequences depicted in TABLE 4 are found in Clone 433H (BD 566379 and BD 624084).

TABLE 4
EXEMPLARY HC and LC AMINO ACID SEQUENCES

| AB | HC | LC |
|---|---|---|
| AB-1 | EVQLVETGGGLVQPKGSLRLSCAASGFPFNTFAMNWVRQAPGKGLE | DIVMTQAAPSVPVTPGESVSISCRSSQSLLHS |
| | WLARIRSKSNNYATYYADSVKDRFTISRDDSRRMLYLLMNNVKFED | NGNTYLYWFLQRPGQSPQLLIYRMSNLASG |
| | TAIYYCVRGKENNYGYAMDYWGQGTSVTVSSASTKGPSVFPLAPSS | VPDRFSGSGSGTAFTLRISRVEAEDVGVYYC |
| | KSTSGGTAALGCLVKDYFPEPVTVSWNSGALTSGVHTFPAVLQSSG | MQHLEYPFTFGIGTKLELKRTVAAPSVFIFPP |
| | LYSLSSVVTVPSSSLGTQTYICNVNHKPSNTKVDKKVEPKSCDKTH | SDEQLKSGTASVVCLLNNFYPREAKVQWKV |
| | TCPPCPAPELLGGPSVFLFPPKPKDTLMISRTPEVTCVVVDVSHED | DNALQSGNSQESVTEQDSKDSTYSLSSTLTLS |
| | PEVKFNWYVDGVEVHNAKTKPREEQYNSTYRVVSVLTVLHQDWLNG | KADYEKHKVYACEVTHQGLSSPVTKSFNRG |
| | KEYKCKVSNKALPAPIEKTISKAKGQPREPQVYTLPPSREEMTKNQ | EC |
| | VSLTCLVKGFYPSDIAVEWESNGQPENNYKTTPPVLDSDGSFFLYS | (SEQ ID NO: 14) |
| | KLTVDKSRWQQGNVFSCSVMHEALHNHYTQKSLSLSPGK (SEQ | |
| | ID NO: 13) | |

The sequences in the following TABLE 5 illustrate exemplary HC and LC amino acid sequences of certain therapeutic anti-CCR8 antibodies (tABs) that bind to CCR8 and can be used in the methods provided herein.

TABLE 5
EXEMPLARY HC AND LC AMINO ACID SEQUENCES OF
THERAPEUTIC ANTI-CCR8 ANTIBODIES

| AB | HC | LC |
|---|---|---|
| tAB-1 | EVQLVETGGGLVQPKGSLRLSCAASGFPFNTFAM | DIVMTQAAPSVPVTPGESVS |
| denikitug, | NWVRQAPGKGLEWLARIRSKSNNYATYYADSVK | ISCRSSQSLLHSNGNTYLYW |
| GS- | DRFTISRDDSRRMLYLLMNNVKFEDTAIYYCVRG | FLQRPGQSPQLLIYRMSNLA |
| 1811) | KENNYGYAMDYWGQGTSVTVSSAKTTAPSVYPL | SGVPDRFSGSGSGTAFTLRIS |
| | APVCGDTTGSSVTLGCLVKGYFPEPVTLTWNSGSL | RVEAEDVGVYYCMQHLEY |
| | SSGVHTFPAVLQSDLYTLSSSVTVTSSTWPSQSITC | PFTFGIGTKLELKRADAAPT |
| | NVAHPASSTKVDKKIEPRGPTIKPCPPCKCPAPNLL | VSIFPPSSEQLTSGGASVVCF |
| | GGPSVFIFPPKIKDVLMISLSPIVTCVVVDVSEDDPD | LNNFYPKDINVKWKIDGSER |
| | VQISWFVNNVEVHTAQTQTHREDYNSTLRVVSAL | QNGVLNSWTDQDSKDSTYS |
| | PIQHQDWMSGKEFKCKVNNKDLPAPIERTISKPKG | MSSTLTLTKDEYERHNSYTC |
| | SVRAPQVYVLPPPEEEMTKKQVTLTCMVTDFMPE | EATHKTSTSPIVKSFNRNEC |
| | DIYVEWTNNGKTELNYKNTEPVLDSDGSYFMYSK | (SEQ ID NO: 40) |
| | LRVEKKNWVERNSYSCSVVHEGLHNHHTTKSFSR | |
| | TPG | |
| | (SEQ ID NO: 39) | |
| tAB-2 | EVQLVESGGGLVQPGGSLKLSCAASGFIFSNAVMY | ETVVTQSPATLSLSPGERAT |
| (azirktug,) | WVRQASGKGLEWVAIKTKFNNYATYYADAVKGR | LSCRASTSVITLLHWFQQKP |
| | FTISRDDSKNMVYLQMNSLKTEDTAVYYCTAGDR | GQAPRLLIHGASNLESRVPA |
| | NKPFAYWGQGTLVTVSSASTKGPSVFPLAPSSKST | RFSGSGSGTDFTLTISSLEPE |
| | SGGTAALGCLVKDYFPEPVTVSWNSGALTSGVHT | DFATYFCQQSWNDPYTFGQ |
| | FPAVLQSSGLYSLSSVVTVPSSSLGTQTYICNVNHK | GTKLEIKRTVAAPSVFIFPPS |
| | PSNTKVDKKVEPKSCDKTHTCPPCPAPELLGGPSV | DEQLKSGTASVVCLLNNFY |
| | FLFPPKPKDTLMISRTPEVTCVVVDVSHEDPEVKF | PREAKVQWKVDNALQSGN |
| | NWYVDGVEVHNAKTKPREEQYNSTYRVVSVLTV | SQESVTEQDSKDSTYSLSST |
| | LHQDWLNGKEYKCKVSNKALPAPIEKTISKAKGQ | LTLSKADYEKHKVYACEVT |
| | PREPQVYTLPPSREEMTKNQVSLTCLVKGFYPSDI | HQGLSSPVTKSFNRGEC |
| | AVEWESNGQPENNYKTTPPVLDSDGSFFLYSKLTV | (SEQ ID NO: 42) |
| | DKSRWQQGNVFSCSVMHEALHNHYTQKSLSLSPG | |
| | K | |
| | (SEQ ID NO: 41) | |
| tAB-3 | EVQLVESGGGLVQPGGSLKLSCAASGFTFNTYAM | DIVMTQSPLSLPVTPGEPASI |
| (cafelkibart) | NWVRQASGKGLEWVARIRSKANNYATYYADSVK | SCRSSKSLLHSNANTYLYWF |
| | DRFTISRDDSKNTLYLQMNNLKTEDTAVYYCVRD | LQKPGQSPQLLIYRMSNLAS |
| | RSRGEDYAMDYWGQGTLVTVSSASTKGPSVFPLA | GVPDRFSGSGSGTAFTLKIS |
| | PSSKSTSGGTAALGCLVKDYFPEPVTVSWNSGALT | RVEAEDVGVYYCMQHLEY |
| | SGVHTFPAVLQSSGLYSLSSVVTVPSSSLGTQTYIC | PFTFGQGTKLEIKRTVAAPS |
| | NVNHKPSNTKVDKKVEPKSCDKTHTCPPCPAPELL | VFIFPPSDEQLKSGTASVVCL |
| | GGPDVFLFPPKPKDTLMISRTPEVTCVVVDVSHED | LNNFYPREAKVQWKVDNA |
| | PEVKFNWYVDGVEVHNAKTKPREEQYNSTYRVV | LQSGNSQESVTEQDSKDSTY |
| | SVLTVLHQDWLNGKEYKCKVSNKALPAPEEKTIS | SLSSTLTLSKADYEKHKVYA |
| | KAKGQPREPQVYTLPPSRDELTKNQVSLTCLVKGF | CEVTHQGLSSPVTKSFNRGE |
| | YPSDIAVEWESNGQPENNYKTTPPVLDSDGSFFLY | C |
| | SKLTVDKSRWQQGNVFSCSVMHEALHNHYTQKS | (SEQ ID NO: 44) |
| | LSLSPGK | |
| | (SEQ ID NO: 43) | |
| tAB-4 | EVQLLESGGGLVQPGGSLRLSCAASGFTFSSYGMH | QSVLTQPPSVSGAPGQRVTI |
| (lanerkitug) | WVRQAPGKGLEWVSAINWNGGSTGYADSVKGRF | SCTGSSSNIGAGYNVHWYQ |
| | TISRDNSKNTLYLQMNSLRAEDTAVYYCARGHHS | QLPGTAPKLLIYTNNRRPSG |
| | GYDGRFFDYWGQGTLVTVSSASTKGPSVFPLAPSS | VPDRFSGSKSGTSASLAISGL |
| | KSTSGGTAALGCLVKDYFPEPVTVSWNSGALTSG | RSEDEADYYCAAWDASLSG |
| | VHTFPAVLQSSGLYSLSSVVTVPSSSLGTQTYICNV | WVFGGGTKLTVLGQPKAAP |
| | NHKPSNTKVDKKVEPKSCDKTHTCPPCPAPELLGG | SVTLFPPSSEELQANKATLV |
| | PSVFLFPPKPKDTLMISRTPEVTCVVVDVSHEDPEV | CLISDFYPGAVTVAWKADS |
| | KFNWYVDGVEVHNAKTKPREEQYNSTYRVVSVL | SPVKAGVETTTPSKQSNNK |
| | TVLHQDWLNGKEYKCKVSNKALPAPIEKTISKAK | YAASSYLSLTPEQWKSHRS |
| | GQPREPQVYTLPPSRDELTKNQVSLTCLVKGFYPS | YSCQV |
| | DIAVEWESNGQPENNYKTTPPVLDSDGSFFLYSKL | (SEQ ID NO: 46) |
| | TVDKSRWQQGNVFSCSVMHEALHNHYTQKSLSLS | |
| | PGK | |
| | (SEQ ID NO: 45) | |
| tAB-5 | QVQLVQSGAEVKKPGASVKVSCKASGYTFTDSEM | DIVMTQTPLSLSVTPGQPASI |
| (imzokitug) | HWVRQATGQGLEWMGAIQPETGGTAYNQKFKAR | SCRSSQSLFHSSGNTYLHWY |
| | VTMTRDTSISTAYMELSSLRSEDTAVYYCARRRRN | LQKPGQPPQLLIYKVSNRFS |
| | FDYWGQGTLVTVSSASTKGPSVFPLAPSSKSTSGG | GVPDRFSGSGSGTDFTLKIS |
| | TAALGCLVKDYFPEPVTVSWNSGALTSGVHTFPA | RVEAEDVGVYYCSQSTHVP |
| | VLQSSGLYSLSSVVTVPSSSLGTQTYICNVNHKPSN | FTFGQGTKLEIKRTVAAPSV |
| | TKVDKRVEPKSCDKTHTCPPCPAPELLGGPSVFLFP | FIFPPSDEQLKSGTASVVCLL |
| | PKPKDTLMISRTPEVTCVVVDVSHEDPEVKENWY | NNFYPREAKVQWKVDNAL |
| | VDGVEVHNAKTKPREEQYNSTYRVVSVLTVLHQD | QSGNSQESVTEQDSKDSTYS |
| | WLNGKEYKCKVSNKALPAPIEKTISKAKGQPREPQ | LSSTLTLSKADYEKHKVYA |
| | VYTLPPSREEMTKNQVSLTCLVKGFYPSDIAVEWE | CEVTHQGLSSPVTKSFNRGE |
| | SNGQPENNYKTTPPVLDSDGSFFLYSKLTVDKSRW | C |
| | QQGNVFSCSVMHEALHNHYTQKSLSLSPG | (SEQ ID NO: 48) |
| | (SEQ ID NO: 47) | |
| tAB-6 | EVQLLESGGGLVQPGGSLRLSCAASGIDLSTYAMG | DIQVTQSPSSLSASVGDRVTI |
| | WVRQAPGKGLEWVGLIHRSGRTYYATWAKGRFTI | TCQASENIANALAWYQQKP |
| | SKDSSKNTLYLQMNSLRAEDTAVYYCTRSYPDYS | GKPPKFLIYGASNLASGVPS |
| | ATASIWGQGTTVTVSSASTKGPSVFPLAPSSKSTSG | RFSGSGSGTDFTFTISSLQPE |
| | GTAALGCLVKDYFPEPVTVSWNSGALTSGVHTFP | DIATYYCQQAYYGNSFVEG |
| | AVLQSSGLYSLSSVVTVPSSSLGTQTYICNVNHKPS | TFGGGTKVEIKRTVAAPSVF |
| | NTKVDKKVEPKSCDKTHTCPPCPAPELLGGPSVFL | IFPPSDEQLKSGTASVVCLL |
| | FPPKPKDTLMISRTPEVTCVVVDVSHEDPEVKFNW | NNFYPREAKVQWKVDNAL |
| | YVDGVEVHNAKTKPREEQYNSTYRVVSVLTVLHQ | QSGNSQESVTEQDSKDSTYS |
| | DWLNGKEYKCKVSNKALPAPIEKTISKAKGQPREP | LSSTLTLSKADYEKHKVYA |
| | QVYTLPPSREEMTKNQVSLTCLVKGFYPSDIAVEW | CEVTHQGLSSPVTKSFNRGE |
| | ESNGQPENNYKTTPPVLDSDGSFFLYSKLTVDKSR | C |
| | WQQGNVFSCSVMHEALHNHYTQKSLSLSPGK | (SEQ ID NO: 50) |
| | (SEQ ID NO: 49) | |
| tAB-7 | EVQLLESGGGLVQPGGSLRLSCAASGIDLSTYAMG | DIQVTQSPSSLSASVGDRVTI |
| (enzelk | WVRQAPGKGLEWVGLIHRSGRTYYATWAKGRFTI | TCQASENIANALAWYQQKP |
| itug) | SKDSSKNTLYLQMNSLRAEDTAVYYCTRSYPDYS | GKPPKFLIYGASNLASGVPS |
| | ATASIWGQGTTVTVSSASTKGPSVFPLAPSSKSTSG | RFSGSGSGTDFTFTISSLQPE |
| | GTAALGCLVKDYFPEPVTVSWNSGALTSGVHTFP | DIATYYCQQAYYGNSFVEG |
| | AVLQSSGLYSLSSVVTVPSSSLGTQTYICNVNHKPS | TFGGGTKVEIKRTVAAPSVF |
| | NTKVDKKVEPKSCDKTHTCPPCPAPELLGGPSVFL | IFPPSDEQLKSGTASVVCLL |
| | FPPKPKDTLMISRTPEVTCVVVDVSHEDPEVKFNW | NNFYPREAKVQWKVDNAL |
| | YVDGVEVHNAKTKPREEQYNSTYRVVSVLTVLHQ | QSGNSQESVTEQDSKDSTYS |
| | DWLNGKEYKCKVSNKALPAPIEKTISKAKGQPREP | LSSTLTLSKADYEKHKVYA |
| | QVYTLPPSREEMTKNQVSLTCLVKGFYPSDIAVEW | CEVTHQGLSSPVTKSFNRGE |
| | ESNGQPENNYKTTPPVLDSDGSFFLYSKLTVDKSR | C |
| | WQQGNVFSCSVMHEALHNHYTQKSLSLSPGK | (SEQ ID NO: 52) |
| | (SEQ ID NO: 51) | |

The sequences in the following TABLE 6 illustrate exemplary acid sequences of an exemplary therapeutic anti-CCR8 antibody that binds to CCR8 and can be used in the methods provided herein.

TABLE 6
EXEMPLARY ANTI-CCR8 ANTIBODY SEQUENCES.

| | | SEQ ID |
|---|---|---|
| Description | Sequence | NO: |
| Anti-CCR8 | GFTFNTYAMN | 53 |
| antibody | | |
| HCDR1 | | |
| Anti-CCR8 | RIRSKSNNYATYYADSVKD | 54 |
| antibody | | |
| HCDR2 | | |
| Anti-CCR8 | VRGLLRYRFFDV | 55 |
| antibody | | |
| HCDR3 | | |
| Anti-CCR8 | RSSKSLLHSNGNTYLY | 56 |
| antibody | | |
| LCDR1 | | |
| Anti-CCR8 | RMSNLAS | 57 |
| antibody | | |
| LCDR2 | | |
| Anti-CCR8 | MQHLEYPFT | 58 |
| antibody | | |
| LCDR3 | | |
| Anti-CCR8 | EVQLVESGGGLVQPGGSLKLSCAASGFTFNTYAMNWVRQASG | 59 |
| antibody VH | KGLEWVGRIRSKSNNYATYYADSVKDRFTISRDDSKNTAYLQM | |
| | NSLKTEDTAVYYCVRGLLRYRFFDVWGQGTTVTVSS | |
| Anti-CCR8 | DIVMTQSPLSLPVTPGEPASISCRSSKSLLHSNGNTYLYWFLQKP | 60 |
| antibody VL | GQSPQLLIYRMSNLASGVPDRFSGSGSGTDFTLKISRVEAEDVG | |
| | VYYCMQHLEYPFTFGGGTKVEIK | |
| Anti-CCR8 | EVQLVESGGGLVQPGGSLKLSCAASGFTFNTYAMNWVRQASG | 61 |
| antibody heavy | KGLEWVGRIRSKSNNYATYYADSVKDRFTISRDDSKNTAYLQM | |
| chain | NSLKTEDTAVYYCVRGLLRYRFFDVWGQGTTVTVSSASTKGPS | |
| | VFPLAPSSKSTSGGTAALGCLVKDYFPEPVTVSWNSGALTSGV | |
| | HTFPAVLQSSGLYSLSSVVTVPSSSLGTQTYICNVNHKPSNTKV | |
| | DKKVEPKSCDKTHTCPPCPAPELLGGPSVFLFPPKPKDTLMISRT | |
| | PEVTCVVVDVSHEDPEVKFNWYVDGVEVHNAKTKPREEQYN | |
| | STYRVVSVLTVLHQDWLNGKEYKCKVSNKALPAPIEKTISKAK | |
| | GQPREPQVYTLPPSREEMTKNQVSLTCLVKGFYPSDIAVEWESN | |
| | GQPENNYKTTPPVLDSDGSFFLYSKLTVDKSRWQQGNVFSCSV | |
| | MHEALHNHYTQKSLSLSPGK | |
| Anti-CCR8 | DIVMTQSPLSLPVTPGEPASISCRSSKSLLHSNGNTYLYWFLQKP | 62 |
| antibody light | GQSPQLLIYRMSNLASGVPDRFSGSGSGTDFTLKISRVEAEDVG | |
| chain | VYYCMQHLEYPFTFGGGTKVEIKRTVAAPSVFIFPPSDEQLKSG | |
| | TASVVCLLNNFYPREAKVQWKVDNALQSGNSQESVTEQDSKD | |
| | STYSLSSTLTLSKADYEKHKVYACEVTHQGLSSPVTKSFNRGEC | |

### EXAMPLES

In order that this disclosure may be better understood, the following examples are set forth. These examples are for purposes of illustration only and are not to be construed as limiting the scope of the disclosure in any manner.

### Example 1: Procurement of Mouse Anti-Human CCR8 Antibodies

Anti-human CCR8 antibodies (Abcam ab140796 (rabbit polyclonal), BD 566379 (Clone 433H), BioLegend 360602 (Clone L263G8), R&D MAB1429 (Clone 191704), and Sigma HPA042383) were procured from commercial vendors for evaluation by immunohistochemistry (IHC) assays.

### Example 2: Validation of Commercial Anti-Human CCR8 Antibodies

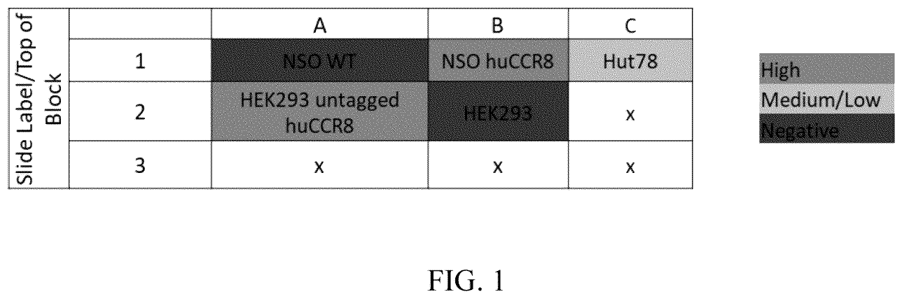

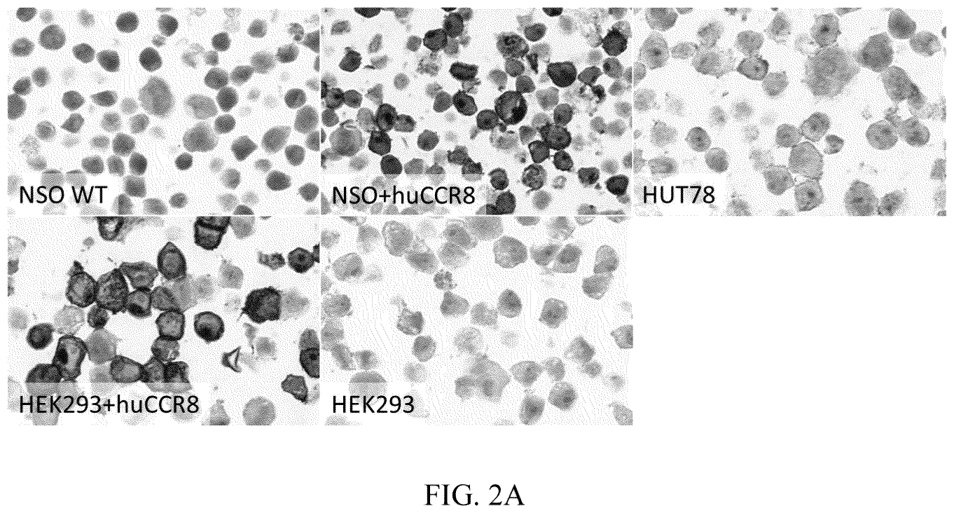

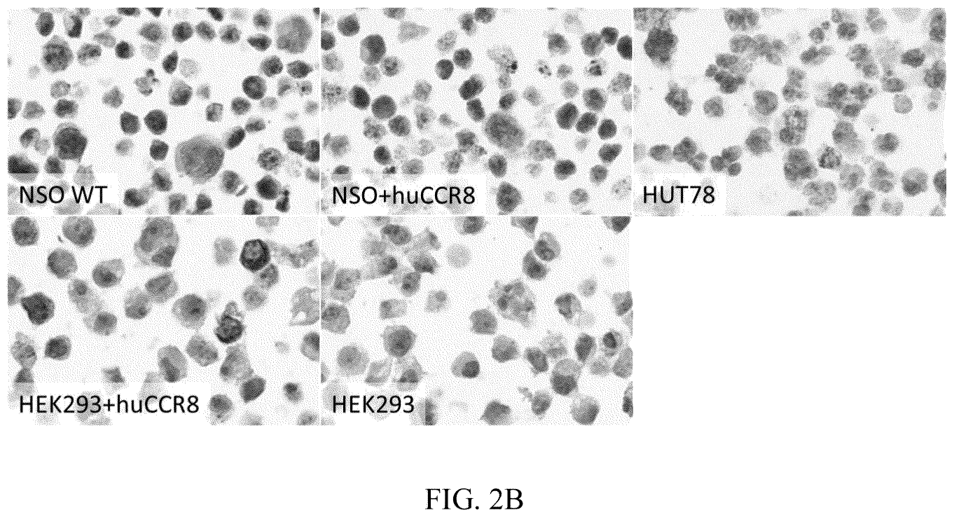

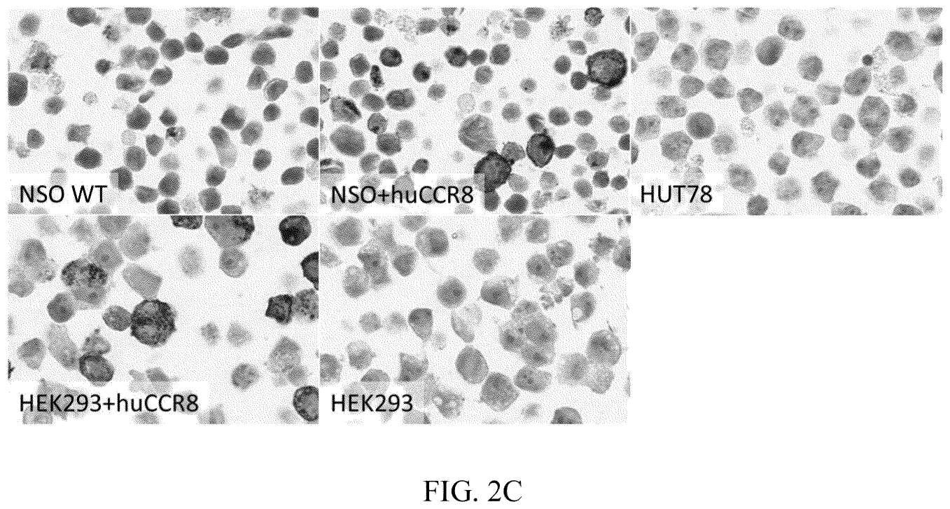

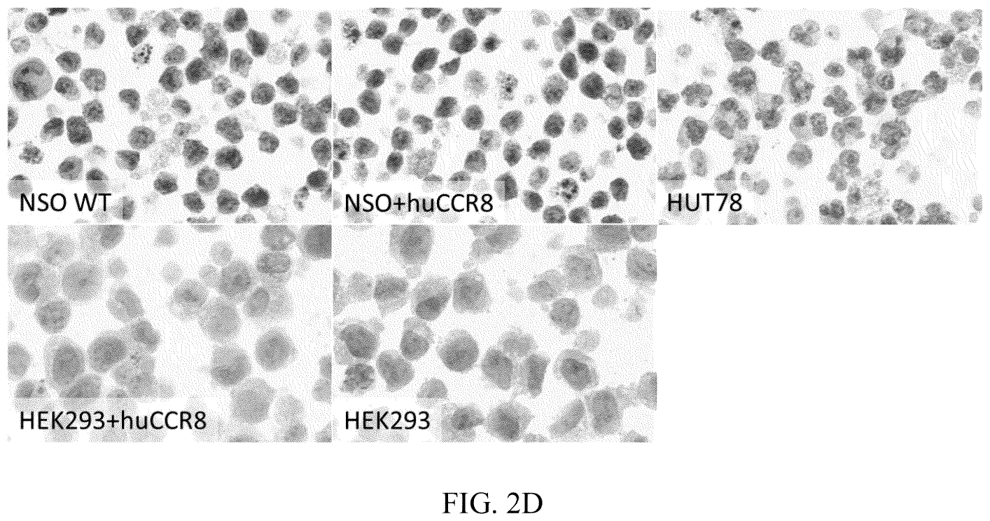

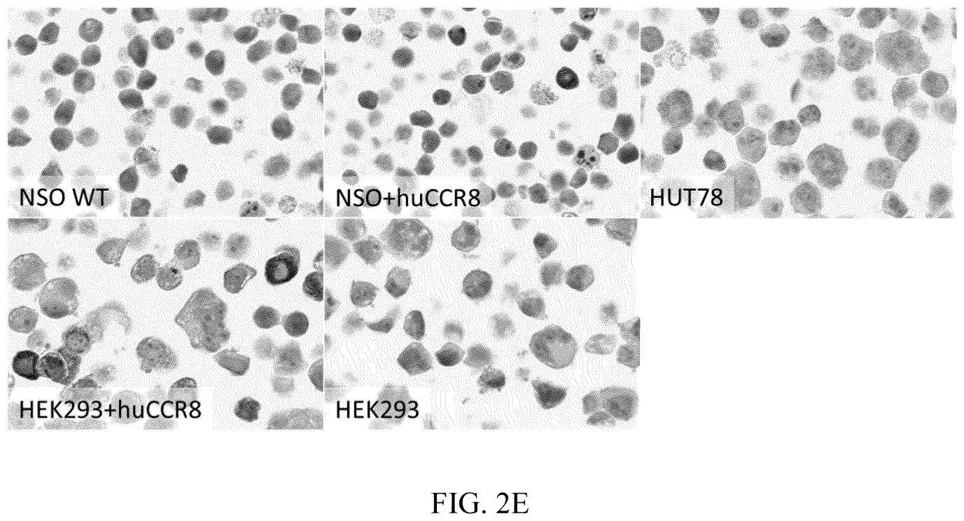

Cell Pellet Array (CPA) validation. The five antibodies, Abcam ab140796 (rabbit polyclonal), BD 566379 (Clone 433H), BioLegend 360602 (Clone L263G8), R&D MAB1429 (Clone 191704), Sigma HPA042383, were initially screened for binding to full-length human CCR8 on a CPA of NSO wildtype (CCR8 negative), NSO huCCR8 (CCR8 high), HEK293 wildtype (CCR8 negative), HEK293 untagged CCR8 (CCR8 high), and Hut78 wildtype (CCR8 medium) cell pellets (TMA-151) (FIG. 1) at various concentrations (0.2, 1.0, and 5 μg/mL). Long heat induced epitope retrieval (HIER) in alkaline buffer was performed by incubation in Borg Decloaker (pH 9.5) and Reveal Decloacker (pH 6.0) solutions from Biocare Medical for 15 minutes at 110 C using Biocare Medical's Decloaking Chamber and IntelliPATH autostainer (Biocare Medical, Pacheco, CA). A total of one—BD 566379 (Clone 433H)—out of the five antibodies showed signal for human CCR8 and was selected for the next phase of testing, while the other four antibodies showed negative, weak, or nonspecific signal for CCR8 staining (FIGS. 2A-2E).

Laboratory Equipment Deletion. At this time in the lab, the Intellipath instrument was retired from the lab and further assessment was performed on the Leica BondRx staining platform (Leica Biosystems).

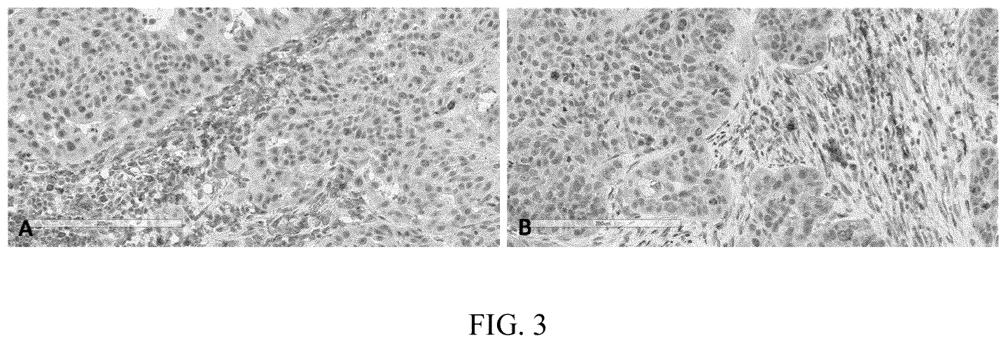

Tissue screen. Next, BD 566379 (Clone 433H) was selected for testing on control H&N SCC tissues (Hu-P-002132 and Hu-P-002140) at 0.25 μg/mL Signal for CCR8 was observed, but non-specific background staining was high (FIG. 3).

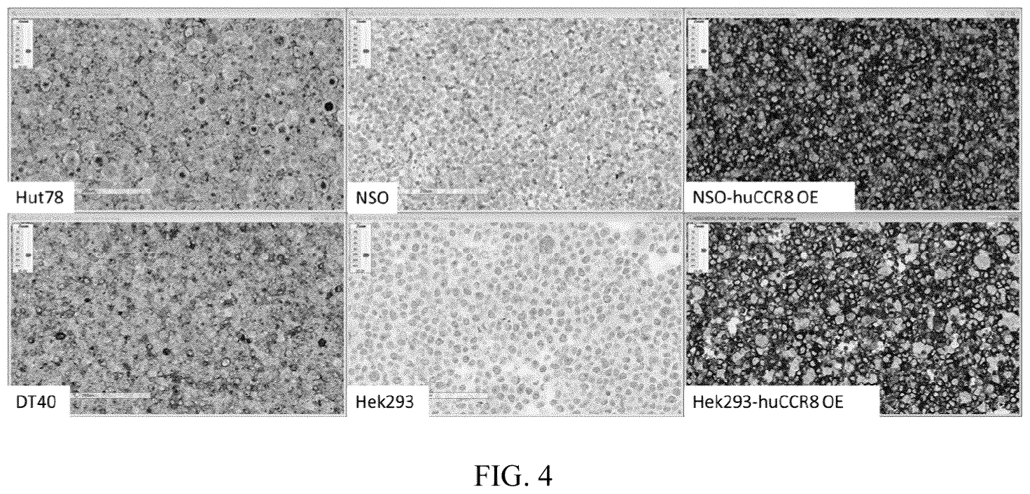

To address background, unconjugated 433H antibody (BD 624084) was procured and validated on CPA TMA-227 with NSO wildtype (CCR8 negative), NSO huCCR8 (CCR8 high), HEK 293 wildtype (CCR8 negative), HEK293 untagged CCR8 (CCR8 high), Hut78 wildtype (CCR8 medium), and DT 40 (CCR8 medium-high) cell pellets (FIG. 4). Long heat induced epitope retrieval (HIER) in alkaline buffer was performed by incubation in ER2 buffer for 20 minutes using the Leica BondRx staining platform (Leica Biosystems) and at concentrations of 0.1, 0.2, 0.5, and 1.0 μg/mL.

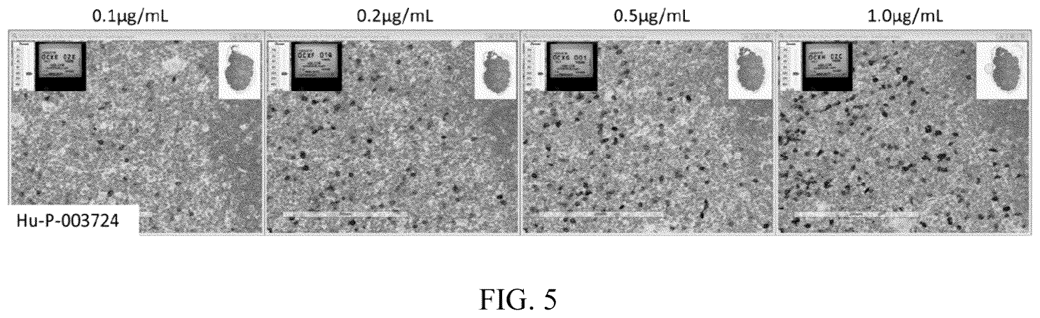

Thymus Tissue validation. BD 624084 was then tested for staining of human thymus samples (Hu-P-003724) using the Bond Rx IHC platform (Leica Biosystems). Staining of the tissue sample was performed by incubating the sample with the antibody at a concentration of 0.1, 0.2, 0.5, and 1.0 μg/mL in Epitope Retrieval 2 (ER2) solution for 20 minutes. Anti-mouse IgG was used as a negative control, and TMA-227 was used as a positive control. The concentration of 0.2 μg/mL was selected as the optimum staining concentration (FIG. 5).

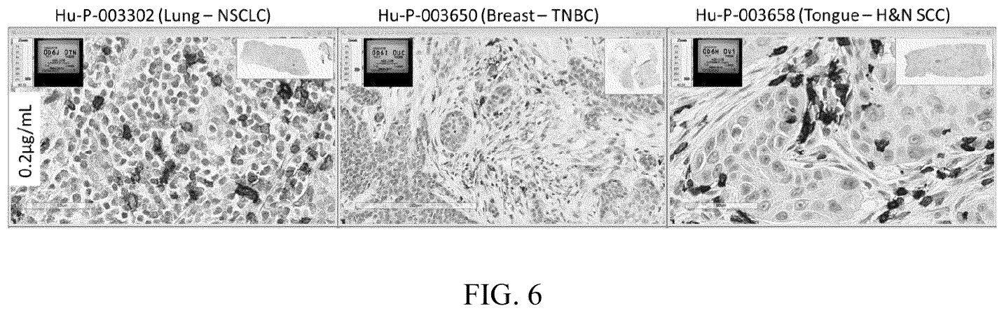

Tumor Tissue validation. To further optimize the conditions for tissue staining with BD 624084, samples from likely tumor indications, HNSCC, Breast carcinoma, and NSCLC-non-squamous, were stained (FIG. 6).

Overall, BD 624084 anti-human CCR8 antibody showed strong and reliable detection of human CCR8 in cells and tissue samples. BD 624084 can be used for detection of CCR8 in human samples, such as human tumor tissue samples. The validation experiments described here support the development of pharmacodynamic (PD) biomarker assays and companion diagnostic (CDx) assays using BD 624084.

### Example 3: Rabbit Anti-Human CCR8 Antibodies do not Compete with CCR8 ECD Domain Targeted Antibody 7-B16

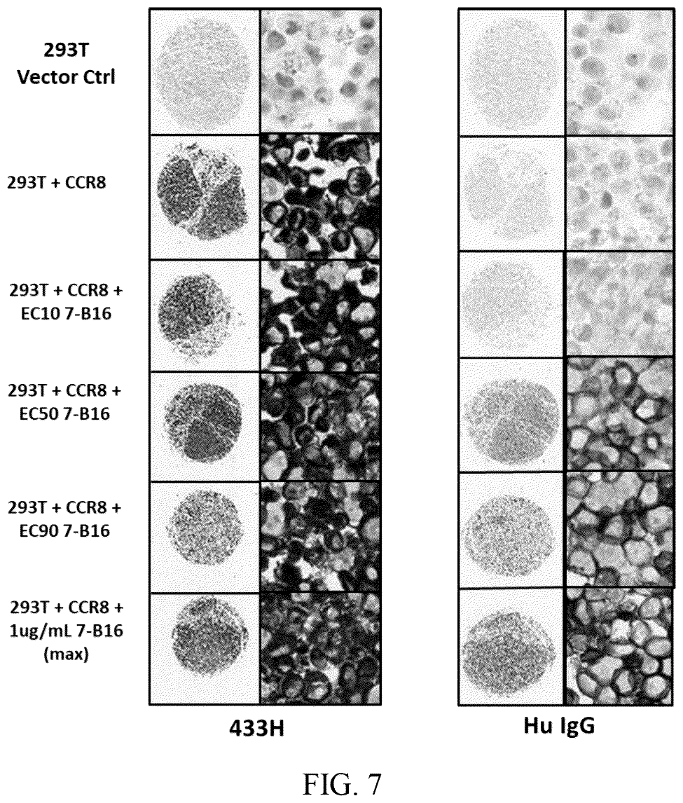

BD 624084 was further validated using a competition assay. Briefly, CCR8-expressing HEK293T cells were stained with each antibody. To test for competition with an exemplary antibody that targets an extracellular N terminal epitope of CCR8 (referred to also as “CCR8 extracellular domain” or “CCR8 ECD”), HEK293T cells were pre-treated with four concentrations (EC10, EC50, EC90 and Emax) of anti-CCR8 antibody 7-B16 (described, e.g., in WO 2021/163064) prior to staining with BD 624084. Cells pre-treated with 7-B16 also showed staining with BD 624084 that was independent of the presence or absence of 7-B16 (FIG. 7), demonstrating that the antibody does not compete for binding with 7-B16. Hu IgG labeling of the treated cell pellets showed that 7-B16 was present on the treated cells. These results illustrate that BD 624084 binds to a different epitope than 7-B16. This example demonstrates that BD 624084 can be used for the development of PD and CDx assays to support therapeutic treatment methods involving anti-CCR8 antibodies that target the CCR8 ECD, including Treg-depleting anti-CCR8 antibodies. The presence of an exemplary CCR8 ECD targeting antibody 7-B16 was not found to interfere with CCR8 staining by BD 624084.

1. A system of specifically binding C-C motif chemokine receptor (CCR8) in a sample obtained from a subject, the system comprising components comprising an isolated antibody or an antigen-binding fragment thereof that binds to human CCR8, wherein the antibody or fragment thereof comprises:
a) a heavy chain variable (VH) region comprising:
i) an HCDR1 comprising the amino acid sequence of SEQ ID NO: 5,
ii) an HCDR2 comprising the amino acid sequence of SEQ ID NO: 6, and
iii) an HCDR3 comprising the amino acid sequence of SEQ ID NO: 7; and
b) a light chain variable (VL) region comprising:
i) an LCDR1 comprising the amino acid sequence of SEQ ID NO: 8,
ii) an LCDR2 comprising the amino acid sequence of SEQ ID NO: 9, and
iii) an LCDR3 comprising the amino acid sequence of SEQ ID NO: 10.

2. A system of detecting C-C motif chemokine receptor (CCR8) expressing cells in a sample obtained from a subject, the system comprising components comprising an isolated antibody or an antigen-binding fragment thereof that binds to human CCR8, wherein the antibody or fragment thereof comprises:
a) a heavy chain variable (VH) region comprising:
i) an HCDR1 comprising the amino acid sequence of SEQ ID NO: 5,
ii) an HCDR2 comprising the amino acid sequence of SEQ ID NO: 6, and
iii) an HCDR3 comprising the amino acid sequence of SEQ ID NO: 7; and
b) a light chain variable (VL) region comprising:
i) an LCDR1 comprising the amino acid sequence of SEQ ID NO: 8,
ii) an LCDR2 comprising the amino acid sequence of SEQ ID NO: 9, and
iii) an LCDR3 comprising the amino acid sequence of SEQ ID NO: 10;
optionally wherein the components further comprise a detection reagent or an amplification reagent.

3. The system of claim 2, wherein the detection of the CCR8 expressing cells in a sample obtained from a subject with cancer indicates the presence of a diseased microenvironment related to CCR8 expression.

4. The system of claim 3, wherein the diseased microenvironment is a tumor microenvironment (TME), and wherein the CCR8 expressing cells comprise tumor infiltrating CCR8+ Treg cells.

5. The system of claim 1, wherein:
a) each component is suspended in a solution, or any combination of components is suspended in the same solution;
b) each component is housed in a container, or any combination of components is housed in a container;
c) each component is provided in a device, or any combination of components is provided in a device; or
d) any combination of (a)-(c).

6. A device for detecting CCR8 expressing cells in a sample obtained from a subject, the device comprising the system of claim 1,
wherein the antibody or fragment thereof is comprised in a reagent that is housed in a container.

7. A kit for detecting CCR8 expressing cells in a sample obtained from a subject, the kit comprising the system of claim 1, and
instructions for using the antibody or fragment thereof for detecting CCR8 expressing cells in the sample obtained from a subject.

8. A method of detecting CCR8 expressing cells in a sample obtained from a subject, comprising:
a) contacting a sample with the system of claim 1;
under conditions sufficient and for a period of time to permit binding between the antibody or fragment thereof and CCR8 expressing cells; and
b) detecting the level of CCR8 expressing cells in said sample.

9. A method of diagnosing a diseased microenvironment associated with CCR8 expression in a subject, comprising:
a) obtaining a sample from the subject;
b) performing the method of claim 8; and
c) associating the level of CCR8 expressing cells with a diseased microenvironment.

10. A method of selecting a subject suspected of having or having cancer for treatment with an anti-CCR8 agent, the method comprising:
a) obtaining a sample from the subject;
b) performing the method of claim 8;
c) comparing the level of the subject's CCR8 expressing cells with a threshold level of CCR8 expressing cells; and
d) selecting the subject for treatment with an anti-CCR8 agent if the level of the subject's CCR8 expressing cells exceed the threshold level of CCR8 expressing cells.

11. The method of claim 10, wherein the CCR8 expressing cell is a CCR8+ Treg cell.

12. A method of treating cancer in a subject in need thereof, the method comprising
a) detecting the level of CCR8 expressing cells in a sample obtained from the subject, detecting comprising:
i) contacting the sample with the system of claim 1;
under conditions sufficient and for a period of time to permit binding between the isolated antibody and/or the binding polypeptide and CCR8;
ii) detecting the level of CCR8 expressing cells in said sample; and
iii) associating the level of CCR8 expressing cells with a diseased microenvironment; and
b) administering to the subject an effective amount of a therapeutic anti-CCR8 agent comprising an antibody or antigen-binding fragment thereof that can specifically bind to CCR8.

13. A method of detecting C-C motif chemokine receptor (CCR8) in a solid tumor sample obtained from a subject, comprising:
a) contacting the solid tumor sample with the system of claim 1;
under conditions sufficient and for a period of time to permit binding between the isolated antibody and/or the antigen-binding fragment thereof and CCR8; and
b) detecting the level of CCR8 expressing cells in said solid tumor sample.

14. A method of selecting a subject having a solid cancer for treatment with a therapeutic anti-CCR8 agent, the method comprising:
a) obtaining a solid tumor sample from the subject;
b) detecting the level of CCR8 expressing cells in the solid tumor sample by performing the method of claim 13;
c) comparing the level of the subject's CCR8 expressing cells with a threshold level of CCR8 expressing cells; and
d) selecting the subject for treatment with an anti-CCR8 agent if the level of the subject's CCR8 expressing cells exceed the threshold level of CCR8 expressing cells.

15. A method of treating a subject having a solid cancer with a therapeutic anti-CCR8 agent, the method comprising:
a) selecting the subject for treatment by performing the method of claim 14; and
b) administering an effective amount of the therapeutic anti-CCR8 agent.

16. A method of monitoring therapeutic efficacy of a treatment with a therapeutic anti-CCR8 agent in a subject having a solid cancer, the method comprising:
a) obtaining a solid tumor sample from the subject;
b) detecting the level of CCR8 expressing cells in the solid tumor sample by performing the method of claim 13; and
c) comparing the detected level of the subject's CCR8 expressing cells with a threshold level of CCR8 expressing cells;
wherein the treatment of the therapeutic anti-CCR8 agent has therapeutic efficacy if the detected level CCR8 expressing cells is depleted relative to the threshold level of CCR8 expressing cells.

17. A method of evaluating the prognosis of a subject having a solid cancer and undergoing treatment with a therapeutic anti-CCR8 agent, the method comprising:
a) obtaining a solid tumor sample from the subject;
(b) detecting the level of CCR8 expressing cells in the solid tumor sample by performing the method of claim 16; and
(c) comparing the detected level of the subject's CCR8 expressing cells with a threshold level of CCR8 or CCR8 expressing cells;
wherein the subject has an improved prognosis if the detected level CCR8 expressing cells is depleted relative to the threshold level of CCR8 expressing cells.

18. The method of claim 16, wherein
i) the threshold level of CCR8 expressing cells is a baseline level of CCR8 expressing cells of the subject prior to receiving the initial treatment with the therapeutic anti-CCR8 agent, or any subsequent treatment with the therapeutic anti-CCR8 agent; or
wherein the CCR8 expressing cells are Treg cells and the detection measures the depletion of CCR8+ Treg cells.

19. (canceled)

20. The method of claim 13, wherein the solid cancer is chosen from squamous cell carcinoma, small cell lung cancer (SCLC), non-small cell lung cancer (NSCLC), squamous NSCLC, non-squamous NSCLC, head and neck cancer, breast cancer, cancer of the esophagus, gastric cancer, gastrointestinal cancer, cancer of the small intestine, liver cancer, hepatocellular carcinoma (HCC), pancreatic cancer (PAC), kidney cancer, renal cell carcinoma (RCC), bladder cancer, cancer of the urethra, cancer of the ureter, colorectal cancer (CRC), colon cancer, colon carcinoma, cancer of the anal region, endometrial cancer, prostate cancer, a fibrosarcoma, neuroblastoma, glioma, glioblastoma, germ cell tumor, pediatric sarcoma, sinonasal natural killer, melanoma, skin cancer, bone cancer, cervical cancer, uterine cancer, carcinoma of the endometrium, carcinoma of the fallopian tubes, ovarian cancer, carcinoma of the cervix, carcinoma of the vagina, carcinoma of the vulva, testicular cancer, cancer of the endocrine system, thyroid cancer, cancer of the parathyroid gland, cancer of the adrenal gland, sarcoma of soft tissue, cancer of the penis, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, solid tumors of childhood, environmentally-induced cancers, virus related cancers, cancers of viral origin, advanced cancer, unresectable cancer, metastatic cancer, refractory cancer, recurrent cancer, and any combination thereof.

21. The method of claim 12, wherein
i) the therapeutic anti-CCR8 agent is selected from TPP-23411, TPP-29338, TPP-27454, TPP-31741, BMS-986340, LM-108, S-531011, FPA157, IPG-7236, ICP-B05, SRF-114, HBM1022, HFB1011, BAY-3375968, IO-1, ZL-1218, GB2101, PSB-114, IPG-A05, PM-1024, DT-7012, BCG-005, GNUV-202, CHS-3318, CTM-033, DT-7012, EGL-002, BGB-A3055, ABBV-514, ABT-863, or CHS-114;
ii) the therapeutic anti-CCR8 agent selected from azirkitug, cafelkibart, lanerkitug, imzokitug, denikitug, or enzelkitug;
iii) the therapeutic anti-CCR8 agent comprises (a) a heavy chain complementary determining region 1 (HCDR1) having the amino acid sequence of SEQ ID NO: 53, (b) an HCDR2 having the amino acid sequence of SEQ ID NO: 54, (c) an HCDR3 having the amino acid sequence of SEQ ID NO: 55, (d) a light chain complementarity determining region 1 (LCDR1) having the amino acid sequence of SEQ ID NO: 56, (e) an LCDR2 having the amino acid sequence of SEQ ID NO: 57, and (f) an LCDR3 having the amino acid sequence of SEQ ID NO: 58;
iv) the therapeutic anti-CCR8 agent comprises (a) a heavy chain variable region (VH) having at least 90%, 91%, 92%, 93%, 94%, 95%, 96%, 97%, 98%, 99%, or 100% sequence identity to the amino acid sequence of SEQ ID NO: 59; and (b) a light chain variable region (VL) having at least 90%, 91%, 92%, 93%, 94%, 95%, 96%, 97%, 98%, 99%, or 100% sequence identity to the amino acid sequence of SEQ ID NO: 60;
v) the therapeutic anti-CCR8 agent comprises (a) a heavy chain variable region (VH) having the amino acid sequence of SEQ ID NO: 59; and (b) a light chain variable region (VL) having the amino acid sequence of SEQ ID NO: 60;
vi) the therapeutic anti-CCR8 agent comprises (a) a heavy chain having at least 90%, 91%, 92%, 93%, 94%, 95%, 96%, 97%, 98%, 99%, or 100% sequence identity to the amino acid sequence of SEQ ID NO: 61; and (b) a light chain having at least 90%, 91%, 92%, 93%, 94%, 95%, 96%, 97%, 98%, 99%, or 100% sequence identity to the amino acid sequence of SEQ ID NO: 62; or
vii) the therapeutic anti-CCR8 agent comprises (a) a heavy chain having the amino acid sequence of SEQ ID NO: 61; and (b) a light chain having the amino acid sequence of SEQ ID NO: 62.

22.-27. (canceled)

28. The method of claim 21, wherein
i) the therapeutic anti-CCR8 agent is afucosylated,
ii) the therapeutic anti-CCR8 agent is denikitug; or
iii) the tissue sample is a formalin-fixed paraffin-embedded (FFPE) tissue sample.

29. (canceled)

30. The system of claim 1, wherein
i) the sample is a tissue sample,
ii) the sample comprises a therapeutic anti-CCR8 antibody; and/or
iii) the antibody or fragment thereof further comprises a detectable label.

31. (canceled)

32. (canceled)

33. The system of claim 30, wherein the therapeutic anti-CCR8 antibody binds to an epitope of
i) the extracellular domain (ECD) of human CCR8, and/or
ii) the amino acid sequence set forth in SEQ ID NO: 16, and wherein the epitope is different from that of the isolated antibody or antigen-binding fragment thereof that binds to human CCR8.

34. (canceled)

35. (canceled)
